(19) United States
(12) Patent Application Publication
(10) Pub. No.: US 2026/0315405 A1
(43) Pub. Date: Oct. 15, 2026

(54) CHITOSAN-BASED NANOCAPSULE SYSTEM FOR USE IN THE PROPHYLACTIC TREATMENT OF AN INFECTION CAUSED BY AN ENVELOPED VIRUS

(71) Applicants: Smart Vitamins SL, Santiago de Compostela (ES); Universidade de Santiago de Compostela, Santiago de Compostela (ES)

(72) Inventors: María José Alonso Fernández, Santiago de Compostela (ES); Alfredo Bermúdez De Castro Franco, Santiago de Compostela (ES); Alicia Rioboo Vidal, Santiago de Compostela (ES); María Dolores Ramona Torres López, Santiago de Compostela (ES)

(73) Assignees: Smart Vitamins SL, Santiago de Compostela (ES); Universidade de Santiago de Compostela, Santiago de Compostela (ES)

(21) Appl. No.: 19/685,711

(22) Filed: May 22, 2026

Related U.S. Application Data
(63) Continuation of application No. PCT/EP2026/060660, filed on Apr. 13, 2026.

(30) Foreign Application Priority Data
Apr. 11, 2025 (EP) 25382382.7
Dec. 3, 2025 (EP) 25383358.6

(51) Int. Cl. A61K 9/51; A61K 9/00; A61K 47/14; A61K 47/24; A61P 31/16

(52) U.S. Cl. CPC A61K 9/5161; A61K 9/0043; A61K 9/5123; A61K 47/14; A61K 47/24; A61P 31/16

(57) ABSTRACT

Chitosan-based nanocapsule system for use in the prophylactic and/or therapeutic treatment of an infection caused by an enveloped virus. The present disclosure refers to the medical field. Particularly, it refers to a chitosan-based nanocapsule system for use in the prophylactic treatment of an infection caused by a respiratory pathogen, preferably an enveloped respiratory virus, wherein the nanocapsule system is administered intranasally.

### TECHNICAL FIELD

The present disclosure refers to the medical field. Particularly, it refers to a chitosan-based nanocapsule system for use in the prophylactic treatment of an infection caused by a respiratory pathogen, preferably an enveloped respiratory virus, wherein the nanocapsule system is administered intranasally.

### BACKGROUND ART

Infectious respiratory diseases are illnesses caused by pathogenic microorganisms—such as viruses, bacteria, or fungi—that invade the respiratory system, leading to infections in structures like the nasal passages, throat, airways, and lungs. They represent a significant global health challenge, with influenza being a major contributor to morbidity and mortality worldwide. The disease burden of these diseases extends beyond respiratory illness, impacting cardiovascular health, exacerbating chronic conditions, and increasing susceptibility to secondary bacterial infections. According to estimates, influenza-associated respiratory deaths range from 290,000 to 650,000 annually, highlighting its substantial impact on public health.

Given the considerable health and economic consequences of infectious respiratory diseases, particularly influenza, there is a pressing need to develop effective prevention strategies.

Vaccination remains the primary and most effective method of influenza prevention, offering significant protection against the virus and its complications. However, the medical community recognizes an ongoing need for improved prophylactic treatments that can enhance existing preventive measures. Such advancements could be particularly beneficial for high-risk individuals, healthcare workers, and in scenarios where additional protection is required.

The development of novel and more effective preventive strategies for infectious respiratory diseases, especially influenza, could significantly reduce the burden on healthcare systems, decrease mortality rates, and improve overall public health outcomes.

In this context, the present disclosure provides a nanocapsule system designed as an intranasal prophylactic treatment against respiratory pathogens, particularly against the influenza virus.

### SUMMARY

As explained in the background art, there is a need for effective preventive strategies for infectious respiratory diseases, particularly for respiratory diseases caused by the influenza virus.

The inventors have developed a chitosan-based nanocapsule that serves as an intranasal prophylactic treatment against respiratory pathogens, particularly the influenza virus. This nanocapsule consists of a chitosan-based shell encapsulating an oily core, which is stabilized through the use of an oil-soluble surfactant (Table 4).

The results provided in this application demonstrate that intranasal administration of these nanocapsules prevent the respiratory pathogen from causing a symptomatic infection, more precisely, from penetrating the nasal epithelium, leading to a complete prevention under the tested conditions of respiratory infection caused by influenza. This is particularly supported by an in vivo experiment performed on mice exposed to the H1N1 PR8 influenza virus. Mice that received intranasal administration of the chitosan-based nanocapsules on days −1, +1, and +3 relative to viral exposure (FIG. 4), thereby providing both prophylactic and post-exposure therapeutic protection, exhibited the following outcomes:
Prevention of weight loss associated with influenza infection: Mice treated with the nanocapsules maintained a body weight comparable to unexposed healthy mice (FIG. 5).
Prevention of inflammatory cytokine release in the lungs and bloodstream: Levels of inflammatory cytokines in the lungs (FIG. 6) of treated mice were comparable to those observed in unexposed mice.
Reduction of inflammatory cytokines in the bloodstream: Levels of inflammatory cytokines in the blood of treated mice were lower than those observed for untreated mice (FIG. 7).
Complete blockage of influenza virus entry into the nasal mucosa: Mice treated with the nanocapsules prior to viral exposure showed lung viral loads comparable to unexposed mice, indicating the nanocapsules effectively prevented viral particles from entering the nasal epithelium (FIG. 8).

This data indicates that intranasal administration of the nanocapsules prevents or reduces influenza infection by forming a protective barrier that blocks viral entry at the primary site of infection (i.e. the nasal mucosa).

In line with these results, the chitosan nanocapsules inhibited the fusion of a SARS-CoV-2 Spike-containing pseudovirus in ACE2 expressing 293T cells as effectively as recombinant murine Fc-ACE2 protein and HIV-1 integration inhibitors like dolutegravir, both of which have shown potential effects against SARS-CoV-2 (FIG. 10). These findings further support the notion that the chitosan nanocapsules form a protective barrier that inhibits viral entry into cells and suggest that the protective effect observed against the influenza virus is also applicable to other viral respiratory infections, such as SARS-CoV-2.

Unlike previously disclosed chitosan-based nanocapsules, these nanocapsules do not act as adjuvants, modulators of active ingredient activity, or delivery vehicles for other active compounds. Notably, these nanocapsules are not loaded with additional active ingredients. Instead, they possess an inherent prophylactic effect resulting from the combination of the nanocapsules' components. This effect was demonstrated by prevention of infection in animal models treated intranasally on days −1, +1, and +3 relative to viral exposure (FIG. 8).

Additionally, in vitro toxicity studies performed in 293-ACE2 cells (FIG. 2) confirmed that the chitosan-based nanocapsule formulation is non-toxic, providing significantly higher cell viability compared to alternative formulations. Even at high concentrations (dilution 1:4), the nanocapsules maintained cell viability above 50%, supporting their safety for nasal administration.

In conclusion, these nanocapsules offer a threefold advantage: they act as a protective barrier against viral entry, exhibit an intrinsic prophylactic effect, and ensure low toxicity—making them a promising innovation for preventing influenza and other infectious respiratory diseases.

Remarkably, the outstanding efficacy of these nanocapsules is achieved with extremely low amounts of chitosan, which brings several advantages. Using such minimal concentrations ensures exceptionally low toxicity while maintaining a high level of prophylactic effectiveness, as demonstrated by the prevention or reduction of influenza infection in treated mice. Additionally, reducing the quantity of chitosan required for the treatment enhances patient comfort, as lower doses typically result in a more tolerable administration experience. This aspect is important for improving patient adherence to the prophylactic regimen, ultimately increasing the overall success of the preventive strategy.

Additional in vitro results further demonstrated that the prophylactic and antiviral effects of the chitosan-based nanocapsules extend beyond influenza virus and apply to other enveloped viruses, both respiratory and non-respiratory. These experiments confirmed that the chitosan-based nanocapsules can inhibit viral entry and replication in several cellular models (Examples 8.1-8.4):
Absence of cytotoxicity: The nanocapsules showed no detectable cytotoxicity across the concentrations tested in HEK293T-ACE2 cells (FIG. 11), Vero E6 cells (FIGS. 15, 16 and 19), and U87 CD4+ CXCR4+ cells (FIG. 17), confirming their safety profile in vitro in human and animal cell lines. These findings further support the low toxicity previously demonstrated in vivo (FIG. 2), reinforcing that the nanocapsules are well tolerated under conditions relevant to prophylactic use.
Inhibition of SARS-CoV-2 pseudoviral fusion (Example 8.1): The nanocapsules completely inhibited SARS-CoV-2 pseudoviral entry into ACE2-expressing cells at the highest non-cytotoxic concentrations, with a clear dose-dependent effect (FIGS. 11 and 12). The magnitude of inhibition was comparable to that observed with the positive controls—soluble ACE2-mFc and dolutegravir—highlighting that the nanocapsules can achieve antiviral efficacy equivalent to established inhibitors of viral entry. In contrast, the non-formulated components of the nanocapsules, tested at concentrations equivalent to those present in the complete formulation, did not display any significant antiviral effect (FIGS. 13 and 14). This indicates that the antiviral activity is not attributable to the isolated components, but rather to their supramolecular organization within the nanocapsule.
Inhibition of replication of SARS-CoV-2 variants (D614G, JN1, KP2) (Example 8.2): Nanocapsules inhibited replication of multiple SARS-CoV-2 variants—specifically the D614G, JN1, and KP2 strains—in Vero E6 cells in a dose-dependent manner, at concentrations that did not cause detectable cytotoxicity (FIGS. 15A and 16B). The antiviral activity observed was comparable to that of the reference antiviral remdesivir, which represents the current standard of care for SARS-CoV-2 inhibition. This finding is particularly relevant as it demonstrates that the nanocapsules can reach antiviral efficacy equivalent to that of a clinically validated antiviral drug. Conversely, the non-formulated components tested as unassembled mixtures at equivalent concentrations failed to inhibit viral replication (FIGS. 15B and 16B), confirming that the structural organization of the nanocapsule is essential for the observed effect.
Inhibition of respiratory syncytial virus (RSV) replication (Example 8.4): Nanocapsules inhibited RSV replication in Vero E6 cells in a concentration-dependent manner at non-cytotoxic concentrations (FIGS. 19A and 20). The non-formulated components did not reproduce this antiviral effect (FIG. 19B) at the same concentrations, further supporting that the assembled nanocapsule, rather than its isolated constituents, confers the observed antiviral protection.
Inhibition of HIV-1 replication (Example 8.3): The nanocapsules inhibited HIV-1 replication at non-cytotoxic concentrations in U87 CD4+ CXCR4+ cells, a validated in vitro model for HIV infection (FIGS. 17 and 18). The antiviral activity observed was comparable to that of dolutegravir, a clinically approved antiretroviral drug, underscoring the robustness of the nanocapsules' antiviral action (FIG. 18). Non-formulated components tested at identical concentrations exhibited no inhibitory activity (FIG. 18), reinforcing that the antiviral effect arises exclusively from the nanocapsule structure and not from its individual ingredients.

Together, these results demonstrate that the nanocapsules of the present disclosure are effective against a wide range of infectious viruses, including influenza virus, SARS-CoV-2, RSV, and HIV-1. Although these viruses differ substantially in their genetic material, replication strategies, and tropism, many of which are enveloped viruses, meaning that each virus is surrounded by a lipid bilayer encasing its capsid, which is essential for viral fusion and entry into host cells. The consistent antiviral effect observed across all these viruses suggests that the mechanism of action of the nanocapsules is non-specific and relies on their ability to interact with or disrupt the viral lipid envelope, preventing the fusion and entry of viral particles into host cells. In this regard, the experiments conducted in Vero E6 cells—which genetically lack the ability to produce type I interferons (IFN-I), key mediators of the innate antiviral immune response—are particularly informative. The observation that the nanocapsules still exert a clear antiviral effect in these cells provides strong evidence that their activity is independent of an IFN-I-mediated innate immune response and instead arises from a direct, physical impediment to viral fusion, supporting the conclusion that the nanocapsules act through a non-specific, virus-disrupting mechanism. The experiments conducted using SARS-CoV-2 pseudovirus in ACE2-expressing cells (Example 8.1) are also particularly informative in this regard. ACE2-expressing cells are engineered to present the viral receptor ACE2 on their surface, allowing SARS-CoV-2 entry, but they are not immune cells and therefore lack the genetic machinery to produce immunoglobulins; in particular, they cannot generate neutralizing antibodies. The observation that the nanocapsules completely inhibited SARS-CoV-2 pseudoviral entry into these cells suggests that their antiviral effect does not rely on stimulation of the immune system and further supports that the activity of the nanocapsules arises from a direct, physical interference with viral fusion rather than from immune-mediated mechanisms.

This conclusion is further reinforced by the analysis of the non-formulated components. Taken together, these results clearly demonstrate that the individual components of the nanocapsules—although they could in principle elicit an innate immune response-do not exhibit a measurable antiviral or prophylactic effect on their own when used at concentrations comparable to those present in the complete nanocapsule formulation (FIGS. 13, 14, 16, 19, and 18). This strongly supports that the antiviral activity observed is an emergent property of the nanostructured assembly, whose combined components generate a physical barrier that prevents viral fusion.

These findings also suggest that the nanocapsules could be used as a broad-spectrum prophylactic platform:
For respiratory enveloped viruses (influenza, SARS-CoV-2, RSV), intranasal administration provides direct protection at the site of viral entry.
For other enveloped viruses such as HIV-1, local application at the potential site of infection (e.g., vaginal or rectal) may provide therapeutic and/or prophylactic antiviral activity.

Overall, the new data confirm that the chitosan-based nanocapsules are not limited to influenza or specific viral receptors but act as a universal physical barrier effective against multiple enveloped viruses. This broad antiviral spectrum, combined with their low toxicity and ease of administration, positions these nanocapsules as a novel and safe prophylactic tool for the prevention of infectious diseases caused by enveloped viruses.

In addition, we have collected further data using a MucilAir human nasal epithelium model and a murine biodistribution study, both of which further support the efficacy, safety, and localized mode of action of the chitosan-based nanocapsules of the present disclosure (also referred to as SV RESPVIR B-I formulation) as a nasal prophylactic intervention against viral infections.

In a physiologically relevant model of the human nasal mucosa, SV RESPVIR B-I nanoparticles demonstrated excellent tolerability, with no detectable cytotoxic effects at any of the tested concentrations (9.35-74.8 μg/μl). Cell viability in the MucilAir model remained comparable to untreated controls, confirming that the formulation is well tolerated under conditions relevant to nasal application (FIG. 21).

Importantly, SV RESPVIR B-I did not activate the type I interferon (IFN) innate immune response, as evidenced by the absence of meaningful induction of IFNα1 and IFNβ1 gene expression following nanoparticle treatment. In contrast, robust IFN pathway activation was observed only with the canonical positive control Poly I:C. These findings indicate that the antiviral activity of the formulation is not mediated by immune stimulation, supporting a non-immune, physically driven mechanism of action (FIG. 22).

Consistent with this interpretation, SV RESPVIR B-I nanoparticles exhibited potent antiviral activity against SARS-CoV-2 in the MucilAir model. Treatment resulted in a dose-dependent reduction of viral infectivity of up to 91%, whereas the corresponding non-formulated components showed significantly lower antiviral efficacy at equivalent concentrations. This confirms that the antiviral effect is an emergent property of the assembled nanoparticle structure, rather than of its individual components (FIG. 23).

The formulation also demonstrated a prolonged residence time and sustained antiviral efficacy on the nasal epithelium. Full antiviral activity was maintained for up to 8 hours post-application, and approximately 70% of antiviral efficacy was retained after 24 hours. These results indicate that SV RESPVIR B-I remains associated with the epithelial surface for extended periods while preserving functional activity, a key attribute for effective nasal prophylaxis (FIG. 24).

In vivo biodistribution studies further corroborated these findings. Following intranasal administration of rhodamine-labelled SV RESPVIR B-I in mice, fluorescence imaging revealed that the formulation was predominantly retained within the nasal cavity, with minimal signal detected in distal organs. Ex vivo analysis confirmed that the highest fluorescence levels were localized in the nasal epithelium, while liver, kidneys, lungs, and plasma showed negligible signals, indicating absence of systemic distribution (FIGS. 25 and 26).

Together, these results demonstrate that the chitosan-based nanocapsules of the present disclosure acts as a locally retained, non-toxic nasal formulation with sustained antiviral activity and a mechanism of action that does not rely on immune activation. The strong confinement of the formulation to the nasal cavity, combined with its prolonged residence time and robust antiviral efficacy, supports a local mechanical barrier mode of action, reinforcing its suitability as a safe and effective prophylactic strategy against viral exposure at the primary site of infection.

The disclosure provides:
A nanocapsule system, which comprises or consists of:
   a) a surface layer comprising chitosan
   b) an oily core, and
   c) a surfactant,
for use in the prophylactic and/or therapeutic treatment of an infection caused by a respiratory pathogen, preferably a respiratory virus, in a subject in need thereof, wherein the nanocapsule is administered to the subject intranasally, or alternatively, for use in the prophylactic and/or therapeutic treatment of an infection caused by a respiratory pathogen in a subject in need thereof, wherein the nanocapsule system is administered intranasally to the subject

In a preferred embodiment, optionally in combination with any of the previous or subsequent embodiments, the prophylactic treatment comprises or consists of preventing the respiratory pathogen from causing a symptomatic infection or the prophylactic treatment comprises preventing the respiratory pathogen from causing a symptomatic infection.

In another preferred embodiment, optionally in combination with any of the previous or subsequent embodiments, the prophylactic treatment comprises or consists of preventing the respiratory pathogen from causing a symptomatic infection by preventing the respiratory pathogen from entering the nasal epithelium by forming a protective barrier, or by preventing, or at least reducing, entry of the respiratory pathogen into the nasal epithelium via formation of a protective barrier.

In another preferred embodiment, optionally in combination with any of the previous or subsequent embodiments, the nanocapsule system is for use in the prophylactic and/or therapeutic treatment of an infection caused by a respiratory virus. Preferably, the respiratory virus is selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses, bocavirus, and enteroviruses. More preferably, the respiratory virus is influenza virus. Preferably, the influenza virus is an influenza A virus, preferably H1N1 influenza.

In another preferred embodiment, optionally in combination with any of the previous or subsequent embodiments, the nanocapsule system is for the specific use in the prophylactic and/or therapeutic treatment of an infection caused by a respiratory virus is selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses, bocavirus, and enteroviruses. More preferably, the respiratory virus is influenza virus.

In another preferred embodiment, optionally in combination with any of the previous or subsequent embodiments, the nanocapsule system is for use in the prophylactic and/or therapeutic treatment of an infection caused by the influenza virus. Preferably, the influenza virus is an influenza A virus, preferably H1N1 influenza.

In another preferred embodiment, optionally in combination with any of the previous or subsequent embodiments, the nanocapsule system is for use in the prophylactic and/or therapeutic treatment of an infection caused by an influenza A virus, preferably H1N1 influenza.

In another preferred embodiment, optionally in combination with any of the subsequent embodiments, the oily core comprises or consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof.

In another preferred embodiment, optionally in combination with any of the previous or subsequent embodiments, the oily core comprises or consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, and the prophylactic treatment comprises or consists of preventing the respiratory pathogen from causing a symptomatic infection.

In another preferred embodiment, optionally in combination with any of the previous or subsequent embodiments, the oily core comprises or consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, the nanocapsule system is for the specific use in the prophylactic treatment of an infection caused by the influenza virus, wherein the prophylactic treatment comprises or consists of preventing the respiratory pathogen from causing a symptomatic infection. Preferably, the influenza virus is an influenza A virus, preferably H1N1 influenza.

In another preferred embodiment, optionally in combination with any of the previous or subsequent embodiments, the oil consists of 90% w/w of triglycerides with three fatty acids having an aliphatic tail of 6 to 12 carbon atoms.

In another preferred embodiment, optionally in combination with any of the previous or subsequent embodiments, the oil consists of at least of 90% w/w of a mixture of caprylic (C8) and capric (C10) triglycerides.

In another preferred embodiment, optionally in combination with any of the previous or subsequent embodiments, the oil consists of at least 90% w/w of a mixture of caprylic (C8) and capric (C10) triglycerides in a w/w ratio between 80:20 and 50:50, more preferably between 75:25 and 65:35, even more preferably 60:40. Even more preferably, the oil consists of at least 90% w/w of Miglyol® 812N. Even more preferably, the oil is Miglyol® 812N.

In another preferred embodiment, optionally in combination with any of the previous or subsequent embodiments, the oil consists of at least 90% w/w of Miglyol® 812N. Even more preferably, the oil is Miglyol® 812N.

In another preferred embodiment, optionally in combination with any of the previous or subsequent embodiments, the oily core consists of at least 90% w/w of Miglyol® 812N.

In another preferred embodiment, optionally in combination with any of the previous or subsequent embodiments, the surfactant is an oil-soluble surfactant suitable for stabilizing the oily core. Preferably, the oil-soluble surfactant is a phospholipid-based compound selected from the list consisting of lecithin, phosphatidylglycerol, phosphatidylserine, phosphatidylinositol, diphosphatidylglycerol, phosphatidic acid, phosphatidylcholine and phosphatidylethanolamine. Preferably, the phospholipid-based compound is selected from the list consisting of Lipoid S75 (soy phospholipid with 70% phosphatidylcholine), hydrogenated lecithin, phosphatidylcholine, LIPOID S 45 (lecithin fraction with phosphatidylcholine content≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥90%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%). More preferably, the oil-soluble surfactant is Lipoid S75 (soy phospholipid with 70% phosphatidylcholine).

In another preferred embodiment, optionally in combination with any of the previous or subsequent embodiments, the oil-soluble surfactant is a phospholipid-based compound selected from the list consisting of lecithin, phosphatidylglycerol, phosphatidylserine, phosphatidylinositol, diphosphatidylglycerol, phosphatidic acid, phosphatidylcholine and phosphatidylethanolamine.

In another preferred embodiment, optionally in combination with any of the previous or subsequent embodiments, the phospholipid-based compound is selected from the list consisting of Lipoid S75 (soy phospholipid with 70% phosphatidylcholine), hydrogenated lecithin, phosphatidylcholine, LIPOID S 45 (lecithin fraction with phosphatidylcholinecontent≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥90%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%).

In another preferred embodiment, optionally in combination with any of the previous or subsequent embodiments, the phospholipid-based compound is Lipoid S75 (soy phospholipid with 70% phosphatidylcholine).

In another preferred embodiment, optionally in combination with any of the previous or subsequent embodiments, the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, the oil consists of at least 90% w/w of a mixture of caprylic (C8) and capric (C10) triglycerides in a w/w ratio between 80:20 and 50:50, the surfactant is Lipoid S75 (soy phospholipid with 70% phosphatidylcholine), and the nanocapsule system is for the specific use in the prophylactic treatment of an infection caused by the influenza virus.

The disclosure further provides:
A nanocapsule system, which comprises:
   a) a surface layer comprising chitosan;
   b) an oily core; and
   c) a surfactant;
for use in the prophylactic and/or therapeutic treatment of an infection caused by an enveloped respiratory virus in a subject in need thereof, wherein the nanocapsule system is administered to the subject intranasally; or alternatively, for use in the prophylactic and/or therapeutic treatment of an infection caused by a respiratory virus, preferably by an enveloped respiratory virus, in a subject in need thereof, wherein the nanocapsule system is administered intranasally to the subject.

In a preferred embodiment, optionally in combination with any of the previous or subsequent embodiments, the surface layer does not comprise an antigen, or alternatively, the surface layer does not comprise an antigen capable of eliciting an immune response against the respiratory virus

In a preferred embodiment, optionally in combination with any of the previous or subsequent embodiments, the surface layer does not comprise an antigen capable of eliciting an immune response against the enveloped respiratory virus; or alternatively, the surface layer does not comprise an antigen capable of eliciting an antigen-specific immune response against the enveloped respiratory virus

In another preferred embodiment, optionally in combination with any of the previous or subsequent embodiments, the antigen is selected from the list consisting of a viral protein of the enveloped respiratory virus or a fragment, variant, analogue, or synthetic mimic thereof, wherein the viral protein is selected from a surface glycoprotein, a fusion protein, a receptor-binding domain, a membrane-associated protein, or a nucleoprotein.

In another preferred embodiment, optionally in combination with any of the previous or subsequent embodiments, the surface layer consists of chitosan and, optionally, one or more components, with the proviso that the one or more components do not comprise an antigen; or alternatively, the surface layer consists of chitosan and, optionally, one or more non-antigenic components.

In another preferred embodiment, optionally in combination with any of the previous or subsequent embodiments, the surface layer consists of chitosan and, optionally, one or more components, with the proviso that the one or more components do not comprise an antigen capable of eliciting an immune response against the enveloped respiratory virus.

In another preferred embodiment, optionally in combination with any of the previous or subsequent embodiments, the surface layer consists of chitosan and, optionally, one or more components, wherein the one or more components are selected from the group consisting of, or alternatively, wherein the one or more pharmaceutically acceptable non-antigenic components are selected from the group consisting of:
i. a polysaccharide, selected from the group consisting of cellulose, hydroxypropyl methylcellulose, methylcellulose, ethylcellulose, carbomer, dextran, pullulan, hyaluronic acid, alginate, gellan gum, xanthan gum, inulin, pectin, and mixtures thereof;
ii. a polyol or sugar alcohol, selected from the group consisting of glycerol, sorbitol, mannitol, xylitol, erythritol, propylene glycol, and mixtures thereof;
iii. a synthetic polymer, selected from the group consisting of polyethylene glycol (PEG), polypropylene glycol (PPG), polyvinyl alcohol (PVA), polyvinylpyrrolidone (PVP), poloxamers, poly(2-ethyl-2-oxazoline), and mixtures thereof;
iv. a peptide or amino-acid-based compound lacking antigenic epitopes, selected from the group consisting of short neutral peptides (520 aa), glycine, alanine, serine, valine, leucine, isoleucine, proline, hydroxyproline, and mixtures thereof;
v. a lipid or fatty-acid-derived compound, selected from the group consisting of phosphatidylcholine, phosphatidylethanolamine, sphingomyelin, ceramides, mono-, di- and triglycerides, fatty acids (C8-C22), fatty alcohols (C8-C22), and mixtures thereof;
vi. a zwitterionic or non-ionic surfactant-compatible stabilizer, selected from the group consisting of lecithin, polysorbates, sorbitan esters, sucrose esters, polyethylene glycol esters, polyglyceryl esters, and mixtures thereof;
vii. an inorganic or mineral component, selected from the group consisting of silica, colloidal silica, calcium phosphate, magnesium phosphate, titanium dioxide, and mixtures thereof;
viii. a plasticizer or flexibility-modifying agent, selected from the group consisting of triethyl citrate, citrate esters, sebacates, adipates, and mixtures thereof;
ix. a buffer or ionic excipient, selected from the group consisting of sodium chloride, potassium chloride, phosphate salts, citrate salts, acetate salts, and mixtures thereof;
x. and mixtures thereof.

In another preferred embodiment, optionally in combination with any of the previous or subsequent embodiments, the surface layer consists of chitosan.

In another preferred embodiment, optionally in combination with any of the previous or subsequent embodiments, the oily core consists of an oil.

In another preferred embodiment, optionally in combination with any of the previous or subsequent embodiments, the oil is selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof.

In another preferred embodiment, optionally in combination with any of the previous or subsequent embodiments, the oil is selected from the list consisting of mineral oil, squalene oil, silicone oil, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C15 alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, fractionated coconut oil, castor oil, long-chain monoglycerides, long-chain diglycerides, long-chain triglycerides, medium-chain monoglycerides, medium-chain diglycerides, medium-chain triglycerides, oleic acid, linoleic acid, oleyl alcohol, isostearyl alcohol, ethyl oleate, synthetic or semi-synthetic derivatives and combinations thereof, wherein the oil preferably consists of medium-chain triglycerides.

In another preferred embodiment, optionally in combination with any of the previous or subsequent embodiments, the oil consists of medium-chain triglycerides having a mixture of caprylic (C8) and capric (C10) triglycerides, wherein the mixture of caprylic (C8) and capric (C10) triglycerides is preferably in a w/w ratio between 80:20 and 50:50, more preferably between 75:25 and 65:35, even more preferably 60:40.

In another preferred embodiment, optionally in combination with any of the previous or subsequent embodiments, the enveloped respiratory virus is selected from the group consisting of influenza virus, coronavirus, respiratory syncytial virus (RSV), parainfluenza virus, metapneumovirus, henipavirus, cytomegalovirus (CMV), varicella-zoster virus (VZV), herpes simplex virus (HSV), measles virus, mumps virus, rubella virus, and any combination thereof.

In another preferred embodiment, optionally in combination with any of the previous or subsequent embodiments, the enveloped respiratory virus is the influenza virus.

In another preferred embodiment, optionally in combination with any of the previous or subsequent embodiments, the enveloped respiratory virus is the respiratory syncytial virus (RSV).

In another preferred embodiment, optionally in combination with any of the previous or subsequent embodiments, the enveloped respiratory virus is the severe acute respiratory syndrome coronavirus 2 (SARS-CoV-2).

In another preferred embodiment, optionally in combination with any of the previous or subsequent embodiments, the surfactant is an oil-soluble surfactant, preferably selected from the list consisting of lecithin, phosphatidylglycerol, phosphatidylserine, phosphatidylinositol, diphosphatidylglycerol, phosphatidic acid, phosphatidylcholine and phosphatidylethanolamine.

In another preferred embodiment, optionally in combination with any of the previous or subsequent embodiments, the phospholipid-based compound is selected from the list consisting of Lipoid S75 (soy phospholipid with 70% phosphatidylcholine), hydrogenated lecithin, phosphatidylcholine, LIPOID S 45 (lecithin fraction with phosphatidylcholine content≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥0%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%).

In another preferred embodiment, optionally in combination with any of the previous or subsequent embodiments, the phospholipid-based compound is Lipoid S75 (soy phospholipid with 70% phosphatidylcholine).

### DESCRIPTION OF THE FIGURES

FIGS. 1A-1B 1. Images of the (FIG. 1A) preparation of the aqueous phase and (FIG. 1B) preparation of the nanocapsule emulsion explained in Example 1.

FIG. 2. Cell viability curves for the 3 formulations studied. In each group of bars, from left to right: PArg NCs, Prot NCs, CS NCs.

FIG. 3. IL-6 levels in the supernatants of RAW264.7 cells inflamed with LPS prior to administration of the treatment indicated in each case. The levels of significance between the groups are indicated. The correspondence between asterisks and level of significance is: *=α<0.1, **=α<0.01.

FIG. 4. Experimental design and treatment of efficacy experiments in mice.

FIG. 5. Graph of weight loss over time in mice receiving different types of treatments.

FIG. 6. Levels of inflammatory cytokines in the lungs of animals receiving different treatments. In each group of bars, from left to right: PBS, PBS+influenza, CS NCs+influenza.

FIG. 7. Levels of inflammatory cytokines in the blood of infected animals treated with PBS (control) or our formulation. In each group of bars, from left to right: PBS+influenza, CS NCs+influenza.

FIG. 8. Viral load levels in the lungs of animals that received different treatments.

FIG. 9. Cellular cytotoxicity assessment by cell metabolic activity, performed in 293-ACE2 cells 3 times in triplicates for 8 dilutions of our nanocapsules (NPs).

FIG. 10. RLUs from HIV-1-Luc pseudotyped with SARS-CoV-2 spike in 293-ACE2 cells, where a dose-response effect of the NPs decreasing viral fusion is observed.

FIG. 11. Cytotoxic effect in HEK293T-ACE2 cells exposed to decreasing concentrations of SV RESPVIR B-I nanoparticles (NPs). The nanoparticles were tested at serial 1:2 dilutions ranging from 100 ul to 0.78 ul.

FIG. 12. Antiviral effect measured by inhibition of viral fusion in HEK293T-ACE2 cells exposed to decreasing concentrations of SV RESPVIR B-I nanoparticles (NPs). NPs were tested at serial 1:2 dilutions ranging from 100 ul to 12.5 ul. Statistical differences were assayed with a Mann Whitney t test.

FIG. 13. Cytotoxic effect in HEK 293T-ACE2 cells exposed to decreasing concentrations of SV RESPVIR B-I nanoparticles (NPs) and equivalent non-formulated components. Non-formulated components were tested at increasing concentrations exceeding 1 to 4-fold the concentration of nanoparticles.

FIG. 14. Antiviral effect measured by inhibition of viral fusion in HEK293T-ACE2 cells exposed to decreasing concentrations of non-formulated components. These reagents were tested at serial 1:2 dilutions comparing non-formulated components exceeding 1 to 4-fold the concentration of nanoparticles.

FIGS. 15A-15B. Antiviral activity measured by reduction of cytopathic effect after treatment with SV RESPVIR B-I formulation in Vero E6 cells infected with a low MOI of SARS-CoV-2. Nanoparticles (NP) (FIG. 15A) and non-formulated components (FIG. 15B) were tested in concentrations ranging from 4.675-34.40 μg/μl on Vero E6 cells infected with a low MOI of SARS-CoV-2 D614G and Omicron JN1. Data shows normalized (%) luciferase Relative Light Units (RLUs). IC50 for each virus is indicated. Lines, top to bottom: D614G, Omicron JN.1, Drug-associated cytotoxic effect.

FIGS. 16A-16B. Antiviral activity measured by reduction of cytopathic effect after treatment with SV RESPVIR B-I formulation in Vero E6 cells infected with a high MOI of SARS-CoV-2. Lines, top to bottom: Omicron KP.2, Drug-associated cytotoxic effect.

FIG. 17. Cytotoxic effect in U87 CD4+ CXCR4+ cells exposed to decreasing concentrations of SV RESPVIR B-I nanoparticles (NPs). The nanoparticles were tested at serial 1:2 dilutions ranging from 100 ul to 6.25 ul.

FIG. 18. Antiviral effect measured by reduction of cytopathic effect after treatment with SV RESPVIR B-I formulation (NPs) in U87 CD4+ CXCR4+ cells infected with HIV-1. NPs and non-formulated components were tested at serial 1:2 dilutions ranging from 25 ul to 6.25 ul. Data were normalized to non-infected cells transformed to 100% viable. Statistical differences were assayed with a Mann Whitney t test.

FIGS. 19A-19B. Antiviral effect measured by reduction of cytopathic effect after treatment with SV RESPVIR B-I formulation in Vero E6 cells infected with RSV. Nanoparticles (FIG. 19A) and the mix of components used to formulate nanoparticles (FIG. 19B) were tested in concentrations ranging from 4.7-37.4 μg/μl on Vero E6 cells infected with RSV. Cells were assayed 6 dpi to detect luciferase by Relative Light Units (RLUs) using cell titer glo. Data were normalized to non-infected cells transformed to 100% viable and fitted to a non-linear variable slope curve. Lines, top to bottom: RSV-GFP, Drug-associated cytotoxic effect.

FIG. 20. Antiviral activity of 50 uM of Remdesivir in Vero E6 cells infected with RSV. Remdesivir (50 uM) was tested on Vero E6 cells infected with RSV. Cells were assayed 6 dpi to detect luciferase by Relative Light Units (RLUs) using cell titer glo. Data were normalized to non-infected cells transformed to 100% viable and fitted to a non-linear variable slope curve.

FIG. 21. Cytotoxicity assessment of SV RESPVIR B-I nanoparticles in the MucilAir human nasal epithelium model. MucilAir tissues were treated with SV RESPVIR B-I nanoparticles (NPs) or the corresponding non-formulated components at concentrations ranging from 9.35 to 74.8 μg/μl for 72 hours. Cell viability was measured using the CellTiter-Glo® luminescence assay and expressed as relative light units (RLU). Results are normalized to untreated cells (No Drug, set to 100%). Data represent mean±SD of experimental duplicates (n=2).

FIG. 22. Type I interferon gene expression analysis following treatment with SV RESPVIR B-I nanoparticles in the MucilAir model. MucilAir tissues were treated for 72 hours with SV RESPVIR B-I nanoparticles or corresponding components at concentrations from 9.35 to 74.8 μg/μl. Gene expression levels of IFNα1 (FIG. 22A) and IFNβ1 (FIG. 22B) were quantified by RT-PCR and normalized to untreated controls (No Drug). Poly I:C (12.5 ng/μl) was used as a positive control for interferon pathway activation. Data represent mean±SD of 1-3 independent experiments with technical duplicates.

FIG. 23. Antiviral activity of SV RESPVIR B-I nanoparticles against SARS-CoV-2 in the MucilAir human nasal epithelium model. MucilAir tissues were treated with SV RESPVIR B-I nanoparticles or corresponding components at concentrations of 18.70 μg/μl and 74.8 μg/μl and subsequently infected with nanoluciferase-expressing SARS-CoV-2 (nLuc-SCV2). Viral infectivity was quantified after 72 hours using the Nano-Glo® luminescence assay. Results are normalized to infected untreated controls (Virus, set to 100%). Data represent mean±SD of three independent experiments (n=3) with technical duplicates.

FIG. 24. Residence time and sustained antiviral efficacy of SV RESPVIR B-I nanoparticles in the MucilAir human nasal epithelium model. SV RESPVIR B-I nanoparticles (74.8 μg/μl) were applied to the apical surface of MucilAir tissues, which were subsequently infected with SARS-CoV-2 (nLuc-SCV2) at 0, 4, 8, 24, or 48 hours post-application. Antiviral activity was assessed 72 hours after infection by measuring nanoluciferase activity. Results demonstrate sustained antiviral efficacy over time, expressed relative to infected untreated controls.

FIG. 25. In vivo and ex vivo fluorescence imaging of mice following intranasal administration of rhodamine-labelled SV RESPVIR B-I. Mice received intranasal administration of rhodamine-labelled SV RESPVIR B-I formulation (20 μl per nostril). Representative whole-body in vivo fluorescence imaging was performed at 24 hours post-administration. Corresponding ex vivo fluorescence imaging of harvested tissues shows predominant localization of the formulation in the nasal region, with minimal signal detected in distal organs.

FIG. 26. Quantitative biodistribution of rhodamine-labelled SV RESPVIR B-I following intranasal administration in mice. Fluorescence intensity (relative fluorescence units, RFU) was quantified after rhodamine extraction from ex vivo tissue samples collected 24 hours after intranasal administration of SV RESPVIR B-I. Tissues analyzed included nasal epithelium, lungs, liver, kidneys, and plasma. RFU values are shown relative to the administered dose (TO), demonstrating predominant retention in the nasal epithelium and negligible systemic distribution.

### DETAILED DESCRIPTION OF THE EMBODIMENTS

### General Considerations

Each embodiment disclosed herein is contemplated as being applicable to each of the other disclosed embodiments. Thus, all combinations of the various elements described herein are within the scope of the disclosure.

Unless otherwise stated, the terms used in the present application have the meanings indicated herein. Terms that are not defined herein should be given the meanings that would be given to them by a person skilled in the art in the context of this disclosure.

For the purposes of the present disclosure, any ranges given include both the lower and the upper endpoints of the range.

In the context of the present disclosure the following general terms are defined:
The term “comprising” means including, but it is not limited to, whatever follows the word “comprising”. Thus, use of the term “comprising” indicates that the listed elements are required or mandatory, but that other elements are optional and may or may not be present. The term “comprising” also includes the term “consists of”.
The term “consisting of” means including, and it is limited to, whatever follows the phrase “consisting of”. Thus, the phrase “consisting of” indicates that the listed elements are required or mandatory, and that no other elements may be present.

In view of the above-mentioned results, a first aspect refers to a nanocapsule system (hereinafter “nanocapsule system of the disclosure”), which comprises a surface layer comprising chitosan, for use in the prophylactic treatment of an infection caused by a respiratory pathogen in a subject in need thereof, wherein the nanocapsule is administered to the subject intranasally.

Thus, the first aspect of the disclosure refers to a nanocapsule system.

The term “nanocapsule system” refers to either an individual nanocapsule or a population of nanocapsules, each having a structure of less than 1 μm in diameter composed of a core surrounded by a polymeric shell or membrane, forming a well-defined reservoir.

It is noted that as used herein, the expression “an enveloped respiratory virus” refers to any virus belonging to the class of enveloped respiratory viruses, whereas the expression “the enveloped respiratory virus” refers to the specific enveloped respiratory virus against which a given antigen is directed

Hereinafter, “first excluded antigen” refers to an antigen capable of eliciting an immune response against the enveloped respiratory virus. In a preferred embodiment, the first excluded antigen is an antigen capable of eliciting an adaptive immune response against the enveloped respiratory virus. In a more preferred embodiment, the embodiment further specifies that an excluded antigen is considered capable of eliciting an adaptive immune response against the enveloped respiratory virus if, when administered in vivo in an model, it induces a measurable virus-specific adaptive immune response, evidenced by (i) the production of virus-specific antibodies at least 2-fold higher than baseline levels in the same subject or in a matched control group, or (ii) the activation or expansion of virus-specific T or B cells at least 2-fold higher than baseline levels in the same subject or in a matched control group, as determined by enzyme-linked immunosorbent assay (ELISA) for antibody quantification, virus-specific T-cell or B-cell frequency measurements by flow cytometry using fluorophore-labelled tetramers or activation markers or interferon-γ or cytokine-release assays such as ELISPOT or intracellular cytokine staining (ICS). In an even more preferred embodiment, the embodiment further specifies that the excluded antigen is selected from the list consisting of a viral protein of an enveloped respiratory virus or a fragment, variant, analogue, or synthetic mimic thereof. In an even more preferred embodiment, the embodiment further specifies that the viral protein is selected from a surface glycoprotein, a fusion protein, a receptor-binding domain, a membrane-associated protein, or a nucleoprotein. In an even more preferred embodiment, the embodiment further specifies that the viral protein of an enveloped respiratory virus or a fragment, variant, analogue, or synthetic mimic thereof, is a viral protein of the enveloped respiratory virus or a fragment, variant, analogue, or synthetic mimic thereof.

Hereinafter, “second excluded antigen” refers to an antigen from an enveloped respiratory virus wherein the antigen is capable of eliciting an adaptive immune response against the enveloped respiratory virus. Preferably, the second excluded antigen is an antigen selected from the list consisting of a viral protein of an enveloped respiratory virus or a fragment, variant, analogue, or synthetic mimic thereof. Preferably, the viral protein is selected from a surface glycoprotein, a fusion protein, a receptor-binding domain, a membrane-associated protein, or a nucleoprotein.

Hereinafter, “third excluded antigen” refers to an antigen from the enveloped respiratory virus wherein the antigen is capable of eliciting an adaptive immune response against the enveloped respiratory virus. Preferably, the third excluded antigen is selected from the list consisting of a viral protein of the enveloped respiratory virus or a fragment, variant, analogue, or synthetic mimic thereof. Preferably, the viral protein is selected from a surface glycoprotein, a fusion protein, a receptor-binding domain, a membrane-associated protein, or a nucleoprotein.

Hereinafter, “fourth excluded antigen” refers to an antigen. The term “antigen” refers to a molecule or molecular structure that contains at least one epitope capable of being specifically recognized by components of the adaptive immune system—namely, an antibody, a B-cell receptor, or a T-cell receptor—when presented by a major histocompatibility complex (MHC) molecule in a manner that allows the receptor to recognize the epitope within a non-sensitized immune system. The term “epitope” refers to a discrete molecular feature of a molecule or molecular structure that is specifically recognized by an antibody, a B-cell receptor, or a T-cell receptor when presented by a major histocompatibility complex (MHC) molecule in a manner that permits recognition by the receptor. The term “non-sensitized immune system” refers to the immune system of an organism that has not previously been exposed to or primed against the specific molecule or molecular structure being tested, such that any observed recognition or binding occurs in the absence of prior immunological memory specific to that molecule. In the context of this definition, an antigen or an epitope is considered specifically recognized by components of the adaptive immune system—namely, an antibody, a B-cell receptor, or a T-cell receptor—when the interaction occurs preferentially with the relevant antibody or receptor compared to unrelated antibodies or receptors, such that the epitope or antigen is uniquely identified by the immune system within a non-sensitized immune system. As will be well understood by a person skilled in the art, such specific recognition can be assessed and demonstrated using a variety of immunological techniques, including but not limited to: enzyme-linked immunosorbent assays (ELISA), surface plasmon resonance (SPR), biolayer interferometry (BLI), flow cytometry using fluorophore-labeled tetramers or activation markers, intracellular cytokine staining (ICS), enzyme-linked immunospot (ELISPOT) assays measuring cytokine release, or other binding, functional, or activation assays that allow differentiation between specific and non-specific interactions. The skilled person will appreciate that the particular technique or combination of techniques can be chosen based on the nature of the epitope, the antibody or receptor, the sample type, and the experimental context, provided that the assay reliably demonstrates preferential interaction with the relevant antibody or receptor. Preferably, an excluded antigen is defined as a molecule or molecular structure that contains at least one epitope capable of being specifically recognized by an antibody, a B-cell receptor, or a T-cell receptor in a non-sensitized immune system, and wherein such recognition is demonstrated by measurable antigen-specific immune recognition, evidenced by (i) binding to or induction of antigen-specific antibodies at levels at least 2-fold higher than baseline levels in the same subject or in a matched control sample, as determined by enzyme-linked immunosorbent assay (ELISA), surface plasmon resonance (SPR), or other quantitative antibody-binding assays, or (ii) activation, proliferation, or expansion of antigen-specific B or T cells at levels at least 2-fold higher than baseline levels in the same subject or in a matched control sample, as determined by flow cytometry using fluorophore-labeled tetramers, activation markers, intracellular cytokine staining (ICS), or enzyme-linked immunospot (ELISPOT) assays measuring cytokine release.

With regard to the embodiments relative to the first, second or third excluded antigen:
Preferably, the immune response is an adaptive immune response.
Preferably, an excluded antigen is considered capable of eliciting an adaptive immune response against the enveloped respiratory virus meaning the enveloped respiratory virus for which a virus-specific adaptive immune response is detected, if, when administered in vivo in an model, it induces a measurable virus-specific adaptive immune response, evidenced by (i) the production of virus-specific antibodies at least 2-fold higher than baseline levels in the same subject or in a matched control group, or (ii) the activation or expansion of virus-specific T or B cells at least 2-fold higher than baseline levels in the same subject or in a matched control group, as determined by enzyme-linked immunosorbent assay (ELISA) for antibody quantification, virus-specific T-cell or B-cell frequency measurements by flow cytometry using fluorophore-labelled tetramers or activation markers or interferon-γ or cytokine-release assays such as ELISPOT or intracellular cytokine staining (ICS).
Preferably, the excluded antigen is selected from the list consisting of a viral protein of an enveloped respiratory virus or a fragment, variant, analogue, or synthetic mimic thereof.
Preferably, the excluded antigen capable of eliciting an immune response against the enveloped respiratory virus is selected from the list consisting of a viral protein of an enveloped respiratory virus or a fragment, variant, analogue, or synthetic mimic thereof.
Preferably, the viral protein is selected from a surface glycoprotein, a fusion protein, a receptor-binding domain, a membrane-associated protein, or a nucleoprotein.

In a preferred embodiment, the nanocapsule system of the disclosure comprises: a) a surface layer comprising chitosan, b) an oily core, and c) a surfactant.

In another preferred embodiment, the nanocapsule system does not comprise the first, second, third and/or fourth excluded antigen. Preferably, the nanocapsule system does not comprise any first, second, third or fourth excluded antigen.

In another preferred embodiment, the nanocapsule system comprises or consist of a) a surface layer comprising chitosan, b) an oily core, and c) a surfactant, and optionally, one or more components, with the proviso that the one or more components do not comprise the first, second, third and/or fourth excluded antigen(s).

In a more preferred embodiment, the nanocapsule system comprises or consist of a) a surface layer comprising chitosan, b) an oily core, and c) a surfactant.

Preferably, the nanocapsule system is a spherical droplet where the oily core is encapsulated within the chitosan shell, with the surfactant forming a stabilizing boundary at the interface. The nanocapsule system of the disclosure may be obtained by combining these elements in various ratios, as would be recognized by a person skilled in the art. Specifically, the ratios may range from 0.5% to 20% w/w for the oily core, from 0.05% to 20% w/w for the surfactant, and from 0.01% to 10% w/w for the chitosan surface layer. For instance, the nanocapsule system can be implemented using the specific quantities of each component exemplified in Table 3.

The term “spherical droplet” refers to a roughly spherical, three-dimensional structure formed by a liquid or semi-liquid core, maintaining a nearly uniform radius due to surface tension forces, resulting in a smooth, curved outer surface.

The term “encapsulated” refers to the state in which the core material—whether liquid, solid, or gas—is fully enclosed or surrounded by an outer layer or shell, providing protection, controlled release, or functional separation from the surrounding environment.

The term “stabilizing boundary” refers to a functional layer, typically formed by surfactants or emulsifiers, that sits at the interface between two immiscible phases (e.g., oil and water) to reduce surface tension, prevent coalescence, and ensure the structural integrity and longevity of the droplet.

The term “surface layer” refers to the outermost layer of a nanocapsule system, which acts as a protective barrier that surrounds the core material. This layer helps to stabilize the nanocapsule and protect it from environmental factors. In a preferred embodiment, the surface layer comprises or consists of at least 70% w/w of chitosan, preferably at least 80% w/w, more preferably at least 90% w/w. Preferably, the surface layer consists of chitosan.

In another preferred embodiment, the surface layer does not comprise the first, second, third and/or fourth excluded antigen.

In another preferred embodiment, the surface layer consists of chitosan and, optionally, one or more components, with the proviso that the one or more components do not comprise the first, second, third and/or fourth excluded antigen(s). Preferably, the one or more components do not comprise the first, second, third and/or fourth excluded antigen(s) are selected from the group consisting of: (i) a polysaccharide, selected from the group consisting of cellulose, hydroxypropyl methylcellulose, methylcellulose, ethylcellulose, carbomer, dextran, pullulan, hyaluronic acid, alginate, gellan gum, xanthan gum, inulin, pectin, and mixtures thereof; (ii) a polyol or sugar alcohol, selected from the group consisting of glycerol, sorbitol, mannitol, xylitol, erythritol, propylene glycol, and mixtures thereof; (iii) a synthetic polymer, selected from the group consisting of polyethylene glycol (PEG), polypropylene glycol (PPG), polyvinyl alcohol (PVA), polyvinylpyrrolidone (PVP), poloxamers, poly(2-ethyl-2-oxazoline), and mixtures thereof; (iii) a peptide or amino-acid-based compound lacking antigenic epitopes, selected from the group consisting of short neutral peptides (:20 aa), glycine, alanine, serine, valine, leucine, isoleucine, proline, hydroxyproline, and mixtures thereof; (iv) a lipid or fatty-acid-derived compound, selected from the group consisting of phosphatidylcholine, phosphatidylethanolamine, sphingomyelin, ceramides, mono-, di- and triglycerides, fatty acids (C8-C22), fatty alcohols (C8-C22), and mixtures thereof; (v) a zwitterionic or non-ionic surfactant-compatible stabilizer, selected from the group consisting of lecithin, polysorbates, sorbitan esters, sucrose esters, polyethylene glycol esters, polyglyceryl esters, and mixtures thereof; (vi) an inorganic or mineral component, selected from the group consisting of silica, colloidal silica, calcium phosphate, magnesium phosphate, titanium dioxide, and mixtures thereof; (vii) a plasticizer or flexibility-modifying agent, selected from the group consisting of triethyl citrate, citrate esters, sebacates, adipates, and mixtures thereof; (viii) a buffer or ionic excipient, selected from the group consisting of sodium chloride, potassium chloride, phosphate salts, citrate salts, acetate salts, and mixtures thereof; (ix) and mixtures thereof. None of these components have antigens capable of eliciting an immune response against the enveloped respiratory virus.

In another preferred embodiment, the surface layer consists of chitosan and, optionally, one or more components selected from the group consisting of: (i) a polysaccharide, selected from the group consisting of cellulose, hydroxypropyl methylcellulose, methylcellulose, ethylcellulose, carbomer, dextran, pullulan, hyaluronic acid, alginate, gellan gum, xanthan gum, inulin, pectin, and mixtures thereof; (ii) a polyol or sugar alcohol, selected from the group consisting of glycerol, sorbitol, mannitol, xylitol, erythritol, propylene glycol, and mixtures thereof; (iii) a synthetic polymer, selected from the group consisting of polyethylene glycol (PEG), polypropylene glycol (PPG), polyvinyl alcohol (PVA), polyvinylpyrrolidone (PVP), poloxamers, poly(2-ethyl-2-oxazoline), and mixtures thereof; (iii) a peptide or amino-acid-based compound lacking antigenic epitopes, selected from the group consisting of short neutral peptides (≤20 aa), glycine, alanine, serine, valine, leucine, isoleucine, proline, hydroxyproline, and mixtures thereof; (iv) a lipid or fatty-acid-derived compound, selected from the group consisting of phosphatidylcholine, phosphatidylethanolamine, sphingomyelin, ceramides, mono-, di- and triglycerides, fatty acids (C8-C22), fatty alcohols (C8-C22), and mixtures thereof; (v) a zwitterionic or non-ionic surfactant-compatible stabilizer, selected from the group consisting of lecithin, polysorbates, sorbitan esters, sucrose esters, polyethylene glycol esters, polyglyceryl esters, and mixtures thereof; (vi) an inorganic or mineral component, selected from the group consisting of silica, colloidal silica, calcium phosphate, magnesium phosphate, titanium dioxide, and mixtures thereof; (vii) a plasticizer or flexibility-modifying agent, selected from the group consisting of triethyl citrate, citrate esters, sebacates, adipates, and mixtures thereof; (viii) a buffer or ionic excipient, selected from the group consisting of sodium chloride, potassium chloride, phosphate salts, citrate salts, acetate salts, and mixtures thereof; (ix) and mixtures thereof. None of these components have antigens capable of eliciting an immune response against the enveloped respiratory virus.

In more preferred embodiment, the surface layer consists of chitosan.

The term “chitosan” refers to a biocompatible, biodegradable, and non-toxic polysaccharide derived from chitin, composed primarily of β-(1→4)-linked D-glucosamine units, with varying amounts of N-acetyl-D-glucosamine residues. Its properties are typically characterized by parameters such as degree of deacetylation (DDA) and molecular weight (MW). The degree of deacetylation refers to the extent to which the acetyl groups (—COCH3) have been removed from the chitosan molecule. Chitosan is derived from chitin, and the deacetylation process involves removing these acetyl groups, transforming chitin into chitosan. As an expert in the field would recognize, a common method to measure the degree of deacetylation is by using infrared spectroscopy (FTIR). The intensity of the absorption band related to the acetyl group (—C═O stretching at around 1650 cm⁻¹) can be compared to other bands (like the amine group at 1590 cm⁻¹) to determine the amount of deacetylation. Another method involves titration techniques, where the amino group concentration in the chitosan sample is measured and compared to the theoretical maximum amount that would be present if the chitosan were fully deacetylated. On the other hand, the average molecular weight of chitosan refers to the mass of a single molecule of chitosan, averaged over the entire sample. As an expert in the field would recognize, a commonly used technique to determine the molecular weight of chitosan is gel permeation chromatography (GPC), also known as size-exclusion chromatography (SEC). GPC separates molecules based on their size, and by comparing the retention time of the sample with a set of standards of known molecular weight, it is possible to determine the average molecular weight of the chitosan sample. Another method is light scattering or viscometry, where the viscosity of a chitosan solution is measured and related to the molecular weight using specific models.

Importantly, unless otherwise specified in these embodiments, the term ‘chitosan’ also includes pharmaceutically acceptable salts and functional derivatives of chitosan.

The term “pharmaceutically acceptable salt” refers to a salt form of chitosan that is formed by reacting chitosan with a pharmaceutically acceptable acid, resulting in a compound that is suitable for use in pharmaceutical applications. Such salts must be safe for human or animal use and are typically soluble in water or other suitable solvents, providing enhanced bioavailability or stability without causing adverse effects. These salts are particularly useful in the preparation of chitosan-based nanoparticles or nanocapsules, as they improve the solubility, dispersibility, and stability of chitosan in aqueous environments, which are important for effective encapsulation, drug delivery, and controlled release. The ability of these salts to solvate chitosan enhances the formation of nanoparticles or nanocapsules, allowing for better encapsulation of active pharmaceutical ingredients (APIs) or bioactive agents. Additionally, the enhanced solubility and stability of these salts in aqueous solutions allow for efficient production of nanocapsules, as well as improved bioavailability and release profiles of the encapsulated substances. Examples of pharmaceutically acceptable salts of chitosan include, but are not limited, to chitosan hydrochloride (chitosan HCl), chitosan lactate, chitosan acetate, chitosan glutamate and chitosan sulfate. These salts facilitate the use of chitosan in various formulations, improving its solubility, stability, and bioavailability in pharmaceutical or therapeutic applications, especially in the preparation of nanocapsules for controlled release and targeted delivery of drugs or bioactive molecules.

The term “functional derivate” refers to a derivative of chitosan in which the chemical structure of the chitosan polymer is modified to introduce specific functional groups or properties. These modifications are intended to enhance the chitosan's characteristics, such as solubility, biocompatibility, charge, or cellular uptake, without altering the fundamental polymer backbone. Such derivatives are particularly useful in the preparation of chitosan-based nanocapsules, as they provide improved properties such as solubility, charge density, and bioactivity, which are essential for the effective encapsulation, stability, and controlled release of therapeutic agents. For example, functional derivatives like quaternized chitosan (N,N,N-trimethyl chitosan chloride—TMC) improve the permeability of nanocapsules and their solubility in aqueous environments, making them ideal for drug delivery applications. Carboxymethyl chitosan (CMC) enhances solubility and introduces a negative charge at physiological pH, which is beneficial for the preparation of stable nanoparticles with targeted delivery capabilities. Hydroxypropyl chitosan improves the bioactivity and water solubility, often used in ophthalmic formulations where high bioavailability and sustained release are important. Additionally, chitosan oligosaccharides, with their shorter polymer chains, exhibit enhanced bioactivity and are used in therapeutic applications for improved cellular interaction.

These functional derivatives allow chitosan to be tailored for specific applications, particularly in nanoparticle or nanocapsule formulations, providing benefits such as improved solubility, targeted drug delivery, enhanced interaction with biological systems, and optimized release profiles of encapsulated substances. Examples of functional derivatives of chitosan include, but are not limited to quaternized chitosan (N,N,N-trimethyl chitosan chloride—TMC), carboxymethyl chitosan (CMC), hydroxypropyl chitosan and chitosan oligosaccharides.

The examples set in the following section were performed using chitosan HCl (Heppe Medical Chitosan GmbH, Product No. 54040 and 55040). The properties of the specific chitosan batches used in the examples are summarized in Table 1.

TABLE 1
Properties of the specific chitosan batches used in the examples.

| Parameter | Limit | Result |
|---|---|---|
| Degree of deacetylation | 75%-90% | 82-83% |
| Molecular weight | To be measured | 109 kDa |
| pH | 4.0-6.0 | 4.1-4.5 |
| Chlorides | 10-20% | 14.9% |
| Protein content | ≤0.3% | 0.1-0.2% |

In a preferred embodiment, the chitosan has a degree of deacetylation comprised from 75% to 90% w/w, preferably from 80% to 85% w/w, more preferably 83% w/w.

In another preferred embodiment, the chitosan has an average molecular weight comprised from 90 kDa to 120 kDa, preferably from 100 kDa to 120 kDa, more preferably from 104 kDa to 114 kDa, even more preferably 109 kDa.

In another preferred embodiment, the chitosan has a pH comprised from 4.0 to 6.0, preferably from 4.0 to 5.0, more preferably from 4.1 to 4.5.

In another preferred embodiment, the chitosan is in its native chemical structure. The term “native chemical structure” refers to the unaltered polymeric backbone and natural functional groups of chitosan, free from covalent chemical modifications such as sulfation, sulfonation, or other substitutions that change the primary molecular structure. This term includes chitosan in its free base form, protonated form, or pharmaceutically acceptable salts resulting from ionic interactions—such as chitosan hydrochloride—provided these forms retain the original backbone and functional integrity of the chitosan molecule. However, this term does not encompass functional derivatives of chitosan, such as quaternized chitosan, carboxymethyl chitosan, or other modified forms that introduce additional functional groups or alter the chitosan's chemical structure.

Preferably, the chitosan is a chitosan salt. More preferably, the chitosan is a chitosan salt selected from the list consisting of chitosan hydrochloride (chitosan HCl), chitosan lactate, chitosan acetate, chitosan glutamate and chitosan sulfate. Even more preferably the chitosan is chitosan hydrochloride. Even more preferably, the chitosan hydrochloride a percentage of chloride comprised from 10% to 20% w/w, preferably from 13% to 17% w/w, more preferably from 14% to 16% w/w, even more preferably 14.9%.

In another preferred embodiment, the chitosan has a protein content is equal or lower than 0.3% w/w, preferably 0.2% w/w.

Other embodiments related to the properties of chitosan set forth below. Moreover, all the embodiments of the first aspect of the disclosure apply herein:
In a particular preferred embodiment the chitosan has a degree of deacetylation comprised from 75% to 90% w/w, preferably from 80% to 85% w/w, more preferably 83% w/w, and an average molecular weight comprised from 90 kDa to 120 kDa, preferably from 100 kDa to 120 kDa, more preferably from 104 kDa to 114 kDa, even more preferably 109 kDa.
In a particular preferred embodiment the chitosan has a degree of deacetylation comprised from 80% to 85% w/w, more preferably 83% w/w, and an average molecular weight comprised from 100 kDa to 120 kDa, more preferably from 104 kDa to 114 kDa, even more preferably 109 kDa.
In a particular preferred embodiment the chitosan has a degree of deacetylation comprised from 80% to 85% w/w, more preferably 83% w/w, and an average molecular weight comprised from 104 kDa to 114 kDa, even more preferably 109 kDa.
In a particular preferred embodiment the chitosan has a degree of deacetylation comprised from 75% to 90% w/w, preferably from 80% to 85% w/w, more preferably 83% w/w, an average molecular weight comprised from 90 kDa to 120 kDa, preferably from 100 kDa to 120 kDa, more preferably from 104 kDa to 114 kDa, even more preferably 109 kDa, and is in its native chemical structure.
In a particular preferred embodiment the chitosan has a degree of deacetylation comprised from 80% to 85% w/w, more preferably 83% w/w, an average molecular weight comprised from 100 kDa to 120 kDa, more preferably from 104 kDa to 114 kDa, even more preferably 109 kDa, and is in its native chemical structure.
In a particular preferred embodiment the chitosan has a degree of deacetylation comprised from 80% to 85% w/w, more preferably 83% w/w, an average molecular weight comprised from 104 kDa to 114 kDa, even more preferably 109 kDa, and is in its native chemical structure.

The term “oily core” refers to the hydrophobic, oil-based interior of a nanocapsule or similar encapsulated structure, designed to solubilize, protect, and deliver lipophilic active ingredients. This core is typically composed of one or more oils, which can include natural, synthetic, or semi-synthetic lipids, such as triglycerides, fatty acid esters, essential oils, hydrocarbons, or silicone oils. The oily core serves as a reservoir that helps encapsulate poorly water-soluble compounds, enhancing their stability, bioavailability, or controlled release. The specific composition of the oily core can be tailored to the desired physical and chemical properties of the formulation, including viscosity, polarity, and compatibility with other components like surfactants or polymers that form the outer shell of the nanocapsule.

The examples set in the following section were performed using an oily core of Miglyol® 812N. Miglyol® 812N is a medium-chain triglyceride oil composed of a mixture of caprylic acid (C8:0) 50-80% and capric acid (C10:0) 20-50%, typically derived from coconut or palm kernel oil. The standarized ratio for Miglyol® 812N is a 60:40 w/w ratio of caprylic acid (C8:0) to capric acid (C10:0).

In a preferred embodiment (hereinafter embodiment #O1), the oily core comprises or consists of an oil. Preferably, the oily core comprises or consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, any synthetic or semi-synthetic derivatives and combinations thereof. More preferably, the oily core comprises or consists of medium-chain triglycerides (i.e. triglycerides with three fatty acids having an aliphatic tail of 6 to 12 carbon atoms). Even more preferably, the oily core comprises or consists of medium-chain triglycerides having a mixture of caprylic (C8) and capric (C10) triglycerides. Preferably, the mixture of caprylic (C8) and capric (C10) triglycerides is in a w/w ratio between of 80:20 and 50:50, more preferably between 75:25 and 65:35, even more preferably 60:40. Even more preferably, the oily core comprises or consists of medium-chain triglycerides having at least 90% w/w of Miglyol® 812N. Even more preferably, the oil consists of Miglyol® 812N.

In an embodiment (hereinafter embodiment #O2), the oily core consists of an oil and, optionally, one or more pharmacologically inactive components. Preferably, the embodiment further specifies that a pharmacologically inactive component is a substance that contributes solely to the physicochemical properties of the oily core and does not induce any receptor-mediated, enzymatic, metabolic, immunological or physiological effect in the subject at the concentrations used, wherein a substance is considered to induce a receptor-mediated, enzymatic, metabolic, immunological or physiological effect when, at the concentrations used in the oily core of the nanocapsule, it produces a statistically significant change in any of the following standard in vitro readouts: (i) specific receptor binding measured in receptor-binding assays; (ii) modulation of enzymatic turnover measured in enzyme-activity assays; (iii) alteration of intracellular signaling or transcriptional activity measured in cell-based functional assays; (iv) activation of immune cells measured by cytokine release or upregulation of immune-cell surface markers; or (v) modification of cellular metabolic activity measured in viability or metabolic-activity assays (e.g., ATP-based or redox-based assays). More preferably, the one or more pharmacologically inactive components are selected from the list consisting of: (i) triglycerides and mixed glycerides, selected from the group consisting of medium-chain triglycerides (MCT), long-chain triglycerides (LCT), caprylic/capric triglycerides, glyceryl tristearate, glyceryl tripalmitate, glyceryl trilaurate, glyceryl tristearate, and mixtures thereof; (ii) natural or semi-synthetic oils, selected from the group consisting of squalene, squalane, castor oil, sunflower oil, soybean oil, olive oil, coconut oil, jojoba oil, almond oil, palm oil, and mixtures thereof; (iii) fatty acids, selected from the group consisting of oleic acid, linoleic acid, linolenic acid, stearic acid, palmitic acid, lauric acid, myristic acid, behenic acid, and mixtures thereof; (iv) fatty alcohols and alcohol esters, selected from the group consisting of cetyl alcohol, stearyl alcohol, lauryl alcohol, myristyl alcohol, isopropyl myristate, isopropyl palmitate, ethyl oleate, glyceryl mono-, di-, or tri-esters, and mixtures thereof; (v) waxes and lipid-like solids, selected from the group consisting of beeswax, carnauba wax, candelilla wax, paraffin wax, microcrystalline wax, hydrogenated vegetable oils, ceramides, and mixtures thereof; (vi) lipophilic surfactants or stabilizers, selected from the group consisting of polyglyceryl esters (polyglyceryl-2 oleate, polyglyceryl-3 ricinoleate, polyglyceryl-6 distearate), sorbitan esters (Span 20, 40, 60, 80, 85), lecithins (soy, egg, hydrogenated), sucrose esters of fatty acids (sucrose laurate, sucrose stearate), PEG esters soluble in oil (PEG-15 stearate, PEG-20 sorbitan monostearate, PEG-30 castor oil), polyglyceryl-sorbitan esters, hydrogenated castor oil derivatives, and mixtures thereof; (vii) lipophilic antioxidants, selected from the group consisting of α-tocopherol, vitamin E acetate, butylated hydroxytoluene (BHT), butylated hydroxyanisole (BHA), ascorbyl palmitate, and mixtures thereof; (viii) lipophilic co-solvents or hydrophobic solubilizers, selected from the group consisting of vitamin A acetate, vitamin D3, cholesterol, coenzyme Q10, phytosterols, and mixtures thereof; (ix) hydrophobic polymers or modifiers of viscosity, selected from the group consisting of polyethylene, polycaprolactone (low molecular weight), polymethylsiloxane oils, ethylcellulose (solubilized in oil), and mixtures thereof; (x) inert mineral or inorganic lipophilic fillers, selected from the group consisting of colloidal silica (hydrophobic), talc (hydrophobic grade), magnesium stearate, and mixtures thereof; (xi) and mixtures thereof.

In an embodiment (hereinafter embodiment #O3), the oily core consists of an oil. Preferably, the oil is a pharmacologically inactive oil. Preferably, the pharmacologically inactive oil is selected from the list consisting of mineral oil, squalene oil, silicone oil, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C15 alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, fractionated coconut oil, castor oil, long-chain monoglycerides, long-chain diglycerides, long-chain triglycerides, medium-chain monoglycerides, medium-chain diglycerides, medium-chain triglycerides, oleic acid, linoleic acid, oleyl alcohol, isostearyl alcohol, ethyl oleate, synthetic or semi-synthetic derivatives and combinations thereof. More preferably, the pharmacologically inactive oil comprises or consists of medium-chain triglycerides (i.e. triglycerides with three fatty acids having an aliphatic tail of 6 to 12 carbon atoms). Even more preferably, the pharmacologically inactive oil comprises or consists of medium-chain triglycerides having a mixture of caprylic (C8) and capric (C10) triglycerides, wherein the mixture of caprylic (C8) and capric (C10) triglycerides is preferably in a w/w ratio between of 80:20 and 50:50, more preferably between 75:25 and 65:35, even more preferably 60:40. Even more preferably, the pharmacologically inactive oil comprises or consists of medium-chain triglycerides having at least 90% w/w of Miglyol® 812N. Even more preferably, the oil consists of Miglyol® 812N.

In an embodiment (hereinafter embodiment #O4), the oily core consists of an oil. Preferably, the oil is selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof. More preferably, the oil is selected from the list consisting of mineral oil, squalene oil, silicone oil, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C15 alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, fractionated coconut oil, castor oil, long-chain monoglycerides, long-chain diglycerides, long-chain triglycerides, medium-chain monoglycerides, medium-chain diglycerides, medium-chain triglycerides, oleic acid, linoleic acid, oleyl alcohol, isostearyl alcohol, ethyl oleate, synthetic or semi-synthetic derivatives and combinations thereof. Even more preferably, the oil comprises or consists of medium-chain triglycerides (i.e. triglycerides with three fatty acids having an aliphatic tail of 6 to 12 carbon atoms). Even more preferably, the oil comprises or consists of medium-chain triglycerides having a mixture of caprylic (C8) and capric (C10) triglycerides, wherein the mixture of caprylic (C8) and capric (C10) triglycerides is preferably in a w/w ratio between of 80:20 and 50:50, more preferably between 75:25 and 65:35, even more preferably 60:40. Even more preferably, the oil comprises or consists of medium-chain triglycerides having at least 90% w/w of Miglyol® 812N. Even more preferably, the oil comprises or consists of Miglyol® 812N.

The term “oil” refers to refers to any organic, lipid-based substance that is liquid or semi-solid at room temperature, primarily composed of hydrocarbons, fatty acids, esters, fatty alcohols, or their derivatives. It can originate from plant, animal, mineral, synthetic, or semi-synthetic sources, and is typically hydrophobic and immiscible with water, serving functions such as lubrication, emolliency, nutrient delivery, fragrance, or chemical solvency.

The term “pharmacologically inactive” refers to a substance that, at the concentrations used in the composition of the disclosure, does not exert any receptor-mediated, enzymatic, metabolic, immunological or physiological effect in the subject. Unless otherwise specified, a pharmacologically inactive component is a substance that contributes solely to the physicochemical properties of the oily core without triggering any biological activity. A substance is considered to exert such an effect when, at the concentrations present in the oily core of the nanocapsule, it produces a statistically significant change in any of the following standard in vitro readouts: (i) receptor binding; (ii) enzymatic turnover; (iii) intracellular signalling or transcriptional activity; (iv) immune-cell activation; or (v) cellular metabolic activity. Accordingly, a pharmacologically inactive substance is one that does not induce any such measurable changes under the conditions relevant to the nanocapsule system. Preferably, a component is considered pharmacologically inactive if it contributes solely to the physicochemical properties of the oily core and does not induce any receptor-mediated, enzymatic, metabolic, immunological or physiological effect in the subject at the concentrations used, wherein a substance is considered to induce a receptor-mediated, enzymatic, metabolic, immunological or physiological effect when, at the concentrations used in the oily core of the nanocapsule, it produces a statistically significant change in any of the following standard in vitro readouts: (i) specific receptor binding measured in receptor-binding assays; (ii) modulation of enzymatic turnover measured in enzyme-activity assays; (iii) alteration of intracellular signaling or transcriptional activity measured in cell-based functional assays; (iv) activation of immune cells measured by cytokine release or upregulation of immune-cell surface markers; or (v) modification of cellular metabolic activity measured in viability or metabolic-activity assays (e.g., ATP-based or redox-based assays).

In a preferred embodiment, the oil comprises or consists of at least 50%, 60%, 70%, 80% or 90% w/w of triglycerides with three fatty acids having an aliphatic tail of 6 to 12 carbon atoms. Preferably, the oil comprises or consists of at least 90% w/w of triglycerides with three fatty acids having an aliphatic tail of 6 to 12 carbon atoms. Even more preferably, the oil comprises or consists of at least 90% w/w of a mixture of caprylic (C8) and capric (C10) triglycerides. Preferably, the mixture of caprylic (C8) and capric (C10) triglycerides is in a w/w ratio between 80:20 and 50:50, more preferably between 75:25 and 65:35, even more preferably 60:40. Even more preferably, the oil comprises or consists of at least 90% w/w of Miglyol® 812N.

The term “long-chain” mono-, di-, or triglycerides refers to lipids composed of a glycerol molecule attached to one, two, or three long-chain fatty acids, typically containing 13 or more carbon atoms (commonly between C14 and C22). These lipids are prevalent in natural fats and oils and are widely used in the pharmaceutical, cosmetic, and food industries for their emulsifying and biocompatible properties. Examples of long-chain monoglycerides include glyceryl monolinoleate (C18:2), glyceryl monooleate (C18:1), and glyceryl monostearate (C18:0), with commercial products such as Maisine® CC (glyceryl monolinoleate) and Peceol™ (glyceryl monooleate). Long-chain diglycerides include glyceryl dioleate, glyceryl distearate, and glyceryl dilinoleate, with commercial examples like Capmul® (various diglycerides used as emulsifiers and solubilizers). Long-chain triglycerides include triolein (glyceryl trioleate, C18:1), trilinolein (glyceryl trilinoleate, C18:2), and tristearin (glyceryl tristearate, C18:0), and naturally occurring oils like soybean oil, corn oil, and safflower oil, commonly employed in emulsions and lipid nanoparticle formulations.

The term “medium-chain” mono-, di-, or triglycerides refers to lipids composed of a glycerol molecule attached to one, two, or three medium-chain fatty acids, typically containing 6 to 12 carbon atoms, such as caproic acid (C6), caprylic acid (C8), capric acid (C10), and lauric acid (C12). These lipids are known for their rapid digestion and absorption, making them useful in nutritional, pharmaceutical, and cosmetic applications. Examples of medium-chain monoglycerides include glyceryl monocaprylate (C8), glyceryl monocaprate (C10), and glyceryl monolaurate (C12), with commercial products such as Capmul® MCM (glyceryl monocaprylate) and Lauroglycol™ (glyceryl monolaurate), commonly used as solubilizers and emulsifiers. Examples of medium-chain diglycerides include glyceryl dicaprylate, glyceryl dicaprate, and glyceryl dilaurate, with commercial products like Captex® 200 (a blend of medium-chain diglycerides) and Capmul® 808DG (glyceryl dicaprylate/dicaprate), which are widely employed in lipid-based formulations for enhancing drug delivery and bioavailability. Natural sources rich in medium-chain triglycerides include coconut oil, palm kernel oil, and dairy fats such as butter and goat's milk. Some commercial examples include Miglyol® products (including Miglyol® 812N, Miglyol® 810 N, and Miglyol® 840 N) and Labrafac® products (such as Labrafac® Lipophile WL 1349 and Labrafac® CC), which essentially consist of a mixture of triglycerides of caprylic (C8) and capric (C10) acids.

The term “triglycerides with three fatty acids having an aliphatic tail of 6 to 12 carbon atoms”, generally referred to as medium-chain triglycerides (MCTs), refers to a type of glyceride molecule composed of a glycerol backbone linked through ester bonds to three fatty acids, each containing a straight-chain hydrocarbon tail with 6 to 12 carbon atoms. These fatty acids include caproic acid (C6:0), caprylic acid (C8:0), capric acid (C10:0), and lauric acid (C12:0). Examples of triglycerides formed from these fatty acids include tricaproin (from caproic acid), tricaprylin (from caprylic acid), tricaprin (from capric acid), and trilaurin (from lauric acid). Natural sources rich in these triglycerides include coconut oil, palm kernel oil, and dairy fats such as butter and goat's milk. Some commercial examples include Miglyol® products (including Miglyol® 812N, Miglyol® 810 N, and Miglyol® 840 N) and Labrafac® products (such as Labrafac Lipophile WL 1349 and Labrafac CC), which essentially consist of a mixture of triglycerides of caprylic (C8) and capric (C10) acids. As an expert in the field would recognize, several analytical techniques can be used to determine if an oil meets the characteristic of comprising triglycerides with three fatty acids having an aliphatic tail of 6 to 12 carbon atoms. First, gas chromatography (GC) is commonly employed to analyze the fatty acid composition of the oil. The oil sample is converted into fatty acid methyl esters (FAMEs), which are then separated based on their molecular size and structure. By comparing the retention times of the peaks to standards, it is possible to identify fatty acids with chain lengths ranging from 6 to 12 carbon atoms, such as caproic acid (C6), caprylic acid (C8), and lauric acid (C12). In combination with mass spectrometry (MS), which can be coupled with GC (GC-MS), the molecular weight and structure of the fatty acids can be further confirmed. High-performance liquid chromatography (HPLC) can also be used for a similar purpose, providing an alternative method for separating and identifying the fatty acids, although it is generally more suitable for less volatile compounds than those analyzed by GC. Additionally, nuclear magnetic resonance (NMR) spectroscopy can be applied to determine the exact structure of the fatty acids in the triglycerides by analyzing the chemical shifts and splitting patterns in the NMR spectra, providing detailed information about the carbon chain length and the presence of any unsaturation or branching in the fatty acids. Finally, the oil's composition can be further confirmed through fatty acid profiling, which involves a combination of these techniques to give a complete overview of the fatty acid content and verify the presence of triglycerides with the desired aliphatic chains. These techniques would also allow us to determine the specific percentage of each type of triglyceride in an oil.

The term “surfactant” refers to an amphiphilic molecule that reduces surface tension between two immiscible phases, typically a liquid and a solid or between two liquids. Surfactants possess both hydrophilic and hydrophobic components, allowing them to adsorb at interfaces and stabilize dispersions. Surfactants can be broadly categorized based on their solubility and ionic nature. In terms of solubility, surfactants are classified as either water-soluble or oil-soluble. Regarding ionic nature, they are grouped into anionic (negatively charged), cationic (positively charged), nonionic (no charge), and amphoteric or zwitterionic (both positive and negative charges) surfactants. In the context of the present disclosure, the term “surfactant” is also intended to cover emulsifying components with surfactant-like properties, such as phospholipids with surfactant-like properties. Examples of such emulsifying components that may not be traditionally considered as surfactants include phospholipids like Lipoid S75, which is derived from soybean lecithin and contains approximately 70% phosphatidylcholine. Other examples are natural emulsifiers such as egg yolk (rich in lecithin), certain proteins with emulsifying properties like casein or whey proteins, modified starches with amphiphilic characteristics, saponins (plant-derived compounds with surfactant-like properties), and certain polymers with both hydrophilic and hydrophobic segments, such as some cellulose derivatives. These components, while not classical surfactants, can function similarly in stabilizing emulsions, reducing interfacial tension, and facilitating the mixing of otherwise immiscible substances.

The examples set in the following section were performed using Lipoid S75 as a surfactant. Lipoid S75 is a soy lecithin-based phospholipid product, primarily composed of around 70% phosphatidylcholine. Lipoid S75 is a phospholipid-based compound that functions as an oil-soluble surfactant.

In a preferred embodiment, the surfactant is an oil-soluble surfactant.

The term “oil-soluble surfactant” refers to a surfactant that has a stronger affinity for oil than water due to its molecular structure, which typically features a larger hydrophobic portion compared to their hydrophilic component. They typically have a low Hydrophilic-Lipophilic Balance (HLB) value, generally between 3 and 6, which reflects their lipophilic nature. They are designed to stabilize water-in-oil emulsions and are widely used in formulations where the continuous phase is oil-based. Their hydrophobic portion interacts with the oil phase, while their limited hydrophilic components allow them to function effectively in non-aqueous systems. Examples of oil-soluble surfactants, including some oil-soluble emulsifying components with surfactant-like properties include: sorbitan esters—such as sorbitan monooleate (Span 80), sorbitan monolaurate (Span 20), sorbitan monostearate (Span 60), and sorbitan tristearate (Span 65)—, polyglycerol esters—such as polyglycerol polyricinoleate (PGPR) and polyglycerol oleate—, glyceryl esters (such as glyceryl monooleate and glyceryl monostearate), propylene glycol esters—such as propylene glycol monocaprylate (Capryol PGMC) and propylene glycol monolaurate—, polyglyceryl fatty acid esters—such as polyglyceryl-3 diisostearate and polyglyceryl-4 oleate—, sucrose esters (such as sucrose distearate and sucrose tristearate), fatty acid esters (such as isopropyl myristate, isopropyl palmitate, cetyl palmitate, and butyl stearate), dioctyl sodium sulfosuccinate (AOT), polyoxyethylene sorbitol hexaoleate, lanolin alcohols and derivatives (such as lanolin alcohol, acetylated lanolin alcohol, and hydrogenated lanolin), certain fatty acid salts with long hydrocarbon chains (between 12 and 22 carbon atoms)—such as calcium and magnesium salts of lauric acid (C12), myristic acid (C14), palmitic acid (C16), stearic acid (C18), and behenic acid (C22)—, and phospholipid-based compounds, such as Lipoid S75 (soy phospholipid with 70% phosphatidylcholine).

Preferably, the oil-soluble surfactant is selected from the list consisting of sorbitan esters, polyglycerol esters, glyceryl esters, propylene glycol esters, polyglyceryl fatty acid esters, sucrose esters, fatty acid esters, dioctyl sodium sulfosuccinate (AOT), polyoxyethylene sorbitol hexaoleate, lanolin alcohols and derivatives, fatty acid salts with hydrocarbon chains of 12 to 22 carbon atoms, and phospholipid-based compounds.

More preferably, the oil-soluble surfactant is a phospholipid-based compound.

Even more preferably, the oil-soluble surfactant is Lipoid S75 (soy phospholipid with 70% phosphatidylcholine).

The term “phospholipid-based compound” refers to a class of molecules derived from or structurally similar to phospholipids, which are amphiphilic lipids consisting of a hydrophilic head containing a phosphate group and two hydrophobic tails derived from fatty acids, typically joined by a glycerol backbone. These compounds retain the fundamental characteristics of phospholipids, including their ability to form bilayers and their importance in biological membranes. Phospholipid-based compounds may include natural phospholipids like phosphatidylcholine (lecithin), phosphatidylethanolamine, and phosphatidylserine, as well as modified or synthetic variants designed for specific applications in areas such as drug delivery, nanotechnology, and materials science.

In a preferred embodiment, the phospholipid-based compound is selected from the list consisting of lecithin, phosphatidylglycerol, phosphatidylserine, phosphatidylinositol, diphosphatidylglycerol, phosphatidic acid, phosphatidylcholine and phosphatidylethanolamine.

Preferably, the phospholipid-based compound is selected from the list consisting of Lipoid S75 (soy phospholipid with 70% phosphatidylcholine), hydrogenated lecithin, phosphatidylcholine, LIPOID S 45 (lecithin fraction with phosphatidylcholine content≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥90%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%).

More preferably, the phospholipid-based compound is Lipoid S75 (soy phospholipid with 70% phosphatidylcholine).

The nanocapsule system of the disclosure has an intrinsic prophylactic effect even in the absence of active ingredients. The examples set in the following section were performed using nanocapsules that are neither functionalized nor loaded with active ingredients. Instead, they possess an inherent prophylactic effect resulting solely from the combination of the nanocapsules' components.

In a preferred embodiment, the nanocapsule system comprises an empty oily core, devoid of active ingredients. Preferably, the nanocapsule system further comprises an empty surface layer, devoid of active ingredients.

The term “empty oily core” refers to the internal compartment of the nanocapsule that consists of oils or lipids but does not contain any active ingredient or bioactive compound. This means that the oily core functions primarily to stabilize the structure of the nanocapsule, providing a medium for encapsulation without any therapeutic, prophylactic or active compounds being loaded. To verify whether a nanocapsule has an empty oily core, methods such as Gas Chromatography (GC), Mass Spectrometry (MS), and Differential Scanning Calorimetry (DSC) are commonly used. GC can separate and identify individual components in the oily core, while MS provides detailed molecular information, confirming the absence of any active compounds. DSC measures the thermal properties of the nanocapsule and can help assess the nature of the oily core, ensuring no active ingredient is present. Additionally, an expert in the field will typically know whether a nanocapsule contains an active ingredient by consulting the product's datasheet, which specifies the components and composition used in the formulation. This allows the expert to verify the presence or absence of active ingredients based on the raw materials used.

The term “active ingredient” refers to a substance that provides a therapeutic, chemical, physical, or biological effect in the nanocapsule system. This includes drugs, vitamins, antioxidants, or other compounds with a functional impact. In the context of the present disclosure, the term “active ingredient” specifically excludes any components that are explicitly part of the nanocapsule system of the disclosure, regardless of whether they present inherent antimicrobial or protective properties, as well as any elements that may result from the combination of those components. Therefore, neither the chitosan surface layer, nor the oils that comprise the oily core, nor the surfactant are considered “active ingredients” in this context. To determine whether a nanocapsule contains an active ingredient, methods like High-Performance Liquid Chromatography (HPLC), UV-Vis Spectroscopy, and Liquid Chromatography-Mass Spectrometry (LC-MS) are typically employed. HPLC separates and quantifies the active compounds, while UV-Vis spectroscopy can detect specific active ingredients based on their characteristic absorbance. LC-MS, which combines the separation capabilities of liquid chromatography with the identification power of mass spectrometry, is especially useful for identifying and quantifying active ingredients. Experts in the field will typically know whether a nanocapsule has an active ingredient by referring to the datasheet of the materials used in the formulation. The datasheet will list any active compounds incorporated into the nanocapsules, allowing experts to confirm their presence or absence in the final product.

The term “empty surface layer” refers to the external coating of the nanocapsule that does not contain any active ingredients. This means the surface is free from any agents intended to provide therapeutic effects, regardless of the presence or absence of functional groups. To determine whether a nanocapsule has an empty surface layer, X-ray Photoelectron Spectroscopy (XPS) and Fourier Transform Infrared Spectroscopy (FTIR) can be used. XPS can analyze the elemental composition and detect the presence of any surface elements, while FTIR can provide insights into the molecular vibrations of the surface layer, confirming the absence of active ingredients. Experts in the field can also consult the datasheet of the nanocapsule components to verify whether any active ingredients were incorporated into the surface layer during production.

In a preferred embodiment, the nanocapsule system exhibits an intrinsic prophylactic effect in the absence of active ingredients.

The term “intrinsic” refers to a property or effect that is naturally inherent to the nanocapsule system itself—arising from the specific combination of its components—rather than being caused by an added active ingredient. It implies that the nanocapsule's prophylactic (protective) effect comes from the materials and structure of the nanocapsule alone, without relying on external therapeutic compounds. To assess whether a nanocapsule presents an intrinsic prophylactic effect in the absence of active ingredients, several approaches can be employed, including:
In vitro studies: Cell culture models using respiratory epithelial cells can be exposed to both the nanocapsule system and a respiratory pathogen. A reduction in pathogen attachment or cell infection—compared to a control group without nanocapsules—would suggest an intrinsic protective effect.
Animal models: In vivo experiments can assess whether subjects treated with the nanocapsules exhibit lower infection rates or less severe symptoms when exposed to respiratory pathogens—even without an active ingredient.
Biochemical analysis: Measuring inflammatory markers, cytokine production, or other immune responses after exposure to the nanocapsules can help determine whether they elicit a protective effect independent of active compounds.

An expert in the field would typically design these tests based on the nanocapsule's composition and refer to the datasheets of the materials involved to ensure no hidden active ingredients are contributing to the effect.

Regarding the physical and chemical properties of the nanocapsule system, the size of the nanocapsule plays an important role in determining its biological interactions, cellular uptake, and circulation time in the body, with smaller sizes generally promoting better cellular internalization and longer systemic circulation. Additionally, factors such as pH, the polydispersity index (Pdl), and zeta potential (Zpot) influence the stability, permeability, and release behavior of nanocapsules. pH affects the ionization state of the shell and core, altering interactions with the environment. Pdl refers to the distribution of particle sizes in a sample, with a lower Pdl indicating a more uniform size distribution and a higher Pdl reflecting a broader range of particle sizes. Zpot determines colloidal stability, with higher absolute values indicating better suspension stability and reduced aggregation.

In a preferred embodiment, the nanocapsules of the nanocapsule system of the disclosure have a mean diameter of between 1 and 999 nm, preferably between 50 and 500 nm, more preferably between 100 and 400 nm, even more preferably between 200 and 300 nm, even more preferably between 230 and 270 nm.

The term “mean diameter” refers to the diameter measured by means of the Dynamic Light Scattering (DLS) technique, which is defined by the hydrodynamic diameter of a sphere which scatters at the same speed as the particles being measured. The size of the nanocapsules is primarily influenced by the composition and formation conditions and can be measured using standard methods known by the person skilled in the art and described in the examples section.

In another preferred embodiment, the nanocapsules of the nanocapsule system of the disclosure have a pH of between 3.9 and 5.1, preferably between 4.1 and 4.9, more preferably between 4.3 and 4.7.

In another preferred embodiment, the nanocapsules of the nanocapsule system of the disclosure have a polydispersity index (Pdl) of between 0.05 and 0.35, preferably between 0.10 and 0.30, more preferably between 0.15 and 0.25.

In another preferred embodiment, the nanocapsules of the nanocapsule system of the disclosure have a zeta potential (Zpot) of between 5 mV and 45 mV, preferably between 15 mV and 35 mV.

In another preferred embodiment, the nanocapsules of the nanocapsule system of the disclosure have a pH of between 4.3 and 4.7, a polydispersity index (Pdl) of between 0.15 and 0.25, and a zeta potential (Zpot) of between 15 mV and 35 mV.

The nanocapsule system of the disclosure is for use in the prophylactic and/or therapeutic treatment of an infection.

The term ‘prophylactic treatment’ refers to a medical or therapeutic intervention intended to prevent the occurrence or onset of a disease or condition. Prophylactic treatment is preferably administered before any clinical symptoms appear, aiming to reduce the risk of developing the disease in the first place. In contrast, therapeutic treatment is designed to manage or cure a disease after clinical symptoms have already emerged. While prophylactic approaches focus on prevention, therapeutic treatments address an existing condition, aiming to alleviate symptoms, control progression, or achieve a cure. The key difference lies in timing and purpose: prophylactic treatment is preventive and proactive, preferably applied before the onset of clinical signs, whereas therapeutic treatment is reactive, initiated after symptoms are present.

The nanocapsule system of the disclosure is for use in the prophylactic and/or therapeutic treatment of an infection caused by a respiratory pathogen.

The term ‘infection caused by a respiratory pathogen’ refers to a disease or infection that occurs as a result of the invasion and multiplication of a respiratory pathogen (see definition below) that primarily targets and affects the respiratory system, which includes the nasal passages, throat, lungs, and airways. Examples of infections caused by respiratory pathogens include influenza (caused by the influenza virus), COVID-19 (caused by the coronavirus SARS-CoV-2), pneumonia (which can be caused by Streptococcus pneumoniae, Haemophilus influenzae, or viruses like respiratory syncytial virus), tuberculosis (caused by Mycobacterium tuberculosis), and fungal infections such as aspergillosis (caused by Aspergillus species).

The term “respiratory pathogen” refers to any microorganism that is capable of causing disease or infection in the respiratory system, including viruses (such as influenza, coronavirus, and respiratory syncytial virus), bacteria (such as Streptococcus pneumoniae and Mycobacterium tuberculosis), and fungi (such as Aspergillus species).

In a preferred embodiment, the respiratory pathogen is selected from the group consisting of a virus, a bacterium, or a fungus. Preferably, the respiratory pathogen is a respiratory virus. Preferably, the respiratory virus is selected from the group consisting of influenza virus, coronavirus, respiratory syncytial virus (RSV), parainfluenza virus, metapneumovirus, henipavirus, cytomegalovirus (CMV), varicella-zoster virus (VZV), herpes simplex virus (HSV), measles virus, mumps virus, rubella virus, rhinovirus (RV), adenovirus, bocavirus, enterovirus and any combination thereof.

In a more preferred embodiment, the respiratory pathogen is an enveloped respiratory virus. Preferably, the enveloped respiratory virus is selected from the group consisting of influenza virus, coronavirus, respiratory syncytial virus (RSV), parainfluenza virus, metapneumovirus, henipavirus, cytomegalovirus (CMV), varicella-zoster virus (VZV), herpes simplex virus (HSV), measles virus, mumps virus, rubella virus, and any combination thereof. More preferably, the enveloped respiratory virus is selected from the group consisting of influenza A virus, preferably H1 N1; coronavirus, preferably severe acute respiratory syndrome coronavirus 2 (SARS-CoV-2); and respiratory syncytial virus (RSV), preferably RSV subtype A. Even more preferably, the enveloped respiratory virus is influenza A virus, preferably H1 N1.

With regard to the embodiments concerning pathogens:
Preferably, the influenza virus is selected from the list consisting of influenza A virus (preferably selected from H1N1, H1N2, H2N2, H3N2, H5N1, H5N2, H5N5, H5N6, H6N1, H7N7, H7N9, H9N2, H10N7, H10N8, and H11N9), influenza B virus (preferably selected from Victoria lineage and Yamagata lineage), and influenza C virus (preferably selected from C/Taylor/1233/47, C/Mississippi/80, and C/Kanagawa/1/76 strains). More preferably, the influenza virus is an influenza A virus, preferably H1 N1.
Preferably, the coronavirus is selected from the list consisting of severe acute respiratory syndrome (SARS)-related coronavirus, preferably selected from SARS-CoV-1 and SARS-CoV-2, MERS-CoV, OC43, 229E, NL63, and HKU1. More preferably the coronavirus is of the genus Betacoronavirus, preferably selected from the list consisting of SARS-related coronavirus, preferably selected from SARS-CoV-1 and SARS-CoV-2, MERS-CoV, OC43 and HKU1. Even more preferably, the coronavirus is a SARS-related coronavirus, preferably selected from SARS-CoV-1 and SARS-CoV-2. Even more preferably, the coronavirus is the SARS-CoV-2.
Preferably, the respiratory syncytial virus (RSV) is selected from the list consisting of RSV subtype A and RSV subtype B. More preferably, the RSV is RSV subtype A.
Preferably, the parainfluenza virus is selected from the list consisting of parainfluenza virus types 1, 2, 3, and 4.
Preferably, the metapneumovirus is selected from the list consisting of metapneumovirus A1, A2, B1, and B2.
Preferably, the henipavirus is selected from the list consisting of Nipah virus and Hendra virus.
Preferably, the cytomegalovirus (CMV) is selected from the list consisting of Towne, AD169, and Merlin strains.
Preferably, the varicella-zoster virus (VZV) is selected from the list consisting of Oka, Ellen, and Varivax strains.
Preferably, the herpes simplex virus (HSV) is selected from the list consisting of HSV-1 and HSV-2.
Preferably, the measles virus is selected from the list consisting of Edmonston, Schwarz, Moraten, and AIK-C strains.
Preferably, the mumps virus is selected from the list consisting of Jeryl Lynn, Urabe, Rubini, and Leningrad-3 strains.
Preferably, the rubella virus is selected from the list consisting of RA27/3, HPV-77, and TO-336 strains.
Preferably, the rhinovirus (RV) is selected from the list consisting of RV-A (e.g., A16, A1, A2), RV-B (e.g., B14, B42), and RV-C (e.g., C2, C15, C41) strains.
Preferably, the adenovirus is selected from the list consisting of adenovirus species A (e.g., Ad12, Ad18), species B (e.g., Ad3, Ad7), species C (e.g., Ad1, Ad2, Ad5, Ad6), species D (e.g., Ad8, Ad19, Ad37), species E (e.g., Ad4), species F (e.g., Ad40, Ad41), and species G (e.g., Ad52) strains.
Preferably, the bocavirus is selected from the list consisting of bocavirus 1 (BOV1), BOV2, BOV3, and BOV4 strains.
Preferably, the enterovirus is selected from the list consisting of coxsackievirus A (e.g., CVA2, CVA4, CVA6, CVA16), coxsackievirus B (e.g., CVB1, CVB3, CVB5), echoviruses (e.g., Echo9, Echo11, Echo30), enterovirus A (e.g., EV-A71), enterovirus B (e.g., EV-B69), enterovirus C (e.g., poliovirus 1, 2, 3), and enterovirus D (e.g., EV-D68) strains.

The term “enveloped respiratory virus” refers to any virus that infects the human respiratory tract and possesses a lipid bilayer envelope derived from the host cell membrane. The presence of a lipid envelope can be confirmed by methods known in the art, including but not limited to: transmission electron microscopy to visualize the viral envelope, sensitivity to lipid solvents or detergents (e.g., ether or Triton X-100), and immunolabeling of viral membrane proteins associated with host-derived lipids. Enveloped respiratory viruses include, but are not limited to, influenza viruses (A, B, and C), coronaviruses (including SARS-CoV, MERS-CoV, and SARS-CoV-2), respiratory syncytial virus (RSV), parainfluenza viruses (types 1-4), metapneumoviruses, henipaviruses, cytomegalovirus (CMV), varicella-zoster virus (VZV), herpes simplex virus (HSV), measles virus, mumps virus, rubella virus, and any combination thereof.

The term “influenza virus” refers to a group of viruses belonging to the Orthomyxoviridae family, which are responsible for causing the infectious disease known as influenza, affecting the respiratory system. Influenza viruses are classified into several types, including A, B, and C, with Type A being the most common cause of seasonal flu epidemics. Influenza viruses possess a segmented, negative-sense RNA genome and a lipid bilayer envelope derived from the host cell membrane, which carries the viral surface glycoproteins hemagglutinin (HA) and neuraminidase (NA), responsible for viral entry and release, respectively. Influenza viruses possess a segmented, negative-sense RNA genome and a lipid bilayer envelope derived from the host cell membrane, which carries the viral surface glycoproteins hemagglutinin (HA) and neuraminidase (NA), responsible for viral entry and release, respectively. Influenza viruses include, but are not limited to: (i) influenza A virus, including subtypes H1 N1, H1 N2, H2N2, H3N2, H5N1, H5N2, H5N5, H5N6, H6N1, H7N7, H7N9, H9N2, H10N7, H10N8, and H11N9; (ii) influenza B virus, including Victoria lineage and Yamagata lineage; and (iii) influenza C virus, including C/Taylor/1233/47, C/Mississippi/80, and C/Kanagawa/1/76 strains.

The term “influenza A virus” refers to a subtype of the influenza virus that is responsible for most seasonal flu epidemics and pandemics. Influenza A viruses can infect humans as well as a variety of animals, including birds and pigs. This type of virus is further classified based on its surface proteins, hemagglutinin (H) and neuraminidase (N). Examples of influenza A virus subtypes include H1 N1, which caused the 2009 swine flu pandemic, and H3N2, which is commonly associated with seasonal flu outbreaks in humans.

The term “H1 N1 influenza” refers to a specific strain of the influenza A virus characterized by the H1 and N1 subtypes of hemagglutinin and neuraminidase, respectively. This strain gained widespread attention during the 2009 swine flu pandemic and is a common cause of seasonal influenza. The H1 N1 strain is closely related to the PR8 strain (A/Puerto Rico/8/34), which is a laboratory-adapted virus used in vaccine development and research. The PR8 strain is an early isolate of the H1 N1 virus and shares similar genetic and antigenic properties, serving as a model for studying influenza A virus infections and vaccine responses.

The term “influenza B virus” refers to a subtype of the influenza virus that primarily infects humans and is a significant cause of seasonal flu. Influenza B viruses are classified into lineages, mainly Victoria and Yamagata, which circulate in human populations. Influenza B viruses do not have subtypes like influenza A but can undergo genetic changes over time. Preferably, the influenza B virus is selected from the group consisting of Victoria lineage and Yamagata lineage viruses. Examples of Victoria lineage viruses include B/Brisbane/60/2008 and B/Massachusetts/02/2012, and examples of Yamagata lineage viruses include B/Phuket/3073/2013 and B/Texas/6/2011.

The term “influenza C virus” refers to a subtype of the influenza virus that infects humans and typically causes mild respiratory illness. Influenza C viruses are less common than influenza A and B viruses and are not classified into subtypes or lineages. Known influenza C virus strains are relatively stable but can circulate in human populations and occasionally cause outbreaks. Examples of influenza C virus strains include C/Ann Arbor/1/50 and C/Kanagawa/1/76.

The term “coronavirus” refers to any virus belonging to the family Coronaviridae, which consists of enveloped, positive-sense, single-stranded RNA viruses characterized by their crown-like (corona) appearance under electron microscopy due to spike (S) glycoproteins on their surface. Coronaviruses are classified into four genera: Alphacoronavirus, Betacoronavirus, Gammacoronavirus, and Deltacoronavirus. While Alphacoronavirus and Betacoronavirus primarily infect mammals, including humans, Gammacoronavirus and Deltacoronavirus mainly infect birds. Coronaviruses include, but are not limited to, severe acute respiratory syndrome coronavirus 1 (SARS-CoV-1), severe acute respiratory syndrome coronavirus 2 (SARS-CoV-2), Middle East respiratory syndrome coronavirus (MERS-CoV), HCoV-OC43, HCoV-229E, HCoV-NL63, and HCoV-HKU1.

The term “Betacoronavirus genus” refers to a virus belonging to the genus Betacoronavirus, which is part of the Coronaviridae family. Betacoronavirus includes several species of enveloped, positive-sense, single-stranded RNA viruses that primarily infect mammals and can cause respiratory, enteric, hepatic, and neurological diseases. Notable human-infecting Betacoronaviruses include SARS-CoV, SARS-CoV-2, MERS-CoV, HCoV-OC43, and HCoV-HKU1.

The term “Severe acute respiratory syndrome-related (SARS) coronavirus” refers to a virus belonging to the viral species Severe acute respiratory syndrome-related coronavirus within the genus Betacoronavirus and the family Coronaviridae. This species includes multiple coronaviruses capable of infecting humans and animals, including SARS-CoV1 and SARS-CoV-2, which are associated with severe respiratory diseases. These viruses share genetic and structural similarities and utilize the angiotensin-converting enzyme 2 (ACE2) receptor for host cell entry.

The term “SARS-CoV-1” refers to the specific strain of coronavirus identified as Severe Acute Respiratory Syndrome Coronavirus (SARS-CoV), which caused the 2002-2003 SARS outbreak. SARS-CoV belongs to the species Severe acute respiratory syndrome-related coronavirus within the genus Betacoronavirus and the family Coronaviridae. It is an enveloped, positive-sense, single-stranded RNA virus that primarily causes severe respiratory illness in humans.

The term “SARS-CoV-2” refers to the specific strain of coronavirus identified in 2019 as Severe Acute Respiratory Syndrome Coronavirus 2 (SARS-CoV-2), responsible for the COVID-19 pandemic. SARS-CoV-2 belongs to the species Severe acute respiratory syndrome-related coronavirus within the genus Betacoronavirus and the family Coronaviridae. It shares genetic and structural similarities with SARS-CoV but has distinct epidemiological and pathogenic characteristics. SARS-CoV-2 primarily spreads through respiratory droplets and utilizes the ACE2 receptor for host cell entry.

The term “respiratory syncytial virus (RSV)” refers to viruses of the species orthopneumovirus, genus Orthopneumovirus, family Paramyxoviridae, which infect the respiratory tract. RSV includes, but is not limited to, viruses classified into RSV subtype A and RSV subtype B. Examples of RSV strains include RSV A2 (ATCC VR-1540), RSV Long (ATCC VR-26), and RSV B1 (ATCC VR-1581).

The term “parainfluenza virus” refers to viruses of the genus respirovirus or Rubulavirus, family Paramyxoviridae, which infect the respiratory tract. Parainfluenza viruses include, but are not limited to, parainfluenza virus types 1, 2, 3, and 4, such as HPIV-1 (strain C35), HPIV-2 (strain Greer), HPIV-3 (strain C243), and HPIV-4 (strain SKPIV-4).

The term “metapneumovirus” refers to viruses of the species metapneumovirus, genus Metapneumovirus, family Paramyxoviridae, which infect the respiratory tract. Metapneumoviruses include, but are not limited to, metapneumovirus A1, A2, B1, and B2, such as subgroup A strain CAN97-83 and subgroup B strain NL/1/00.

The term “henipavirus” refers to viruses of the genus Henipavirus, family Paramyxoviridae, which infect mammals including humans. Henipaviruses include, but are not limited to, Nipah virus and Hendra virus.

The term “cytomegalovirus (CMV)” refers to viruses of the species human betaherpesvirus 5, family Herpesviridae, which infect the respiratory and other systems. CMV includes, but is not limited to, Towne, AD169, and Merlin strains.

The term “varicella-zoster virus (VZV)” refers to viruses of the species human alphaherpesvirus 3, family Herpesviridae, which infect the respiratory tract and skin. VZV includes, but is not limited to, Oka, Ellen, and Varivax strains.

The term “herpes simplex virus (HSV)” refers to viruses of the species human alphaherpesvirus 1 and 2, family Herpesviridae, which infect the respiratory tract and other tissues. HSV includes, but is not limited to, HSV-1 and HSV-2.

The term “measles virus” refers to viruses of the species measles virus, genus Morbillivirus, family Paramyxoviridae, which infect the respiratory tract. Measles viruses include, but are not limited to, Edmonston, Schwarz, Moraten, and AIK-C strains.

The term “mumps virus” refers to viruses of the species mumps virus, genus Rubulavirus, family Paramyxoviridae, which infect the respiratory tract. Mumps viruses include, but are not limited to, Jeryl Lynn, Urabe, Rubini, and Leningrad-3 strains.

The term “rubella virus” refers to viruses of the species rubella virus, genus Rubivirus, family Matonaviridae, which infect the respiratory tract. Rubella viruses include, but are not limited to, RA27/3, HPV-77, and TO-336 strains.

The nanocapsule system of the disclosure is for use in the prophylactic treatment and/or therapeutic of a subject in need.

The term “subject in need” refers to an individual who is at risk of contracting or developing a disease (in this case an infection caused by a respiratory pathogen) and who may benefit from prophylactic treatment to prevent such an infection. This includes individuals who are more vulnerable to respiratory infections due to factors such as age, pre-existing health conditions, immunocompromised states, or exposure to environments with a higher risk of pathogen transmission. Examples of subjects in need include the elderly, young children, individuals with chronic respiratory conditions (such as asthma or chronic obstructive pulmonary disease), individuals with weakened immune systems (e.g., those undergoing chemotherapy or living with HIV), healthcare workers exposed to respiratory pathogens, and individuals in high-risk environments such as nursing homes or hospitals.

In a preferred embodiment, the subject in need is a mammal, preferably a human.

The nanocapsule system of the disclosure is administered to the subject intranasally. As explained in the “Summary” section, the prophylactic effect of the nanocapsules results from their function as a physical barrier against the pathogen at the site of infection. To achieve this prophylactic effect, the nanocapsules must be administered in a manner that allows them to reach the sites of infection of respiratory pathogens, such as the nasal epithelium.

The term “intranasally” refers to a method of administering a substance through the nose, typically via sprays, drops, or aerosols. The substance is absorbed through the nasal mucosa, providing a route of entry into the body.

As mentioned above, the nanocapsule system of the examples set in the following section do not act as adjuvants, modulators, or delivery vehicles for other active compounds. Instead, they possess an inherent prophylactic and/or therapeutic effect.

In a preferred embodiment, the prophylactic and/or therapeutic treatment comprises or consists of preventing or reducing the respiratory pathogen from causing a symptomatic infection, preferably by preventing and/or reducing the interaction of the respiratory pathogen with the nasal epithelium by forming a protective barrier.

The term “preventing the respiratory pathogen from causing a symptomatic infection” refers to a process or intervention aimed at stopping or reducing the progression of a respiratory pathogen to a state where it produces clinical symptoms. This can include inhibiting the pathogen's replication, reducing its spread within the respiratory tract, or mitigating its ability to trigger an immune response that leads to symptoms such as coughing, sneezing, or inflammation. As an expert in the field would recognize, several methods can be employed individually or in combination to evaluate whether a prophylactic treatment effectively prevents a respiratory pathogen from causing a symptomatic infection:
In vitro cell culture assays: These involve exposing respiratory epithelial cells to the pathogen in the presence or absence of the prophylactic treatment. Researchers can measure the pathogen's replication rate and assess cell viability using techniques such as plaque assays, quantitative PCR (qPCR), or immunofluorescence microscopy to determine whether the treatment limits viral or bacterial proliferation, reducing the likelihood of symptom development.
Cytokine and inflammatory marker analysis: Since symptomatic infections often arise from the host's immune response to the pathogen, assays such as enzyme-linked immunosorbent assays (ELISA) or multiplex cytokine profiling can measure the levels of pro-inflammatory cytokines (e.g., IL-6, TNF-α). A reduction in these markers suggests that the treatment helps prevent the immune cascade leading to symptoms.
Animal models: In vivo studies using animals can assess whether the prophylactic treatment reduces both pathogen load and the onset of observable symptoms, such as respiratory distress or weight loss. Comparison of treated and untreated groups helps determine if the treatment prevents symptomatic infection.
Clinical symptom scoring: In human or animal studies, researchers can monitor for predefined clinical signs (e.g., nasal discharge, fever, or respiratory difficulty) alongside pathogen load assessments. A reduction or absence of such symptoms in the treatment group would indicate that the prophylactic approach is effective at preventing symptomatic infection.
Molecular assays: Polymerase chain reaction (PCR) or RNA sequencing can be used to quantify the pathogen's genetic material in respiratory tissues. Coupled with symptom monitoring, a reduction in both pathogen load and associated inflammatory gene expression supports the conclusion that the treatment prevents symptomatic infection.

The term “preventing and/or reducing the entry of the respiratory pathogen into the nasal epithelium” refers to a process or intervention aimed at stopping a respiratory pathogen (as defined above) from attaching to or penetrating the cells lining the nasal passages (the nasal epithelium). This can include blocking the pathogen's ability to bind to receptors or preventing its transport into the cells, thereby preventing infection. As an expert in the field would recognize, several methods can be employed individually or in combination to evaluate whether a prophylactic treatment effectively prevents a respiratory pathogen from entering the nasal epithelium:
In vitro cell culture assays: These involve exposing cultured nasal epithelial cells to the pathogen in the presence or absence of the prophylactic treatment. The ability of the pathogen to bind to the cells or invade them can be measured using techniques such as flow cytometry or immunofluorescence microscopy.
Binding inhibition assays: These assays assess whether the prophylactic treatment can block the pathogen from binding to specific receptors on the nasal epithelium. Techniques like enzyme-linked immunosorbent assays (ELISA) or surface plasmon resonance can help quantify the extent of binding inhibition.
Animal models: In vivo studies using animals can help evaluate the effect of the treatment on pathogen colonization in the nasal cavity. By comparing treated and untreated animals, researchers can determine if the treatment reduces pathogen presence in the nasal epithelium.
Molecular assays: Polymerase chain reaction (PCR) or RNA sequencing can be used to detect the presence of the pathogen in the nasal epithelium after treatment. A reduction in the pathogen's genetic material would indicate that the treatment has successfully blocked its entry.

The term “protective barrier” refers to a mechanism that prevents or reduces the entry of pathogens into the nasal epithelial cells. Possible protective barrier mechanisms may include: a physical barrier (a layer that physically blocks pathogens from reaching the nasal epithelium, such as a coating or film that prevents direct contact), a chemical barrier (substances that may alter the environment in a way that inhibits pathogens, such as altering pH or interfering with pathogen attachment), natural defense modulation (enhancement of the body's immune defenses, potentially by boosting mucus production or stimulating immune responses at the nasal surface), an enzymatic barrier (enzymes or substances that modulate enzymatic activity, enhancing the degradation or neutralization of pathogens before they can penetrate the nasal epithelium), or a hydration barrier (a moisture layer that could trap pathogens or facilitate their removal from the nasal passages).

In a preferred embodiment, the protective barrier is a physical barrier, such as a polymeric coating that prevents direct contact of pathogens with the nasal epithelium.

The term “physical barrier” refers to a layer that forms a continuous, uninterrupted coating over the nasal epithelium, preventing direct contact between pathogens and epithelial cells. As a person skilled in the art would recognize, this can be evaluated by visualizing the nanocapsule layer using electron microscopy to confirm the formation of a continuous coating over the epithelium and by testing pathogen adhesion reduction in vitro. An indicative threshold of effectiveness for a physical barrier would be a coating coverage of at least 95% of the epithelial surface, with pathogen adhesion reduced by at least 80% compared to untreated controls.

In another preferred embodiment, the protective barrier is a chemical barrier, such as a substance that alters the nasal pH to inhibit pathogen attachment or neutralize pathogens.

The term “chemical barrier” refers to a substance that alters the nasal pH or other chemical properties in a way that inhibits pathogen attachment or neutralizes pathogens. As a person skilled in the art would recognize, this can be determined by measuring pH changes after nanocapsule application and assessing pathogen viability or attachment in environments mimicking nasal conditions. An indicative threshold for a chemical barrier would be a pH shift of at least 0.5 units from the baseline nasal pH (typically around 6.0 to 6.5) and a reduction in pathogen viability or attachment by at least 75% in vitro.

In another preferred embodiment, the protective barrier enhances natural defense mechanisms, such as by stimulating increased mucus production or activating local immune cells.

The term “enhances natural defense mechanisms” refers to the stimulation of innate immune responses, including increased mucus production or activation of local immune cells, which helps trap and expel pathogens. As a person skilled in the art would recognize, this can be verified by analyzing mucus secretion levels and cytokine production in cell cultures or animal models treated with the nanocapsule system. An indicative threshold for enhancing natural defenses would be a 50% or greater increase in mucus production (e.g., mucin levels) and a statistically significant rise (p<0.05) in pro-inflammatory cytokines like IL-6 or IL-8 compared to baseline.

In another preferred embodiment, the protective barrier modulates enzymatic activity, such as by enhancing the production or activation of enzymes that break down pathogens before they penetrate the epithelium.

The term “modulates enzymatic activity” refers to the enhancement or activation of enzymes that degrade or neutralize pathogens before they penetrate the epithelium, such as lysozyme or proteases. As a person skilled in the art would recognize, this can be determined by measuring the activity levels of key enzymes in the nasal environment after nanocapsule administration. An indicative threshold for enzymatic modulation would be an increase in enzymatic activity (e.g., lysozyme or protease activity) by at least 60% relative to untreated controls, with corresponding pathogen degradation rates improved by at least 70%.

In another preferred embodiment, the protective barrier is a hydration barrier, for instance, a moisture layer that traps and flushes out pathogens from the nasal passages.

The term “hydration barrier” refers to a moisture layer that traps pathogens and facilitates their removal from the nasal passages through mucociliary clearance. As a person skilled in the art would recognize, this can be assessed by monitoring nasal surface hydration levels and evaluating the clearance rate of fluorescently labeled particles designed to simulate pathogen movement. An indicative threshold for a hydration barrier would be an increase in nasal surface hydration by at least 30% from baseline, along with a clearance rate of fluorescent particles improved by at least 50% compared to untreated conditions.

Other embodiments of the first aspect of the disclosure are set forth below. Moreover, all the embodiments of the first aspect of the disclosure apply herein:
In a particular embodiment (hereinafter “particular embodiment #1”) the nanocapsule system of the disclosure comprises or consists of: a) a surface layer comprising chitosan, b) an oily core, and c) a surfactant, the prophylactic and/or therapeutic treatment comprises or consists of preventing or reducing the respiratory pathogen from causing a symptomatic infection, and the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof.
   In a particular embodiment, particular embodiment #1 further mentions: the oil consists of at least 90% w/w of medium chain trigycerides (i.e. triglycerides with three fatty acids having an aliphatic tail of 6 to 12 carbon atoms).
   In a particular embodiment, particular embodiment #1 further mentions: the oil consists of at least 90% w/w of a mixture of caprylic (C8) and capric (C10) triglycerides.
   In a particular embodiment, particular embodiment #1 further mentions: the oil consists of at least 90% w/w of a mixture of caprylic (C8) and capric (C10) triglycerides in a w/w ratio between 80:20 and 50:50
   In a particular embodiment, particular embodiment #1 further mentions: the oil consists of at least 90% w/w of a mixture of caprylic (C8) and capric (C10) triglycerides in a w/w ratio between 75:25 and 65:35
   In a particular embodiment, particular embodiment #1 further mentions: the oil consists of at least 90% w/w of a mixture of caprylic (C8) and capric (C10) triglycerides in a w/w ratio of 60:40.
   In a particular embodiment, particular embodiment #1 further mentions: the oil consists of at least 90% w/w of Miglyol® 812N.
   In a particular embodiment, particular embodiment #1 further mentions: the oil consists of Miglyol® 812N.
In a particular embodiment (hereinafter “particular embodiment #2”) the nanocapsule system of the disclosure comprises or consists of: a) a surface layer comprising chitosan, b) an oily core, and c) a surfactant, the prophylactic and/or therapeutic treatment comprises or consists of preventing or reducing the respiratory pathogen from causing a symptomatic infection, and the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, the surfactant is an oil-soluble surfactant selected from the list consisting of sorbitan esters, polyglycerol esters, glyceryl esters, propylene glycol esters, polyglyceryl fatty acid esters, sucrose esters, fatty acid esters, dioctyl sodium sulfosuccinate (AOT), polyoxyethylene sorbitol hexaoleate, lanolin alcohols and derivatives, fatty acid salts with hydrocarbon chains of 12 to 22 carbon atoms, and phospholipid-based compounds.
   In a particular embodiment, particular embodiment #2 further mentions: the surfactant is a phospholipid-based compound selected from the list consisting of lecithin, phosphatidylglycerol, phosphatidylserine, phosphatidylinositol, diphosphatidylglycerol, phosphatidic acid, phosphatidylcholine and phosphatidylethanolamine.
   In a particular embodiment, particular embodiment #2 further mentions: the surfactant is a phospholipid-based compound selected from the list consisting of Lipoid S75 (soy phospholipid with 70% phosphatidylcholine), hydrogenated lecithin, phosphatidylcholine, LIPOID S 45 (lecithin fraction with phosphatidylcholine content≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥90%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%).
   In a particular embodiment, particular embodiment #2 further mentions: the surfactant is Lipoid S75.
In a particular embodiment (hereinafter “particular embodiment #3”) the nanocapsule system of the disclosure comprises or consists of: a) a surface layer comprising chitosan, b) an oily core, and c) a surfactant, the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, and the oil consists of at least 90% w/w of Miglyol® 812N and the surfactant is Lipoid S75.
   In a particular embodiment, particular embodiment #3 further mentions: the prophylactic and/or therapeutic treatment comprises or consists of preventing or reducing the respiratory pathogen from causing a symptomatic infection.
   In a particular embodiment, particular embodiment #3 further mentions: the prophylactic and/or therapeutic treatment comprises or consists of preventing or reducing the respiratory pathogen from causing a symptomatic infection, and the respiratory pathogen is a respiratory virus.
   In a particular embodiment, particular embodiment #3 further mentions: the prophylactic and/or therapeutic treatment comprises or consists of preventing or reducing the respiratory pathogen from causing a symptomatic infection, and the respiratory pathogen is a respiratory virus selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses (e.g. HCoV-229E, HCoV-NL63, HCoV-OC43 and HCoV-HKU1), bocavirus, and enteroviruses (e.g. coxsackieviruses and echoviruses).
   In a particular embodiment, particular embodiment #3 further mentions: the prophylactic treatment comprises or consists of preventing the respiratory pathogen from causing a symptomatic infection, and the respiratory pathogen is the influenza virus.
   In a particular embodiment, particular embodiment #3 further mentions: the prophylactic treatment comprises or consists of preventing the respiratory pathogen from causing a symptomatic infection, and the respiratory pathogen is an influenza A virus.
   In a particular embodiment, particular embodiment #3 further mentions: the prophylactic treatment comprises or consists of preventing the respiratory pathogen from causing a symptomatic infection, and the respiratory pathogen is the influenza virus.
In a particular embodiment (hereinafter “particular embodiment #4”) the nanocapsule system of the disclosure comprises or consists of: a) a surface layer comprising chitosan, b) an oily core, and c) a surfactant, the oily core consists of Miglyol® 812N and the surfactant is Lipoid S75.
   In a particular embodiment, particular embodiment #4 further mentions: the prophylactic treatment comprises or consists of preventing the respiratory pathogen from causing a symptomatic infection.
   In a particular embodiment, particular embodiment #4 further mentions: the prophylactic treatment comprises or consists of preventing the respiratory pathogen from causing a symptomatic infection the respiratory pathogen is a respiratory virus.
   In a particular embodiment, particular embodiment #4 further mentions: the prophylactic treatment comprises or consists of preventing the respiratory pathogen from causing a symptomatic infection, and the respiratory pathogen is a respiratory virus selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses (e.g. HCoV-229E, HCoV-NL63, HCoV-OC43 and HCoV-HKU1), bocavirus, and enteroviruses (e.g. coxsackieviruses and echoviruses).
   In a particular embodiment, particular embodiment #4 further mentions: the prophylactic treatment comprises or consists of preventing the respiratory pathogen from causing a symptomatic infection, and the respiratory pathogen is the influenza virus.
   In a particular embodiment, particular embodiment #4 further mentions: the prophylactic treatment comprises or consists of preventing the respiratory pathogen from causing a symptomatic infection, and the respiratory pathogen is an influenza A virus.
   In a particular embodiment, particular embodiment #4 further mentions: the prophylactic treatment comprises or consists of preventing the respiratory pathogen from causing a symptomatic infection, and the respiratory pathogen is the influenza virus.

Additionally, the first aspect of the disclosure also encompasses the following particular embodiments. Moreover, all the embodiments of the first aspect of the disclosure apply herein:
In a particular embodiment (hereinafter “#A.1”), the respiratory pathogen is an enveloped respiratory virus. Preferably, the enveloped respiratory virus is selected from the group consisting of influenza virus, coronavirus, respiratory syncytial virus (RSV), parainfluenza virus, metapneumovirus, henipavirus, cytomegalovirus (CMV), varicella-zoster virus (VZV), herpes simplex virus (HSV), measles virus, mumps virus, rubella virus, and any combination thereof. More preferably, the enveloped respiratory virus is selected from the group consisting of influenza A virus, preferably H1N1; coronavirus, preferably severe acute respiratory syndrome coronavirus 2 (SARS-CoV-2); and respiratory syncytial virus (RSV), preferably RSV subtype A. Even more preferably, the enveloped respiratory virus is influenza A virus, preferably H1 N1.
   In a particular embodiment (hereinafter “#A.1.1”), #A.1 further mentions that the nanocapsule system does not comprise the first excluded antigen (hereinafter “feature a”). In a particular embodiment, #A.1.1 further mentions embodiment #O1. In a particular embodiment, #A.1.1 further mentions embodiment #O2. In a particular embodiment, #A.1.1 further mentions embodiment #O3. In a particular embodiment, #A.1.1 further mentions embodiment #O4.
   In a particular embodiment (hereinafter “#A.1.2”), #A.1 further mentions that the nanocapsule system does not comprise the second excluded antigen (hereinafter “feature b”). In a particular embodiment, #A.1.2 further mentions embodiment #O1. In a particular embodiment, #A.1.2 further mentions embodiment #O2. In a particular embodiment, #A.1.2 further mentions embodiment #O3. In a particular embodiment, #A.1.2 further mentions embodiment #O4.
   In a particular embodiment (hereinafter “#A.1.3”), #A.1 further mentions that the nanocapsule system does not comprise the third excluded antigen (hereinafter “feature c”). In a particular embodiment, #A.1.3 further mentions embodiment #O1. In a particular embodiment, #A.1.3 further mentions embodiment #O2. In a particular embodiment, #A.1.3 further mentions embodiment #O3. In a particular embodiment, #A.1.3 further mentions embodiment #O4.
   In a particular embodiment (hereinafter “#A.1.4”), #A.1 further mentions that the nanocapsule system comprises or consist of a) a surface layer comprising chitosan, b) an oily core, and c) a surfactant (hereinafter “feature d”). In a particular embodiment, #A.1.4 further mentions embodiment #O1. In a particular embodiment, #A.1.4 further mentions embodiment #O2. In a particular embodiment, #A.1.4 further mentions embodiment #O3. In a particular embodiment, #A.1.4 further mentions embodiment #O4.
   In a particular embodiment (hereinafter “#A.1.5”), #A.1 further mentions that the surface layer does not comprise the first excluded antigen (hereinafter “feature e”). In a particular embodiment, #A.1.5 further mentions embodiment #O1. In a particular embodiment, #A.1.5 further mentions embodiment #O2. In a particular embodiment, #A.1.5 further mentions embodiment #O3. In a particular embodiment, #A.1.5 further mentions embodiment #O4.
   In a particular embodiment (hereinafter “#A.1.6”), #A.1 further mentions that the surface layer does not comprise the second excluded antigen (hereinafter “feature f”). In a particular embodiment, #A.1.6 further mentions embodiment #O1. In a particular embodiment, #A.1.6 further mentions embodiment #O2. In a particular embodiment, #A.1.6 further mentions embodiment #O3. In a particular embodiment, #A.1.6 further mentions embodiment #O4.
   In a particular embodiment (hereinafter “#A.1.7”), #A.1 further mentions that the surface layer does not comprise the third excluded antigen (hereinafter “feature g”). In a particular embodiment, #A.1.7 further mentions embodiment #O1. In a particular embodiment, #A.1.7 further mentions embodiment #O2. In a particular embodiment, #A.1.7 further mentions embodiment #O3. In a particular embodiment, #A.1.7 further mentions embodiment #O4.
   In a particular embodiment (hereinafter “#A.1.8”), #A.1 further mentions that the surface layer consists of chitosan and, optionally, one or more components, with the proviso that the one or more components do not comprise the first antigen of the disclosure (hereinafter “feature h”). In a particular embodiment, #A.1.8 further mentions embodiment #O1. In a particular embodiment, #A.1.8 further mentions embodiment #O2. In a particular embodiment, #A.1.8 further mentions embodiment #O3. In a particular embodiment, #A.1.8 further mentions embodiment #O4.
   In a particular embodiment (hereinafter “#A.1.9”), #A.1 further mentions that the surface layer consists of chitosan and, optionally, one or more components, with the proviso that the one or more components do not comprise the second antigen of the disclosure (hereinafter “feature i”). In a particular embodiment, #A.1.9 further mentions embodiment #O1. In a particular embodiment, #A.1.9 further mentions embodiment #O2. In a particular embodiment, #A.1.9 further mentions embodiment #O3. In a particular embodiment, #A.1.9 further mentions embodiment #O4.
   In a particular embodiment (hereinafter “#A.1.10”), #A.1 further mentions that the surface layer consists of chitosan and, optionally, one or more components, with the proviso that the one or more components do not comprise the third antigen of the disclosure (hereinafter “feature j”). In a particular embodiment, #A.1.10 further mentions embodiment #O1. In a particular embodiment, #A.1.10 further mentions embodiment #O2. In a particular embodiment, #A.1.10 further mentions embodiment #O3. In a particular embodiment, #A.1.10 further mentions embodiment #O4.
   In a particular embodiment (hereinafter “#A.1.11”), #A.1 further mentions that the surface layer consists of chitosan and, optionally, one or more components selected from the group consisting of: (i) a polysaccharide, selected from the group consisting of cellulose, hydroxypropyl methylcellulose, methylcellulose, ethylcellulose, carbomer, dextran, pullulan, hyaluronic acid, alginate, gellan gum, xanthan gum, inulin, pectin, and mixtures thereof; (ii) a polyol or sugar alcohol, selected from the group consisting of glycerol, sorbitol, mannitol, xylitol, erythritol, propylene glycol, and mixtures thereof; (iii) a synthetic polymer, selected from the group consisting of polyethylene glycol (PEG), polypropylene glycol (PPG), polyvinyl alcohol (PVA), polyvinylpyrrolidone (PVP), poloxamers, poly(2-ethyl-2-oxazoline), and mixtures thereof; (iii) a peptide or amino-acid-based compound lacking antigenic epitopes, selected from the group consisting of short neutral peptides (:20 aa), glycine, alanine, serine, valine, leucine, isoleucine, proline, hydroxyproline, and mixtures thereof; (iv) a lipid or fatty-acid-derived compound, selected from the group consisting of phosphatidylcholine, phosphatidylethanolamine, sphingomyelin, ceramides, mono-, di- and triglycerides, fatty acids (C8-C22), fatty alcohols (C8-C22), and mixtures thereof; (v) a zwitterionic or non-ionic surfactant-compatible stabilizer, selected from the group consisting of lecithin, polysorbates, sorbitan esters, sucrose esters, polyethylene glycol esters, polyglyceryl esters, and mixtures thereof; (vi) an inorganic or mineral component, selected from the group consisting of silica, colloidal silica, calcium phosphate, magnesium phosphate, titanium dioxide, and mixtures thereof; (vii) a plasticizer or flexibility-modifying agent, selected from the group consisting of triethyl citrate, citrate esters, sebacates, adipates, and mixtures thereof; (viii) a buffer or ionic excipient, selected from the group consisting of sodium chloride, potassium chloride, phosphate salts, citrate salts, acetate salts, and mixtures thereof; (ix) and mixtures thereof (hereinafter “feature k”). None of these components have antigens capable of eliciting an immune response against the enveloped respiratory virus. In a particular embodiment, #A.1.11 further mentions embodiment #O1. In a particular embodiment, #A.1.11 further mentions embodiment #O2. In a particular embodiment, #A.1.11 further mentions embodiment #O3. In a particular embodiment, #A.1.11 further mentions embodiment #O4.
   In a particular embodiment (hereinafter “#A.1.12”), #A.1 further mentions that the surface layer consists of chitosan (hereinafter “feature I”). In a particular embodiment, #A.1.12 further mentions embodiment #O1. In a particular embodiment, #A.1.12 further mentions embodiment #O2. In a particular embodiment, #A.1.12 further mentions embodiment #O3. In a particular embodiment, #A.1.12 further mentions embodiment #O4.
   In a particular embodiment (hereinafter “#A.1.13”), #A.1 further mentions embodiment that the nanocapsule system does not comprise the fourth excluded antigen (hereinafter “feature m”). In a particular embodiment, #A.1.13 further mentions embodiment #O1. In a particular embodiment, #A.1.13 further mentions embodiment #O2. In a particular embodiment, #A.1.13 further mentions embodiment #O3. In a particular embodiment, #A.1.13 further mentions embodiment #O4.
In a particular embodiment (hereinafter “#A.2”), the respiratory pathogen is an influenza virus. Preferably, the influenza virus is selected from the list consisting of influenza A virus (preferably selected from H1N1, H1N2, H2N2, H3N2, H5N1, H5N2, H5N5, H5N6, H6N1, H7N7, H7N9, H9N2, H10N7, H10N8, and H11N9), influenza B virus (preferably selected from Victoria lineage and Yamagata lineage), and influenza C virus (preferably selected from C/Taylor/1233/47, C/Mississippi/80, and C/Kanagawa/1/76 strains). More preferably, the influenza virus is an influenza A virus, preferably H1 N1.
   In a particular embodiment (hereinafter “#A.2.1”), #A.2 further mentions feature a. In a particular embodiment, #A.2.1 further mentions embodiment #O1. In a particular embodiment, #A.2.1 further mentions embodiment #O2. In a particular embodiment, #A.2.1 further mentions embodiment #O3. In a particular embodiment, #A.2.1 further mentions embodiment #O4.
   In a particular embodiment (hereinafter “#A.2.2”), #A.2 further mentions feature b. In a particular embodiment, #A.2.2 further mentions embodiment #O1. In a particular embodiment, #A.2.2 further mentions embodiment #O2. In a particular embodiment, #A.2.2 further mentions embodiment #O3. In a particular embodiment, #A.2.2 further mentions embodiment #O4.
   In a particular embodiment (hereinafter “#A.2.3”), #A.2 further mentions feature c. In a particular embodiment, #A.2.3 further mentions embodiment #O1. In a particular embodiment, #A.2.3 further mentions embodiment #O2. In a particular embodiment, #A.2.3 further mentions embodiment #O3. In a particular embodiment, #A.2.3 further mentions embodiment #O4.
   In a particular embodiment (hereinafter “#A.2.4”), #A.2 further mentions feature d. In a particular embodiment, #A.2.4 further mentions embodiment #O1. In a particular embodiment, #A.2.4 further mentions embodiment #O2. In a particular embodiment, #A.2.4 further mentions embodiment #O3. In a particular embodiment, #A.2.4 further mentions embodiment #O4.
   In a particular embodiment (hereinafter “#A.2.5”), #A.2 further mentions feature e. In a particular embodiment, #A.2.5 further mentions embodiment #O1. In a particular embodiment, #A.2.5 further mentions embodiment #O2. In a particular embodiment, #A.2.5 further mentions embodiment #O3. In a particular embodiment, #A.2.5 further mentions embodiment #O4.
   In a particular embodiment (hereinafter “#A.2.6”), #A.2 further mentions feature f. In a particular embodiment, #A.2.6 further mentions embodiment #O1. In a particular embodiment, #A.2.6 further mentions embodiment #O2. In a particular embodiment, #A.2.6 further mentions embodiment #O3. In a particular embodiment, #A.2.6 further mentions embodiment #O4.
   In a particular embodiment (hereinafter “#A.2.7”), #A.2 further mentions feature g. In a particular embodiment, #A.2.7 further mentions embodiment #O1. In a particular embodiment, #A.2.7 further mentions embodiment #O2. In a particular embodiment, #A.2.7 further mentions embodiment #O3. In a particular embodiment, #A.2.7 further mentions embodiment #O4.
   In a particular embodiment (hereinafter “#A.2.8”), #A.2 further mentions feature h. In a particular embodiment, #A.2.8 further mentions embodiment #O1. In a particular embodiment, #A.2.8 further mentions embodiment #O2. In a particular embodiment, #A.2.8 further mentions embodiment #O3. In a particular embodiment, #A.2.8 further mentions embodiment #O4.
   In a particular embodiment (hereinafter “#A.2.9”), #A.2 further mentions feature i. In a particular embodiment, #A.2.9 further mentions embodiment #O1. In a particular embodiment, #A.2.9 further mentions embodiment #O2. In a particular embodiment, #A.2.9 further mentions embodiment #O3. In a particular embodiment, #A.2.9 further mentions embodiment #O4.
   In a particular embodiment (hereinafter “#A.2.10”), #A.2 further mentions feature j. In a particular embodiment, #A.2.10 further mentions embodiment #O1. In a particular embodiment, #A.2.10 further mentions embodiment #O2. In a particular embodiment, #A.2.10 further mentions embodiment #O3. In a particular embodiment, #A.2.10 further mentions embodiment #O4.
   In a particular embodiment (hereinafter “#A.2.11”), #A.2 further mentions feature k. In a particular embodiment, #A.2.11 further mentions embodiment #O1. In a particular embodiment, #A.2.11 further mentions embodiment #O2. In a particular embodiment, #A.2.11 further mentions embodiment #O3. In a particular embodiment, #A.2.11 further mentions embodiment #O4.
   In a particular embodiment (hereinafter “#A.2.12”), #A.2 further mentions feature I. In a particular embodiment, #A.2.12 further mentions embodiment #O1. In a particular embodiment, #A.2.12 further mentions embodiment #O2. In a particular embodiment, #A.2.12 further mentions embodiment #O3. In a particular embodiment, #A.2.12 further mentions embodiment #O4.
   In a particular embodiment (hereinafter “#A.2.13”), #A.2 further mentions feature m. In a particular embodiment, #A.2.13 further mentions embodiment #O1. In a particular embodiment, #A.2.13 further mentions embodiment #O2. In a particular embodiment, #A.2.13 further mentions embodiment #O3. In a particular embodiment, #A.2.13 further mentions embodiment #O4.
In a particular embodiment (hereinafter “#A.3”), the respiratory pathogen is a coronavirus. Preferably, the coronavirus is selected from the list consisting of severe acute respiratory syndrome (SARS)-related coronavirus, preferably selected from SARS-CoV-1 and SARS-CoV-2, MERS-CoV, OC43, 229E, NL63, and HKU1. More preferably the coronavirus is of the genus Betacoronavirus, preferably selected from the list consisting of SARS-related coronavirus, preferably selected from SARS-CoV-1 and SARS-CoV-2, MERS-CoV, OC43 and HKU1. Even more preferably, the coronavirus is a SARS-related coronavirus, preferably selected from SARS-CoV-1 and SARS-CoV-2. Even more preferably, the coronavirus is the SARS-CoV-2.
   In a particular embodiment (hereinafter “#A.3.1”), #A.3 further mentions feature a. In a particular embodiment, #A.3.1 further mentions embodiment #O1. In a particular embodiment, #A.3.1 further mentions embodiment #O2. In a particular embodiment, #A.3.1 further mentions embodiment #O3. In a particular embodiment, #A.3.1 further mentions embodiment #O4.
   In a particular embodiment (hereinafter “#A.3.2”), #A.3 further mentions feature b. In a particular embodiment, #A.3.2 further mentions embodiment #O1. In a particular embodiment, #A.3.2 further mentions embodiment #O2. In a particular embodiment, #A.3.2 further mentions embodiment #O3. In a particular embodiment, #A.3.2 further mentions embodiment #O4.
   In a particular embodiment (hereinafter “#A.3.3”), #A.3 further mentions feature c. In a particular embodiment, #A.3.3 further mentions embodiment #O1. In a particular embodiment, #A.3.3 further mentions embodiment #O2. In a particular embodiment, #A.3.3 further mentions embodiment #O3. In a particular embodiment, #A.3.3 further mentions embodiment #O4.
   In a particular embodiment (hereinafter “#A.3.4”), #A.3 further mentions feature d. In a particular embodiment, #A.3.4 further mentions embodiment #O1. In a particular embodiment, #A.3.4 further mentions embodiment #O2. In a particular embodiment, #A.3.4 further mentions embodiment #O3. In a particular embodiment, #A.3.4 further mentions embodiment #O4.
   In a particular embodiment (hereinafter “#A.3.5”), #A.3 further mentions feature e. In a particular embodiment, #A.3.5 further mentions embodiment #O1. In a particular embodiment, #A.3.5 further mentions embodiment #O2. In a particular embodiment, #A.3.5 further mentions embodiment #O3. In a particular embodiment, #A.3.5 further mentions embodiment #O4.
   In a particular embodiment (hereinafter “#A.3.6”), #A.3 further mentions feature f. In a particular embodiment, #A.3.6 further mentions embodiment #O1. In a particular embodiment, #A.3.6 further mentions embodiment #O2. In a particular embodiment, #A.3.6 further mentions embodiment #O3. In a particular embodiment, #A.3.6 further mentions embodiment #O4.
   In a particular embodiment (hereinafter “#A.3.7”), #A.3 further mentions feature g. In a particular embodiment, #A.3.7 further mentions embodiment #O1. In a particular embodiment, #A.3.7 further mentions embodiment #O2. In a particular embodiment, #A.3.7 further mentions embodiment #O3. In a particular embodiment, #A.3.7 further mentions embodiment #O4.
   In a particular embodiment (hereinafter “#A.3.8”), #A.3 further mentions feature h. In a particular embodiment, #A.3.8 further mentions embodiment #O1. In a particular embodiment, #A.3.8 further mentions embodiment #O2. In a particular embodiment, #A.3.8 further mentions embodiment #O3. In a particular embodiment, #A.3.8 further mentions embodiment #O4.
   In a particular embodiment (hereinafter “#A.3.9”), #A.3 further mentions feature i. In a particular embodiment, #A.3.9 further mentions embodiment #O1. In a particular embodiment, #A.3.9 further mentions embodiment #O2. In a particular embodiment, #A.3.9 further mentions embodiment #O3. In a particular embodiment, #A.3.9 further mentions embodiment #O4.
   In a particular embodiment (hereinafter “#A.3.10”), #A.3 further mentions feature j. In a particular embodiment, #A.3.10 further mentions embodiment #O1. In a particular embodiment, #A.3.10 further mentions embodiment #O2. In a particular embodiment, #A.3.10 further mentions embodiment #O3. In a particular embodiment, #A.3.10 further mentions embodiment #O4.
   In a particular embodiment (hereinafter “#A.3.11”), #A.3 further mentions feature k. In a particular embodiment, #A.3.11 further mentions embodiment #O1. In a particular embodiment, #A.3.11 further mentions embodiment #O2. In a particular embodiment, #A.3.11 further mentions embodiment #O3. In a particular embodiment, #A.3.11 further mentions embodiment #O4.
   In a particular embodiment (hereinafter “#A.3.12”), #A.3 further mentions feature I. In a particular embodiment, #A.3.12 further mentions embodiment #O1. In a particular embodiment, #A.3.12 further mentions embodiment #O2. In a particular embodiment, #A.3.12 further mentions embodiment #O3. In a particular embodiment, #A.3.12 further mentions embodiment #O4.
   In a particular embodiment (hereinafter “#A.3.13”), #A.3 further mentions feature m. In a particular embodiment, #A.3.13 further mentions embodiment #O1. In a particular embodiment, #A.3.13 further mentions embodiment #O2. In a particular embodiment, #A.3.13 further mentions embodiment #O3. In a particular embodiment, #A.3.13 further mentions embodiment #O4.
In a particular embodiment (hereinafter “#A.4”), the respiratory virus is a respiratory syncytial virus (RSV). Preferably, the RSV is selected from the list consisting of RSV subtype A and RSV subtype B. More preferably, the RSV is RSV subtype A.
   In a particular embodiment (hereinafter “#A.4.1”), #A.4 further mentions feature a. In a particular embodiment, #A.4.1 further mentions embodiment #O1. In a particular embodiment, #A.4.1 further mentions embodiment #O2. In a particular embodiment, #A.4.1 further mentions embodiment #O3. In a particular embodiment, #A.4.1 further mentions embodiment #O4.
   In a particular embodiment (hereinafter “#A.4.2”), #A.4 further mentions feature b. In a particular embodiment, #A.4.2 further mentions embodiment #O1. In a particular embodiment, #A.4.2 further mentions embodiment #O2. In a particular embodiment, #A.4.2 further mentions embodiment #O3. In a particular embodiment, #A.4.2 further mentions embodiment #O4.
   In a particular embodiment (hereinafter “#A.4.3”), #A.4 further mentions feature c. In a particular embodiment, #A.4.3 further mentions embodiment #O1. In a particular embodiment, #A.4.3 further mentions embodiment #O2. In a particular embodiment, #A.4.3 further mentions embodiment #O3. In a particular embodiment, #A.4.3 further mentions embodiment #O4.
   In a particular embodiment (hereinafter “#A.4.4”), #A.4 further mentions feature d. In a particular embodiment, #A.4.4 further mentions embodiment #O1. In a particular embodiment, #A.4.4 further mentions embodiment #O2. In a particular embodiment, #A.4.4 further mentions embodiment #O3. In a particular embodiment, #A.4.4 further mentions embodiment #O4.
   In a particular embodiment (hereinafter “#A.4.5”), #A.4 further mentions feature e. In a particular embodiment, #A.4.5 further mentions embodiment #O1. In a particular embodiment, #A.4.5 further mentions embodiment #O2. In a particular embodiment, #A.4.5 further mentions embodiment #O3. In a particular embodiment, #A.4.5 further mentions embodiment #O4.
   In a particular embodiment (hereinafter “#A.4.6”), #A.4 further mentions feature f. In a particular embodiment, #A.4.6 further mentions embodiment #O1. In a particular embodiment, #A.4.6 further mentions embodiment #O2. In a particular embodiment, #A.4.6 further mentions embodiment #O3. In a particular embodiment, #A.4.6 further mentions embodiment #O4.
   In a particular embodiment (hereinafter “#A.4.7”), #A.4 further mentions feature g. In a particular embodiment, #A.4.7 further mentions embodiment #O1. In a particular embodiment, #A.4.7 further mentions embodiment #O2. In a particular embodiment, #A.4.7 further mentions embodiment #O3. In a particular embodiment, #A.4.7 further mentions embodiment #O4.
   In a particular embodiment (hereinafter “#A.4.8”), #A.4 further mentions feature h. In a particular embodiment, #A.4.8 further mentions embodiment #O1. In a particular embodiment, #A.4.8 further mentions embodiment #O2. In a particular embodiment, #A.4.8 further mentions embodiment #O3. In a particular embodiment, #A.4.8 further mentions embodiment #O4.
   In a particular embodiment (hereinafter “#A.4.9”), #A.4 further mentions feature i. In a particular embodiment, #A.4.9 further mentions embodiment #O1. In a particular embodiment, #A.4.9 further mentions embodiment #O2. In a particular embodiment, #A.4.9 further mentions embodiment #O3. In a particular embodiment, #A.4.9 further mentions embodiment #O4.
   In a particular embodiment (hereinafter “#A.4.10”), #A.4 further mentions feature j. In a particular embodiment, #A.4.10 further mentions embodiment #O1. In a particular embodiment, #A.4.10 further mentions embodiment #O2. In a particular embodiment, #A.4.10 further mentions embodiment #O3. In a particular embodiment, #A.4.10 further mentions embodiment #O4.
   In a particular embodiment (hereinafter “#A.4.11”), #A.4 further mentions feature k. In a particular embodiment, #A.4.11 further mentions embodiment #O1. In a particular embodiment, #A.4.11 further mentions embodiment #O2. In a particular embodiment, #A.4.11 further mentions embodiment #O3. In a particular embodiment, #A.4.11 further mentions embodiment #O4.
   In a particular embodiment (hereinafter “#A.4.12”), #A.2 further mentions feature I. In a particular embodiment, #A.2.12 further mentions embodiment #O1. In a particular embodiment, #A.4.12 further mentions embodiment #O2. In a particular embodiment, #A.4.12 further mentions embodiment #O3. In a particular embodiment, #A.4.12 further mentions embodiment #O4.
   In a particular embodiment (hereinafter “#A.4.13”), #A.4 further mentions feature m. In a particular embodiment, #A.4.13 further mentions embodiment #O1. In a particular embodiment, #A.4.13 further mentions embodiment #O2. In a particular embodiment, #A.4.13 further mentions embodiment #O3. In a particular embodiment, #A.4.13 further mentions embodiment #O4.

A second aspect of the disclosure refers to a pharmaceutical composition comprising the nanocapsule system of the disclosure (as defined in the first aspect of the disclosure) and, optionally, one or more pharmaceutically acceptable excipients, for use in the prophylactic and/or therapeutic treatment of an infection caused by a respiratory pathogen in a subject in need thereof, wherein the nanocapsule system is administered to the subject intranasally.

The nanocapsule system and the prophylactic treatment of an infection caused by a respiratory pathogen are the same as for the first aspects of the disclosure. Thus, all the embodiments mentioned in this regard for the first aspect apply herein.

The term “pharmaceutical composition” refers to a formulation that combines the nanocapsule system of the disclosure with other excipients or inactive substances to create a product intended for medical use. These excipients may include fillers, binders, stabilizers, preservatives, solvents, and other agents that help in the delivery, stability, and absorption of the nanocapsule or its components in the body. Pharmaceutical compositions can be in various forms, such as tablets, capsules, liquids, creams, or injectable solutions, depending on the intended route of administration and therapeutic purpose. In the context of the present disclosure the pharmaceutical composition may be specifically designed to be suitable for intranasal administration, ensuring proper delivery, absorption, and stability when applied to the nasal mucosa.

The term “pharmaceutically acceptable excipient” refers to a substance added to a drug formulation alongside the main medicinal component (in this case the nanocapsule system of the disclosure) to serve various purposes in the medicine's composition. These substances are typically inert and carefully evaluated for safety. They may aid in manufacturing, improve stability or bioavailability, enhance patient acceptability, or facilitate drug delivery. Excipients can include binders, disintegrants, fillers, preservatives, and lubricants, among others. While generally considered inactive, some excipients may have known effects in certain circumstances. In more complex drug delivery systems, such as nanocapsules, the distinction between excipients and active ingredients can become blurred, as all components of the nanoparticle may contribute to the efficacy and safety profiles of the final drug product.

The present disclosure also refers to the use of the nanocapsule system as defined in the first aspect of the disclosure for manufacturing a pharmaceutical composition for the prophylactic treatment of an infection caused by a respiratory pathogen in a subject in need. The “nanocapsule system”, “prophylactic treatment” and “infection caused by a respiratory pathogen” are the same as for the first aspect of the disclosure. Thus, all the embodiments mentioned in these regards for the first aspect apply herein.

The disclosure also refers to:

In view of the additional results suggesting that the prophylactic and antiviral effects of the chitosan-based nanocapsules apply to enveloped viruses, both respiratory and non-respiratory, we have included several additional aspects:

A third aspect of the disclosure refers to a nanocapsule system (hereinafter “nanocapsule system of the third aspect of the disclosure”), which comprises a surface layer comprising chitosan, for use in the prophylactic and/or therapeutic treatment of an infection caused by an enveloped virus in a subject in need thereof, wherein the nanocapsule system is administered at the site of viral entry or infection.

When applicable, all the embodiments mentioned for the first aspect of the disclosure apply herein.

The term “enveloped virus” refers to a group of viruses that possess a lipid bilayer envelope derived from the host cell membrane, which surrounds the viral capsid and contains viral glycoproteins involved in host cell attachment and entry. The presence of a lipid envelope can be confirmed by methods known in the art, including but not limited to: transmission electron microscopy to visualize the viral envelope, sensitivity to lipid solvents or detergents (e.g., ether or Triton X-100), and immunolabeling of viral membrane proteins associated with host-derived lipids. Enveloped respiratory viruses include, but are not limited to, influenza viruses (A, B, and C), coronaviruses (such as SARS-CoV, MERS-CoV, and SARS-CoV-2), respiratory syncytial virus (RSV), human parainfluenza viruses, human metapneumoviruses, herpesviruses, hepatitis B virus, hepatitis C virus, human immunodeficiency virus (HIV), and other viruses containing a lipid envelope.

In a preferred embodiment, the enveloped virus is selected from the list consisting of influenza virus, coronavirus, respiratory syncytial virus (RSV), parainfluenza virus, metapneumovirus, henipavirus, cytomegalovirus (CMV), varicella-zoster virus (VZV), herpes simplex virus (HSV), measles virus, mumps virus, rubella virus, human immunodeficiency virus (HIV), Ebola virus, Marburg virus, Lassa virus, Nipah virus, Hendra virus, hepatitis B virus (HBV), hepatitis C virus (HCV), dengue virus, Zika virus, West Nile virus, yellow fever virus, and any combination thereof. In a more preferred embodiment, the enveloped virus is selected from the list consisting of influenza virus, coronavirus, respiratory syncytial virus (RSV) and human immunodeficiency virus (HIV). In an even more preferred embodiment, the enveloped virus is HIV, preferably HIV-1.

With regard to embodiments concerning pathogens (moreover, the embodiments mentioned in this regard for the first aspect of the disclosure also apply herein):
Preferably, the human immunodeficiency virus (HIV) is selected from the list consisting of HIV-1 and HIV-2, preferably HIV-1.

The term “human immunodeficiency virus (HIV)” refers to viruses of the species Human immunodeficiency virus, genus Lentivirus, family Retroviridae, which infect cells of the immune system, including CD4+ T cells, macrophages, and dendritic cells. HIV includes, but is not limited to, viruses classified into HIV-1 and HIV-2. HIV-1 viruses include group M (subtypes A, B, C, D, F, G, H, J, and K), group N, group O, and group P, as well as circulating recombinant forms (CRFs). HIV-2 viruses include subtypes A, B, C, D, E, F, G, and H. Examples of specific HIV-1 strains include HIV-1 IIIB, HIV-1 NL4-3, HIV-1 Ba-L, and HIV-1 MN, while examples of HIV-2 strains include HIV-2 ROD and HIV-2 ST. HIV viruses may include any naturally occurring or laboratory-adapted variants, recombinants, or isolates derived therefrom, and the term is intended to cover all such variants without limitation.

The nanocapsule system of the third aspect of the disclosure is administered at the site of viral entry or infection. Such administration ensures that the nanocapsules can reach the anatomical locations where respiratory or other enveloped viruses initiate infection. As explained in the “Summary” section, the prophylactic effect of the nanocapsules arises from their ability to act as a physical barrier against the viral pathogen at its site of viral infection; therefore, administration must be carried out in a manner that allows the nanocapsules to access the relevant sites of infection. Accordingly, the appropriate route of administration will vary depending on the target virus and the anatomical site at which it establishes infection. For example, when prophylaxis is intended against viruses that initiate infection in the nasal epithelium or other regions of the upper respiratory tract, the formulation should be delivered through a route that facilitates access to these tissues, such as intranasal administration. Likewise, in the case of viruses that enter or replicate at other mucosal surfaces, the selected route of administration should be one that enables the nanocapsules to reach those specific anatomical locations.

Other embodiments of the third aspect of the disclosure are set forth below. Moreover, all the embodiments of the third aspect of the disclosure apply herein:
In a particular embodiment, the nanocapsule system comprises feature a. In a particular embodiment, the embodiment further mentions embodiment #O1, #O2, #O3 or #O4.
In a particular embodiment, the nanocapsule system comprises feature b. In a particular embodiment, the embodiment further mentions embodiment #O1, #O2, #O3 or #O4.
In a particular embodiment, the nanocapsule system comprises feature c. In a particular embodiment, the embodiment further mentions embodiment #O1, #O2, #O3 or #O4.
In a particular embodiment, the nanocapsule system comprises feature d. In a particular embodiment, the embodiment further mentions embodiment #O1, #O2, #O3 or #O4.
In a particular embodiment, the nanocapsule system comprises feature e. In a particular embodiment, the embodiment further mentions embodiment #O1, #O2, #O3 or #O4.
In a particular embodiment, the nanocapsule system comprises feature f. In a particular embodiment, the embodiment further mentions embodiment #O1, #O2, #O3 or #O4.
In a particular embodiment, the nanocapsule system comprises feature g. In a particular embodiment, the embodiment further mentions embodiment #O1, #O2, #O3 or #O4.
In a particular embodiment, the nanocapsule system comprises feature h. In a particular embodiment, the embodiment further mentions embodiment #O1, #O2, #O3 or #O4.
In a particular embodiment, the nanocapsule system comprises feature i. In a particular embodiment, the embodiment further mentions embodiment #O1, #O2, #O3 or #O4.
In a particular embodiment, the nanocapsule system comprises feature j. In a particular embodiment, the embodiment further mentions embodiment #O1, #O2, #O3 or #O4.
In a particular embodiment, the nanocapsule system comprises feature k. In a particular embodiment, the embodiment further mentions embodiment #O1, #O2, #O3 or #O4.
In a particular embodiment, the nanocapsule system comprises feature I. In a particular embodiment, the embodiment further mentions embodiment #O1, #O2, #O3 or #O4.
In a particular embodiment, the nanocapsule system comprises feature m. In a particular embodiment, the embodiment further mentions embodiment #O1, #O2, #O3 or #O4.
In a particular embodiment (hereinafter “#A.5”), the enveloped virus is the human immunodeficiency virus (HIV).
   In a particular embodiment (hereinafter “#A.5.1”), #A.5 further mentions feature a. In a particular embodiment, #A.5.1 further mentions embodiment #O1. In a particular embodiment, #A.5.1 further mentions embodiment #O2. In a particular embodiment, #A.5.1 further mentions embodiment #O3. In a particular embodiment, #A.5.1 further mentions embodiment #O4.
   In a particular embodiment (hereinafter “#A.5.2”), #A.5 further mentions feature b. In a particular embodiment, #A.5.2 further mentions embodiment #O1. In a particular embodiment, #A.5.2 further mentions embodiment #O2. In a particular embodiment, #A.5.2 further mentions embodiment #O3. In a particular embodiment, #A.5.2 further mentions embodiment #O4.
   In a particular embodiment (hereinafter “#A.5.3”), #A.5 further mentions feature c. In a particular embodiment, #A.5.3 further mentions embodiment #O1. In a particular embodiment, #A.5.3 further mentions embodiment #O2. In a particular embodiment, #A.5.3 further mentions embodiment #O3. In a particular embodiment, #A.5.3 further mentions embodiment #O4.
   In a particular embodiment (hereinafter “#A.5.4”), #A.5 further mentions feature d. In a particular embodiment, #A.5.4 further mentions embodiment #O1. In a particular embodiment, #A.5.4 further mentions embodiment #O2. In a particular embodiment, #A.5.4 further mentions embodiment #O3. In a particular embodiment, #A.5.4 further mentions embodiment #O4.
   In a particular embodiment (hereinafter “#A.5.5”), #A.5 further mentions feature e. In a particular embodiment, #A.5.5 further mentions embodiment #O1. In a particular embodiment, #A.5.5 further mentions embodiment #O2. In a particular embodiment, #A.5.5 further mentions embodiment #O3. In a particular embodiment, #A.5.5 further mentions embodiment #O4.
   In a particular embodiment (hereinafter “#A.5.6”), #A.5 further mentions feature f. In a particular embodiment, #A.5.6 further mentions embodiment #O1. In a particular embodiment, #A.5.6 further mentions embodiment #O2. In a particular embodiment, #A.5.6 further mentions embodiment #O3. In a particular embodiment, #A.5.6 further mentions embodiment #O4.
   In a particular embodiment (hereinafter “#A.5.7”), #A.5 further mentions feature g. In a particular embodiment, #A.5.7 further mentions embodiment #O1. In a particular embodiment, #A.5.7 further mentions embodiment #O2. In a particular embodiment, #A.5.7 further mentions embodiment #O3. In a particular embodiment, #A.5.7 further mentions embodiment #O4.
   In a particular embodiment (hereinafter “#A.5.8”), #A.5 further mentions feature h. In a particular embodiment, #A.5.8 further mentions embodiment #O1. In a particular embodiment, #A.5.8 further mentions embodiment #O2. In a particular embodiment, #A.5.8 further mentions embodiment #O3. In a particular embodiment, #A.5.8 further mentions embodiment #O4.
   In a particular embodiment (hereinafter “#A.5.9”), #A.5 further mentions feature i. In a particular embodiment, #A.5.9 further mentions embodiment #O1. In a particular embodiment, #A.5.9 further mentions embodiment #O2. In a particular embodiment, #A.5.9 further mentions embodiment #O3. In a particular embodiment, #A.5.9 further mentions embodiment #O4.
   In a particular embodiment (hereinafter “#A.5.10”), #A.5 further mentions feature j. In a particular embodiment, #A.5.10 further mentions embodiment #O1. In a particular embodiment, #A.5.10 further mentions embodiment #O2. In a particular embodiment, #A.5.10 further mentions embodiment #O3. In a particular embodiment, #A.5.10 further mentions embodiment #O4.
   In a particular embodiment (hereinafter “#A.5.11”), #A.5 further mentions feature k. In a particular embodiment, #A.5.11 further mentions embodiment #O1. In a particular embodiment, #A.5.11 further mentions embodiment #O2. In a particular embodiment, #A.5.11 further mentions embodiment #O3. In a particular embodiment, #A.5.11 further mentions embodiment #O4.
   In a particular embodiment (hereinafter “#A.5.12”), #A.5 further mentions feature I. In a particular embodiment, #A.5.12 further mentions embodiment #O1. In a particular embodiment, #A.5.12 further mentions embodiment #O2. In a particular embodiment, #A.5.12 further mentions embodiment #O3. In a particular embodiment, #A.5.12 further mentions embodiment #O4.
   In a particular embodiment (hereinafter “#A.5.13”), #A.5 further mentions feature m. In a particular embodiment, #A.5.13 further mentions embodiment #O1. In a particular embodiment, #A.5.13 further mentions embodiment #O2. In a particular embodiment, #A.5.13 further mentions embodiment #O3. In a particular embodiment, #A.5.13 further mentions embodiment #O4.

A fourth aspect of the disclosure refers to a pharmaceutical composition comprising the nanocapsule system of the third aspect of the disclosure and, optionally, one or more pharmaceutically acceptable excipients, for use in the prophylactic and/or therapeutic treatment of an infection caused by an enveloped virus in a subject in need thereof, wherein the nanocapsule system is administered at the site of viral entry or infection.

The nanocapsule system and the prophylactic treatment of an infection caused by an enveloped virus are the same as for the third aspect of the disclosure. Thus, all the embodiments mentioned in this regard for the third aspect apply herein.

The present disclosure also refers to the use of the nanocapsule system of the third aspect of the disclosure for manufacturing a pharmaceutical composition for the prophylactic treatment of an infection caused by a respiratory pathogen in a subject in need. The “nanocapsule system”, “prophylactic treatment” and “infection caused by a respiratory pathogen” are the same as for the third aspect of the disclosure. Thus, all the embodiments mentioned in these regards for the third aspect apply herein.

The disclosure also provides the following items:
1. A nanocapsule system, which comprises:
   a) a surface layer comprising chitosan,
   b) an oily core, and
   c) a surfactant,
   for use in the prophylactic and/or therapeutic treatment of an infection caused by a respiratory pathogen in a subject in need thereof, wherein the nanocapsule system is administered to the subject intranasally.
2. The nanocapsule system according to item 1, wherein the prophylactic treatment comprises preventing the respiratory pathogen from causing a symptomatic infection.
3. The nanocapsule system according to any of the item 1 or 2, for use in the prophylactic treatment of an infection caused by a respiratory virus.
4. The nanocapsule system according to item 3, wherein the respiratory virus is selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses, bocavirus, and enteroviruses.
5. The nanocapsule system according to item 4, wherein the respiratory virus is the influenza virus.
6. The nanocapsule system according to any of the items 1 to 5, wherein the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof.
7. The nanocapsule system according to any of the items 1 to 6, wherein the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, and wherein the prophylactic treatment comprises preventing the respiratory pathogen from causing a symptomatic infection.
8. The nanocapsule system according to any of the items 6 or 7, wherein the oil consists of at least 90% w/w of triglycerides with three fatty acids having an aliphatic tail of 6 to 12 carbon atoms.
9. The nanocapsule system according to any of the items 6 to 8, wherein the oil consists of at least 90% w/w of a mixture of caprylic (C8) and capric (C10) triglycerides.
10. The nanocapsule system according to any of the items 6 to 9, wherein the oil consists of at least 90% w/w of a mixture of caprylic (C8) and capric (C10) triglycerides in a w/w ratio between 80:20 and 50:50.
11. The nanocapsule system according to any of the items 1 to 10, wherein the surfactant is an oil-soluble surfactant.
12. The nanocapsule system according to item 11, the oil-soluble surfactant is a phospholipid-based compound selected from the list consisting of lecithin, phosphatidylglycerol, phosphatidylserine, phosphatidylinositol, diphosphatidylglycerol, phosphatidic acid, phosphatidylcholine and phosphatidylethanolamine.
13. The nanocapsule system according to item 12, wherein the phospholipid-based compound is selected from the list consisting of Lipoid S75 (soy phospholipid with 70% phosphatidylcholine), hydrogenated lecithin, phosphatidylcholine, LIPOID S 45 (lecithin fraction with phosphatidylcholine content≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥90%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%).
14. The nanocapsule system according to item 13, wherein the phospholipid-based compound is Lipoid S75 (soy phospholipid with 70% phosphatidylcholine).
15. The nanocapsule system according to any of the item 1 to 14, wherein the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof, wherein the oil consists of at least 90% w/w of a mixture of caprylic (C8) and capric (C10) triglycerides in a w/w ratio between 80:20 and 50:50, wherein the surfactant is Lipoid S75 (soy phospholipid with 70% phosphatidylcholine), for use in the prophylactic treatment of an infection caused by the influenza virus.

The disclosure also provides the following items:

For clarity, the numbering of the following items restarts from 1. Any reference to an item number in this section refers to the corresponding item of the present list, and not to those of the previous section.
1. A nanocapsule system, which comprises:
   a) a surface layer consisting of chitosan,
   b) an oily core, and
   c) a surfactant,
   for use in the prophylactic treatment of an infection caused by a respiratory virus in a subject in need thereof, wherein the nanocapsule system is administered to the subject intranasally.
2. The nanocapsule system according to item 1, wherein the respiratory virus is selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses, bocavirus, and enteroviruses.
3. The nanocapsule system according to item 2, wherein the respiratory virus is the influenza virus.
4. The nanocapsule system according to item 2, wherein the respiratory virus is the severe acute respiratory syndrome coronavirus 2 (SARS-CoV-2).
5. The nanocapsule system according to item 1, wherein the nanocapsule system consists of:
   a) a surface layer consisting of chitosan,
   b) an oily core, and
   c) a surfactant.
6. The nanocapsule system according to item 5, wherein the respiratory virus is the influenza virus.
7. The nanocapsule system according to item 5, wherein the respiratory virus is the respiratory virus is the severe acute respiratory syndrome coronavirus 2 (SARS-CoV-2).
8. The nanocapsule system according to any of the items 5 to 7, wherein the oily core consists of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof.
9. The nanocapsule system according to item 8, wherein the oil is a medium-chain triglyceride having at least 90% w/w of triglycerides with three fatty acids having an aliphatic tail of 6 to 12 carbon atoms.
10. The nanocapsule system according to item 9, wherein the oil is a medium-chain triglyceride having at least 90% w/w of a mixture of caprylic (C8) and capric (C10) triglycerides.
11. The nanocapsule system according to any of the items 1 to 10, wherein the surfactant is an oil-soluble surfactant.
12. The nanocapsule system according to item 11, the oil-soluble surfactant is a phospholipid-based compound selected from the list consisting of lecithin, phosphatidylglycerol, phosphatidylserine, phosphatidylinositol, diphosphatidylglycerol, phosphatidic acid, phosphatidylcholine and phosphatidylethanolamine.
13. The nanocapsule system according to item 12, wherein the phospholipid-based compound is selected from the list consisting of Lipoid S75 (soy phospholipid with 70% phosphatidylcholine), hydrogenated lecithin, phosphatidylcholine, LIPOID S 45 (lecithin fraction with phosphatidylcholine content≥45%), PHOSPHOLIPON® 90 G (phosphatidylcholine content≥94%), LIPOID P 75 (phospholipids with phosphatidylcholine content≥70%), LIPOID P 100 (phosphatidylcholine content≥90%), LIPOID H 20 (lecithin fraction with phosphatidylcholine content≥20%), LIPOID H 100 (phosphatidylcholine content≥94%), PHOSPHOLIPON® 80 H (hydrogenated phospholipids with phosphatidylcholine content≥70%), PHOSPHOLIPON® 90 H (hydrogenated phosphatidylcholine content≥90%), LIPOID P 75-3 (hydrogenated phospholipids with phosphatidylcholine content≥62%), LIPOID P 100-3 (hydrogenated phosphatidylcholine content≥90%), and LIPOID H 100-3 (hydrogenated phosphatidylcholine content≥90%).
14. The nanocapsule system according to item 6, wherein the respiratory virus is the influenza virus and the nanocapsule system consists of:
   a) a surface layer consisting of chitosan;
   b) an oily core consisting of an oil selected from the list consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof; and
   c) a surfactant, wherein the surfactant is an oil-soluble surfactant.
15. The nanocapsule system according to item 7, wherein the respiratory virus is the severe acute respiratory syndrome coronavirus 2 (SARS-CoV-2); and the nanocapsule system is as defined in item 14.

### EXAMPLES

The present disclosure is illustrated by means of the Examples set forth below without the intention of limiting its scope of protection.

### Example 1. Materials and Methods

### 1.1. Preparation of Solutions

Notes: let all components reach RT before use (approximately 20 minutes).

1.1.1. Solution of Chitosan HCl in Ultrapure Water (0.25 mg/mL)

Weight 5 mg of Chitosan HCl in a disposable sterile ultra-clear polypropylene container (50 mL), equip with a stirring magnet, measure 20 mL of ultrapure water using the 25 mL graduated cylinder and add them, cap the container, and stir at RT for at least 60 minutes using the multipoint magnetic stirrer. Use within the preparation day.

Once perfectly dissolved, 10 mL of chitosan solution should be filtered through a 0.22 μm filter. The solution is aspirated employing a needle and a 10 mL syringe. Then, it is pushed out across the filter. The filtered solution should be kept in a new disposable sterile ultra-clear polypropylene container.

1.1.2. Solution of Lipoid S75 in Ethanol (40 mg/mL)

Weight 40 mg of Lipoid S75 in a 10 mL glass vial (ideally transparent), equip with a stirring magnet, add 1 mL of ethanol using an automatic pipette, cap the vial, and stir at RT for at least 30 minutes using the multipoint magnetic stirrer. If needed, vortex the mixture to ensure complete homogenization. Use within the preparation day.

### 1.2. Emulsification

### 1.2.1. Preparation of the Aqueous Phase (2.7 mL)

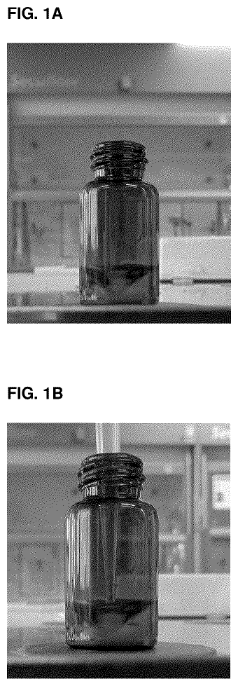

Measure 2.7 mL of the chitosan solution using the 1000-5000 μL automatic pipette, pour them onto a 10 mL amber glass vial equipped with a stirring magnet, and stir at 700 rpm or until a stable vortex is achieved, using the multipoint magnetic stirrer (FIG. 1A).

### 1.2.2. Preparation of the Organic Phase 2× (0.678 mL)

In a 1.5 mL amber Eppendorf tube, add 300 μL of Lipoid S75 solution (40 mg/mL), 78 μL of Miglyol 812N and 300 μL of ethanol. Close the cap. Vortex briefly. Use immediately.

### 1.2.3. Preparation of the Emulsion (3.039 mL)

Pour 339 μL of the organic phase at once over the center of the vortex of the aqueous phase using a 100-1000 μL automatic pipette, as closest to the bottom as possible without touching the aqueous solution with the pipette tip (FIG. 1B).

Mixture turns white immediately. Cap and stir for 5 minutes, then let rest for another 5 minutes.

### Example 2. Formulations with Other Chitosans

In the initial phase of formulation development, the use of different chitosans was studied (Table 2). We finally selected chitosan HCl (HMC) for the subsequent phases of qualitative and quantitative composition optimization. The exact composition of the chitosan nanocapsules (hereinafter CS nanocapsules) is shown in Table 3.

TABLE 2
Characteristics of formulations containing chitosan as a polymer.
Formulations screening

| Polymer | Solvent | Solvent % | [Polymer] | | Size (nm) | Pdl | Zpot (mV) | pH |
|---|---|---|---|---|---|---|---|---|
| CSHCl | H2O | | 1 | mg/mL | 168.3 | 0.184 | 39 | 4.49 |
| CSUltrapure | H2O | | 1 | mg/mL | 341 | 0.445 | 5.56 | — |
| AL-120 - 80% DDA | HCl 1N | 0.75 | 1 | mg/mL | 1.59E+04 | 0.195 | 66.2 | 3.11 |
| | | | 0.5 | mg/mL | 1.92E+04 | 1 | 53.3 | 3.25 |
| | | 0.92 | 1 | mg/mL | 994.9 | 1 | 64 | 2.5 |
| | Acetic Acid | 1 | 1 | mg/mL | 1.29E+04 | 0.049 | 72 | 2.73 |
| | | | 0.5 | mg/mL | 1.31E+04 | 0.345 | 69.8 | 2.83 |
| AL-476 - 90% DDA | HCl 1N | 0.75 | 1 | mg/mL | 4582 | 0.279 | 62.3 | 1.96 |
| | | | 0.5 | mg/mL | 1771* | 1 | 55.2 | 2.26 |
| | | 0.89 | 1 | mg/mL | 1.03E+04 | 1 | 64.5 | 2.35 |
| | Acetic Acid | 1 | 1 | mg/mL | 7308 | 0.51 | 72.2 | 2.81 |
| | | | 0.5 | mg/mL | 1.28E+04 | 0.458 | 64.1 | 2.84 |
| A-90101 - 90.1% DDA | HCl 1N | 0.5 | 1 | mg/mL | 2247 | 1 | 45.8 | 3.32 |
| | | | 0.5 | mg/mL | 2232 | 1 | 41.9 | 3.41 |
| | | 0.75 | 1 | mg/mL | 309.4 | 0.198 | 54.6 | 2.73 |
| | | | 0.5 | mg/mL | 1776 | 1 | 34.9 | 2.58 |
| | Acetic Acid | 1 | 1 | mg/mL | 1922 | 1 | 72.2 | 2.8 |
| | | | 0.5 | mg/mL | 3.46E+04 | 0.478 | 64.1 | 2.92 |
| CM95401132 - 98% DDA | HCl 1N | 0.5 | 1 | mg/mL | 771.3 | 1 | 5.01 | 3.37 |
| | | | 0.5 | mg/mL | 6.52E+04 | 1 | 19.3 | 3.55 |
| | | 0.61 | 1 | mg/mL | 4.25E+04 | 1 | 70.5 | 3.05 |
| | Acetic Acid | 1 | 1 | mg/mL | 3.46E+04 | 1 | 77.9 | 2.74 |
| | | | 0.5 | mg/mL | 7.34E+04 | 1 | 75.6 | 2.85 |

TABLE 3
Composition of the chitosan nanocapsules (CS nanocapsules)

| Product | %(w/w) |
|---|---|
| Miglyol 812N | 1.24 |
| Ethanol 100% | 7.49 |
| Lipoid S75 | 0.2 |
| CS HCl GMP | 0.02 |
| Water | 90.59 |

### Example 3. Comparison of In Vitro Toxicities with Other Formulations without Chitosan

In later phases of the project, three nanoformulations were studied, all with similar properties and quality attributes but using different polymers. The following figures show the qualitative composition of each of them (Table 4), as well as the values of their quality attributes (Table 5), all of which are very similar regardless of the polymer used.

TABLE 4
Qualitative compositions of the different formulations studied.

| Chitosan formulation | Polyarginine formulation | Protamine formulation |
|---|---|---|
| Miglyol 812N | Miglyol 812N | DL-α-tocopherol |
| Ethanol 100% | Ethanol 100% | Tween 80 |
| Lipoid S75 | Lipoid S75 | Sodium cholate |
| CS HCl | nBu-PArg | Ethanol 100% |
| MilliQ water | MilliQ water | Protamine YGK |
| | | MilliQ water |

TABLE 5
Values of the quality attributes of
the different formulations studied.

| | Size (nm) | Pdl | Zpot (mV) | pH |
|---|---|---|---|---|
| Chitosan | 250 ± 20 | 0.2 ± 0.05 | 25 ± 10 | 4.5 ± 0.3 |
| Polyarginine | 200 ± 20 | 0.1 ± 0.05 | 60 ± 5 | 5.5 ± 0.2 |
| Protamine | 200 ± 10 | 0.15 ± 0.05 | 20 ± 5 | 6.5 ± 0.2 |

The toxicities of the formulations were determined in RAW 264.7 cells (mouse macrophages), previously stimulated with LPS (lipopolysaccharide), which allows monocytes to differentiate into a proinflammatory state (as M1 macrophages). Different dilutions of the three formulations were studied over a 24-hour incubation period, allowing the generation of dose-dependent cell viability curves and their TC50 value, which corresponds to the concentration at which a formulation causes 50% cell death. Comparing the results, a clear difference in toxicity between them is observed. The chitosan prototype (hereinafter CS nanocapsules)—which comprises 0.225 mg/ml of chitosan—is clearly less toxic, not reaching 50% toxicity until a 1:4 dilution (1 part of the initial formulation diluted in 3 parts of cell medium). Protamine and polyarginine have more similar toxicities, although it is true that polyarginine is somewhat more toxic, especially because from 1:32 cell viability declines much faster (FIG. 2).

### Example 4. Comparison of In Vitro Efficacy with Other Chitosan-Free Formulations

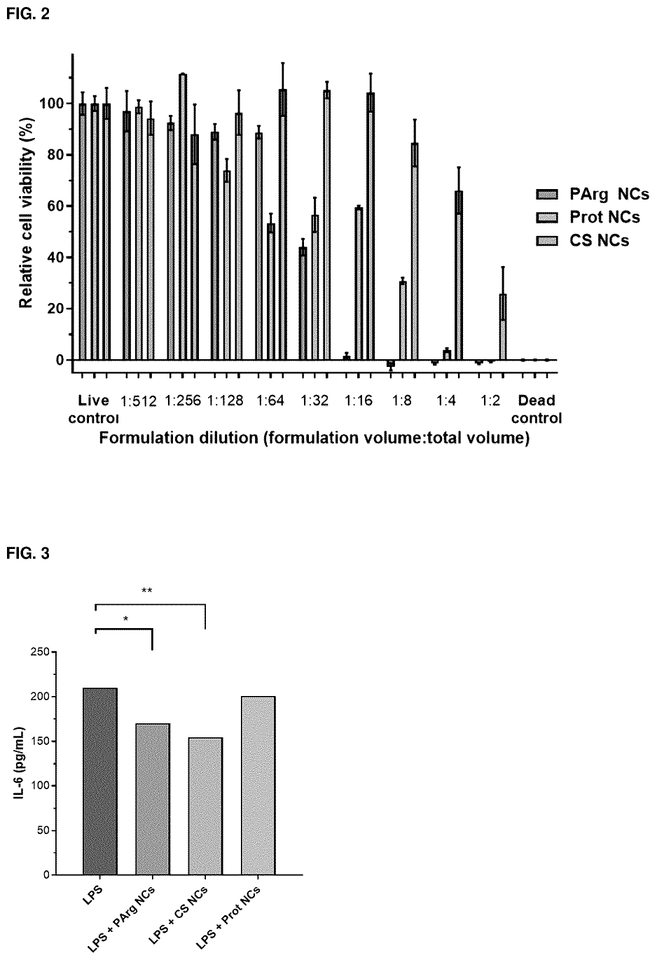

After completing the toxicity study, the efficacy of LPS treatment in reducing inflammation in macrophages was studied. To this end, the cytokine levels present in the supernatants from previous toxicity experiments were analyzed using ELISA. Specifically, the study of the levels of the proinflammatory cytokine IL-6 showed a significant reduction (p=0.030), proving the anti-inflammatory capacity of the chitosan formulation. It was the only one that managed to reduce these values with a significance of ** (FIG. 3).

### Example 5. Animal Studies

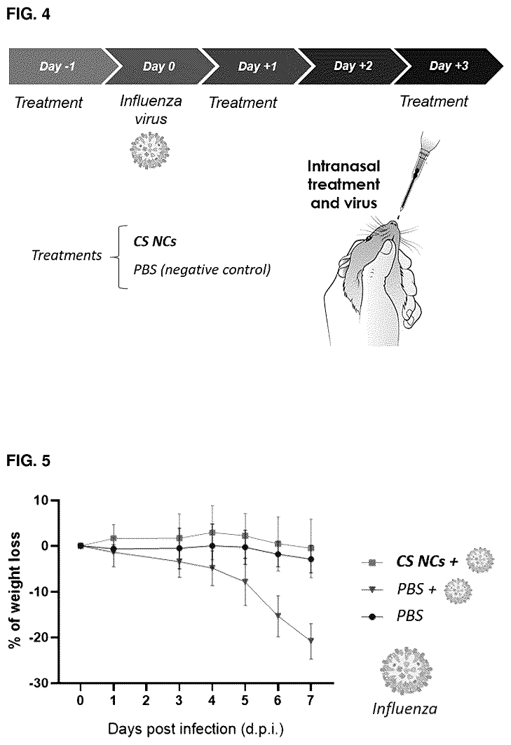

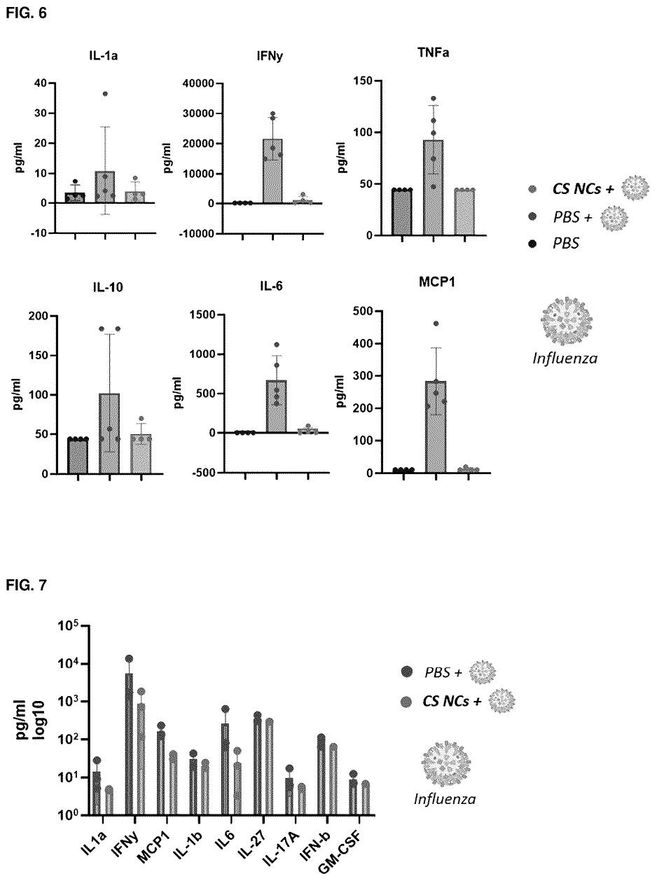

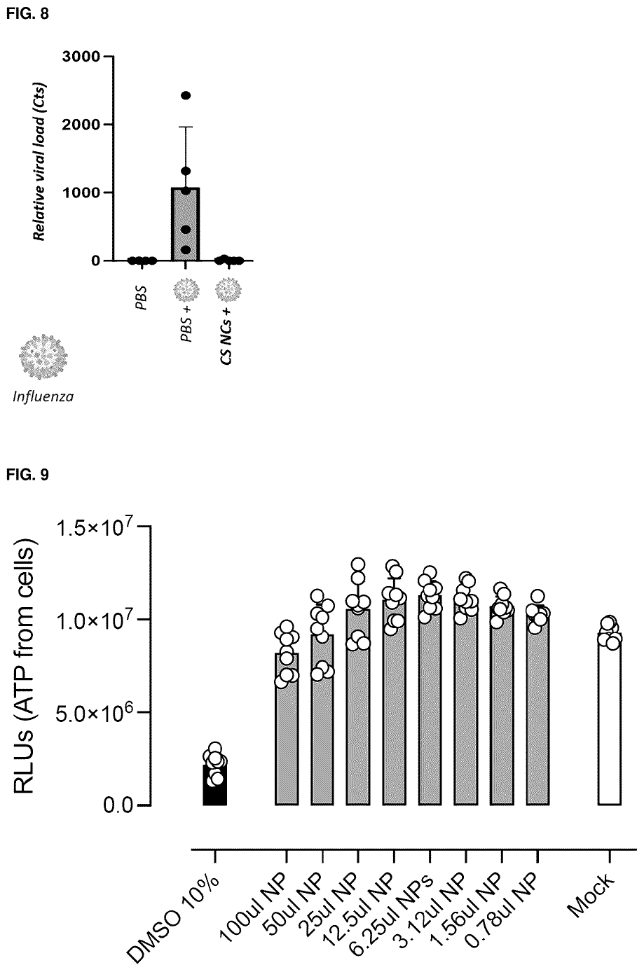

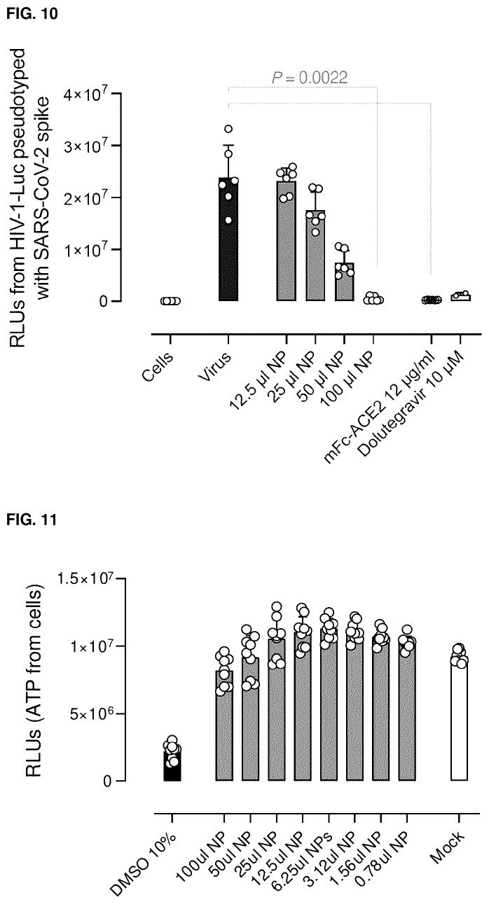

The results of the influenza efficacy experiments in animals, with intranasal treatments of 5 μL per nostril of the nanoformulation described in Table 3 on days −1, +1 and +3 (0 being the day of infection with H1 N1 PR8 influenza) (FIG. 4), were performed in duplicate to ensure reproducibility and showed an unprecedented capacity to prevent influenza contagion. Thus, 100% of the animals treated with our CS nanocapsules formulation did not develop infection after having been in contact with the virus. This fact was fully validated by analyzing multiple parameters such as their weight (FIG. 5), levels of inflammation in the lung (FIG. 6), levels of inflammation in the blood (FIG. 7) and viral load in the lung (FIG. 8), all of which corresponded to values typically found in non-infected animals.

### Example 6. In Vitro Toxicity of the Chitosan Nanocapsules in 293T Cells

A cellular cytotoxicity assessment by cell metabolic activity was performed in 293-ACE2 cells in order to determine the maximum amount of nanoformulation that can be employed in viral entry studies. The experiment was performed 3 times in triplicates for 8 dilutions of the nanocapsules (NPs). 293-ACE2 cells were incubated with different dilutions of the formulation in cell media for 48 h. Then, cells were washed with PBS and ATP-dependent light emission was measured as an indication of cell viability.

The results showed a dose-dependent toxicity. Moreover, it should be noted that even the highest volume of formulation studied (100 μL of formulation, which is the maximum for the experimental design of viral entry studies, and which is equivalent to a 1:2 dilution of the formulation shown in Table 3), is not toxic for cells, as it does not reduce cell viability more than 10-15%.

### Example 7. Viral Entry Study to Search for Pan-Coronavirus Entry Inhibition of NPs in ACE-2 Expressing 293T Cells

In this study, the inhibition of SARS-CoV-2 pseudovirus fusion was studied in 293-ACE2 cells using the nanoformulation (NPs) described in Table 3. The assay is based on a single-cycle, envelope-deficient HIV-1 reporter virus expressing the luciferase gene. This pseudovirus is pseudotyped with the SARS-CoV-2 spike protein, which facilitates viral fusion with 293-ACE2 cells. The pseudovirus was combined with the formulation at different concentrations and the mixture was incubated with the cells for 48 h to detect viral entry. Luciferase expression served as a readout for successful fusion events.

The results showed a clear dose-dependent inhibitory effect of the tested NPs on viral fusion. The formulation effectively reduces viral fusion at concentrations that do not exhibit cytotoxicity in 293-ACE2 cells (as demonstrated in Example 6 and FIG. 9). Moreover, we can see that the formulation at higher concentrations is able to inhibit fusion as effectively as a recombinant murine Fc-ACE2 protein or an HIV-1 integration inhibitor such as dolutegravir.

### Example 8. Additional Experiments Demonstrating the Prophylactic Effect of the Chitosan Nanocapsules in SARS-Cov2, Respiratory Syncytial Virus (RSV) and HIV-1

### Example 8.1. Materials and Methods

### Reagents

The SV RESPVIR B-I formulation corresponds to the formulation of Table 3, and was prepared as indicated in Example 1.1.
Soluble ACE2-mFc (12.5 ug/mL) was used as a control to inhibit the cellular entry via the SARS-CoV-2 receptor ACE-2.
The integrase inhibitor Dolutegravir (10 uM) was used as a control to inhibit HIV integration.
Remdesivir (25-50 μM) was used as a golden standard antiviral control to inhibit the RNA-dependent RNA polymerase (RdRp) in both SARS-CoV-2 and respiratory syncytial virus (RSV) assays.

### Cell Cultures

HEK-293T overexpressing the human receptor ACE2 (HEK293T-ACE2 cells) were acquired from BEI Resource (Catalog No. NR-52511) and maintained in DMEM (Invitrogen) with 10% fetal bovine serum, 100 IU/mL penicillin and 100 μg/mL streptomycin (all from Invitrogen).
The African Green Monkey kidney epithelial cell line Vero E6 (Vero E6) was acquired from ATCC (Catalog No. CRL-1586). These cells were maintained with D10 medium composed of DMEM with 10% fetal bovine serum (FBS), 100 IU/mL penicillin, and 100 μg/mL streptomycin (all from Invitrogen). Of note, these cells lack the capacity to produce interferons and are therefore highly susceptible to viral-induced cytopathic effects upon infection. Thus, this is one of the most demanding in vitro models to observe antiviral activity, as cells are not aided by the release of interferons in the culture.
U87 expressing the human receptor CD4 and co-receptor CXCR4 (U87 CD4+ CXCR4+ cells) were acquired from NIH AIDS Reagent Program (Catalog No. 4036) and maintained in DMEM with 10% fetal bovine serum, 100 IU/mL penicillin and 100 μg/mL streptomycin, supplemented with Geneticin (0.5 mg/ml) and Puromycin (1 μg/ml), all from Invitrogen.

### Viral Stocks

HIV-1 reporter pseudoviruses expressing SARS-CoV-2 Spike protein and luciferase (named SARS-CoV-2 pseudoviruses in the following lines) were generated using plasmids pNL4-3.Luc.R-.E- (NIH AIDS repository) and a modified SARS-CoV-2.SctΔ19 Spike from Beta variant as described by Trinité et al. 2021(1).
SARS-CoV-2 viral stocks were produced in Vero E6 cells with D10 medium. Genomic sequences from SARS-CoV-2 variants D614G (ID EPI_ISL_510689), JN1 (EPI_ISL_18838623), and KP2 (EPI_ISL_19264939) were then deposited at GISAID (http://gisaid.org). Viral stocks were titrated by the “Muench & Reed” method as described in Rodon et al. 2021(2).
HIV-1 virus was generated using HIV-1 recombinant virus containing GagPol-coding region of a WT HIV-1 HXB2 strain (http://www.hiv.lanl.gov) (Genbank accession number: K03455) and plasmid pNL4-3 Δ gag-pol produced in-house. In MT-4 cells, the GagPol(HXB2) PCR product was recombined with a GagPOL-deleted HIV-1 infectious clone (pNL4-3, from NIH) that had been previously linearized with BstE II. Virus titration was performed in MT-4 cells, and values were expressed as tissue culture dose for 50% infectivity (TCID50 of 88246.8 U/ml).
RSV viral stocks (RSV-GFP5; #R125; ViraTree) were grown in Vero E6 cells. Cell associated GFP signal was measured by an ELISA (Abcam): 2295 ng/ml. Viral stocks were titrated by the “Muench & Reed” method as described in Rodon et al. 2021(2).

### Cytotoxicity Assay

To evaluate drug-associated cytotoxic effects, 60.000 cells per well were seeded in 96-well plates and exposed to increasing concentrations of SV RESPVIR B-I nanoparticles, non-formulated components and positive control for virus inhibition (Remdesivir or Dolutegravir). No virus was added to these wells. Each condition was evaluated in duplicate in 3 independent experiments (n=3). Cells were lysed with CellTiter-Glo® Luminescence Cell Viability Assay (Promega), which reacts with ATP from viable cells. Luminescence was measured with a Luminoskan luminometer (Thermo Scientific) with an integration time of 2 sec. The RLUs were normalized, and the IC50 was calculated by plotting and fitting the log 10 of each compound concentration vs. cytotoxicity to a 4-parameter equation. All analyses and figures were generated with the GraphPad Prism v10.4 Software.

### Pseudovirus Assay

To assess viral fusion, HEK-293T-ACE2 cells were exposed to SARS-CoV-2 pseudoviruses in the presence of increasing concentrations of nanoparticles, components and fusion or antiviral controls, lysed 48 h post-inoculation with the Bright-Glo™ Luciferase Assay System (Promega) to detect viral fusion via luciferase quantification. To avoid any possible interference of luminescent detection from nanoparticles, cells were washed 3 times with PBS before adding each of the corresponding luminescent reagents. Luminescence was measured with an EnSight Multimode Plate Reader (Perkin Elmer) with an integration time of 2 sec. All analyses and figures were generated with the GraphPad Prism v10.0b Software.

### Antiviral Assays

To evaluate viral-associated cytopathic effect cells were infected with SARS-CoV-2, HIV-1 or RSV and exposed to increasing non-cytotoxic concentrations of SV RESPVIR B-I formulation, non-formulated components and positive control for virus inhibition (Remdesivir or Dolutegravir). We used a constant viral amount for each virus selected to reach 50% of viral-induced cytopathic effect. Cells were lysed and measured as indicated to detect cytotoxicity. All analyses and figures were generated with the GraphPad Prism v10.0b Software.

### Example 8.2. Results

### Example 8.2.1. Study of the Capacity of SV RESPVIR B-I Formulation to Inhibit SARS-CoV-2 Fusion in ACE2 HEK-293T Cells

The specific aims were the following:
To assess cytotoxicity induced by SV RESPVIR B-I nanoparticles on human embryonic kidney cells expressing ACE2 (HEK293T-ACE2) in the absence of virus.
To assess the inhibition of SARS-CoV-2 pseudoviral membrane fusion on HEK293T-ACE2 cells after the addition of SV RESPVIR B-I nanoparticles (100 ul, 50 ul, 25 ul, 12.5 ul, 6.25 ul, 3.12 ul, 1.56 ul, 0.78 ul).

The nanoparticles showed an antiviral effect at the highest concentration tested abrogating pseudoviral entry into HEK293T-ACE2 cells at non-cytotoxic concentrations.

An in vitro cellular model was used to test the cytotoxic and the antiviral effects of the nanoparticles in HEK293T-ACE2 cells. This system measures luciferase produced upon SARS-CoV-2 pseudoviral entry into a susceptible cellular line. As a control, cellular viability at increasing concentrations of SV RESPVIR B-I formulation was assessed in the absence of SARS-CoV-2 pseudoviruses, in this case using a system that uses ATP released by viable cells as cofactor for the reaction that produces luciferase and can be measured in relative light units (RLUs). None of the tested concentrations showed nanoparticle-associated cytotoxicity (FIG. 11).

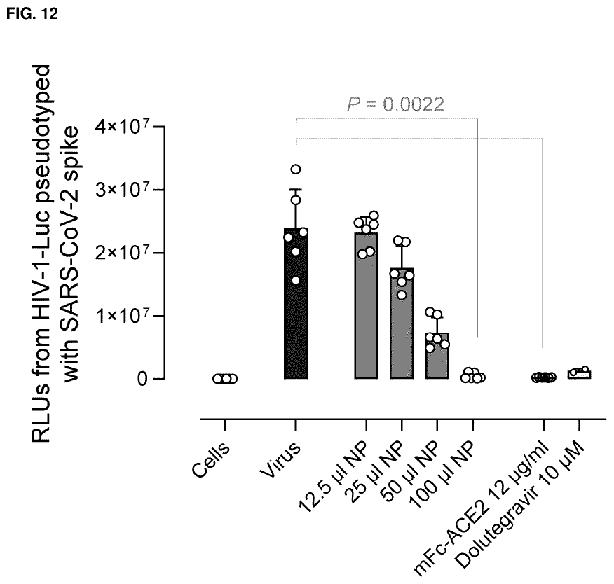

To assess the antiviral effect of the nanoparticles, we performed 3 independent experiments with 2 replicates. Nanoparticles were added to the cells in a 1:2 serial dilutions before adding a constant concentration of SARS-CoV-2 pseudovirus. Cells were left incubating for 48 h before assessing luciferase signal. We saw a complete inhibition of the entry of the pseudoviruses with the highest concentration of nanoparticles that lost efficacy in a dose dependent manner (FIG. 12). As a positive control for viral fusion inhibition, soluble ACE2-mFc completely abrogated entry (FIG. 12). In addition, the HIV-1 integrase inhibitor Dolutegravir effectively inhibited HIV-1 driven luciferase expression (FIG. 12).

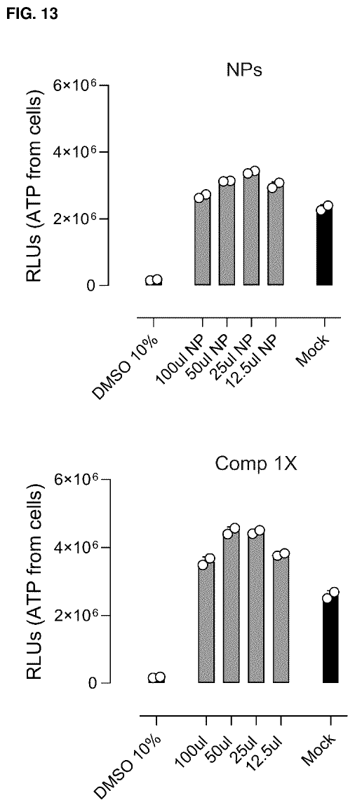

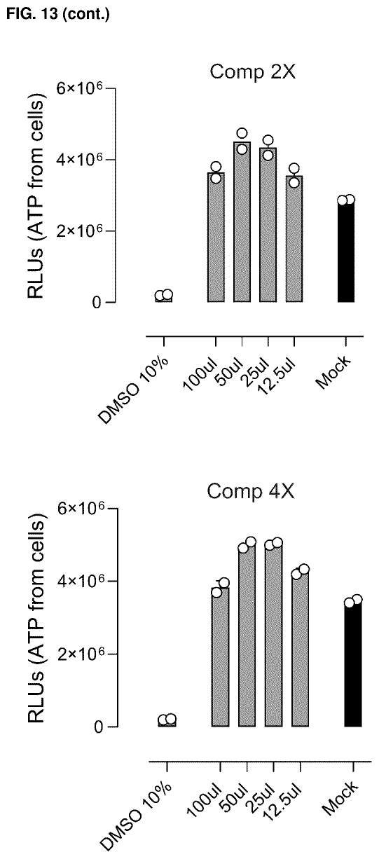

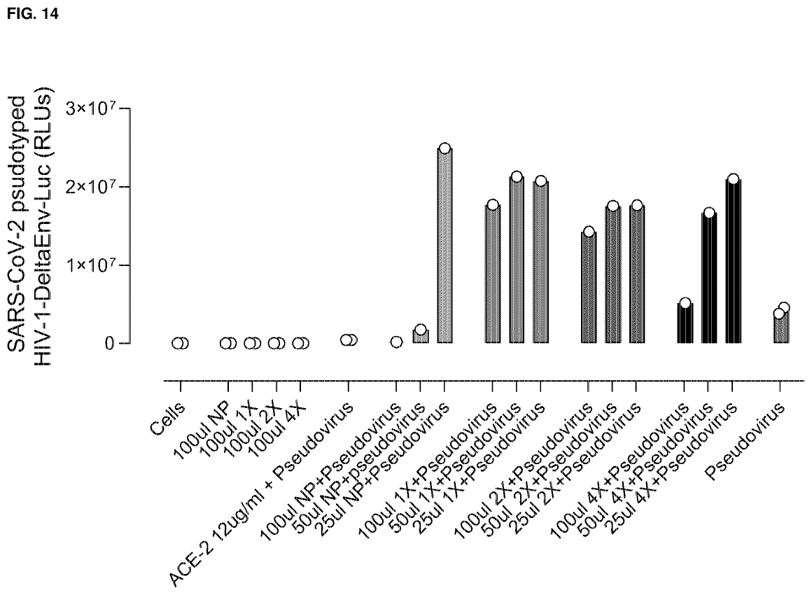

Finally, we also tested if increasing concentrations of the components used to formulate the nanoparticles could also display antiviral activities in one experiment performed in 2 replicates. In the absence of cytotoxic effects of the non-formulated compounds (FIG. 13) we could confirm that there was no antiviral efficacy detected (FIG. 14).

### Example 8.2.2. Study of the Capacity of SV RESPVIR B-I Formulation to Inhibit SARS-CoV-2 Replication in Vero E6 Cells

The specific aims were the following:
To assess the cytotoxic effect associated with SV RESPVIR B-I nanoparticles at different concentrations (4.675-37.4 μg/μl) in the absence of virus using the Vero E6 cellular model.
To assess the inhibition of viral replication by measuring the decrease in viral-induced cytopathic effect (CPE) associated with SARS-CoV-2 variants D614G, JN1, and KP2 after adding SV RESPVIR B-I nanoparticles at different concentrations (4.675-37.4 μg/μl) using the Vero E6 cellular model.

Nanoparticles had the capacity to inhibit SARS-CoV-2 replication in all variants tested in high and low infectivity conditions. Antiviral activity was dose-dependent, reaching an IC50 of 16.65 μg/μl for D614G, 15.72 μg/μl for JN1, and 29.23 μg/μl for KP2 at higher infectivity assays. No nanoparticle-associated cytotoxic effect was observed at any of the concentrations tested. Components used to formulate nanoparticles (1×) showed no comparable inhibitory capacity at the same concentration used for nanoparticles.

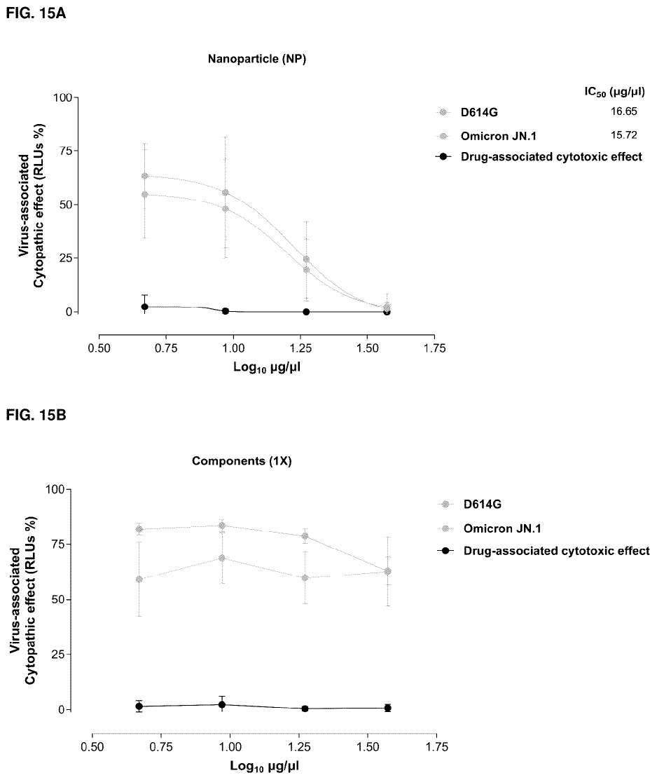

We first measured the inhibitory capacity of a serial dilution (1:2) of nanoparticles in an infectious assay set up to reach a 50% viral-induced cytopathic effect (FIG. 15A). D614G original variant and JN1 omicron variant show a highly divergent sequence that evolved throughout the pandemic. Both variants were equally sensitive to the inhibition of SV RESPVIR B-I nanoparticles (color lines), as well as control remdesivir (25 μM), at doses that were not cytotoxic (dark lines). In contrast, non-formulated components (FIG. 15B) showed no comparable inhibitory effect (color lines) at the same concentrations tested. None of the doses were cytotoxic (dark line).

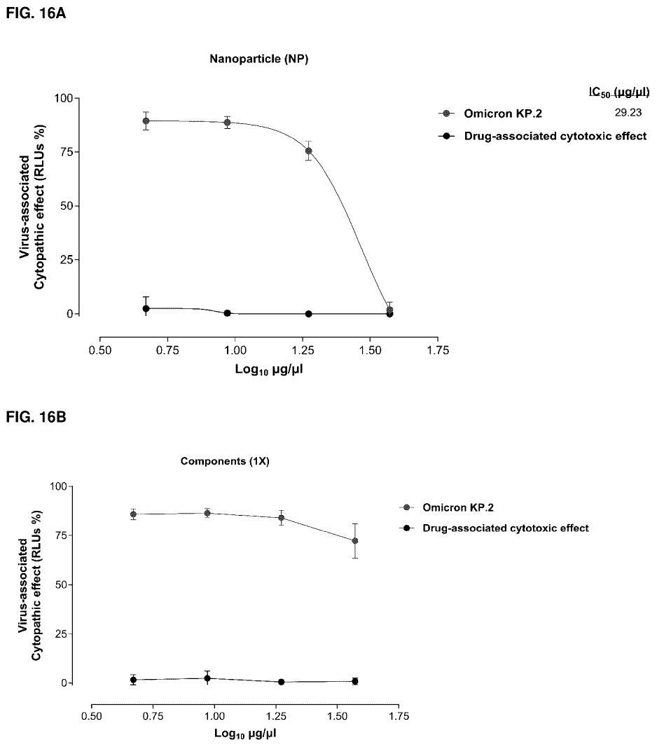

Similar results were obtained for KP2 variant in an experiment set up to reach a 75% viral-induced cytopathic effect (FIG. 16A). Cells were equally inhibited by nanoparticles (color line) and remdesivir (25 μM) at doses that were not cytotoxic (dark lines), and this was not the case for non-formulated components (FIG. 16B).

Nanoparticles (A) and non-formulated components (B) were tested in concentrations ranging from 4.675-34.40 μg/μl on VERO E6 cells infected with a low MOI of SARS-CoV-2 virus Omicron KP2. Data shows normalized (%) luciferase Relative Light Units (RLUs). IC50 is indicated.

### Example 8.2.2. Study of the Capacity of SV RESPVIR B-I Formulation to Inhibit HIV-1 Replications in U87 CD4+ CXCR4+ Cells

The specific aims were the following:
To assess cytotoxicity induced by SV RESPVIR B-I nanoparticles on human glioblastoma astrocytoma cell line expressing CD4 and CXCR4 that is susceptible to HIV-1 infection (U87 CD4+ CXCR4+) in the absence of HIV-1.
To assess the inhibition of viral replication by measuring the decreased in viral-induced cytopathic effect associated with HIV-1 on U87 CD4+ CXCR4+ cells after the addition of SV RESPVIR B-I nanoparticles at different concentrations.

Nanoparticles showed an antiviral effect at the highest non-cytotoxic concentrations tested abrogating HIV-1 entry into U87 CD4+ CXCR4+ cells.

An in vitro cellular model was used to test the cytotoxic and the antiviral effects of the nanoparticles of the SV RESPVIR B-I formulation in U87 CD4+ CXCR4+ cells infected with HIV-1.

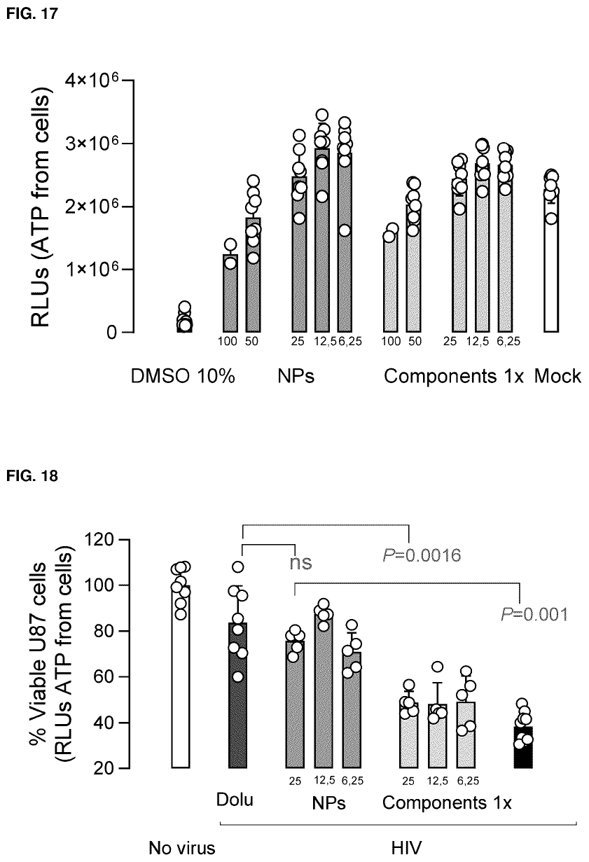

As a control for nanoparticle-induced cytotoxicity, cellular viability at increasing concentrations of the nanoparticles and in the absence of HIV-1 was assessed. We employed a system that uses ATP released by viable cells as cofactor for the reaction that produces luciferase and can be measured in relative light units with a luminometer (RLUs). The nanoparticles and components were assessed in 3 independent experiments with 3 replicates per cytotoxicity experiment 96 h post nanoparticle addition, to mimic the HIV-1 assay. Only the two highest concentrations tested showed nanoparticle/components-associated cytotoxicity while the rest of the concentration tested were not toxic (FIG. 17).

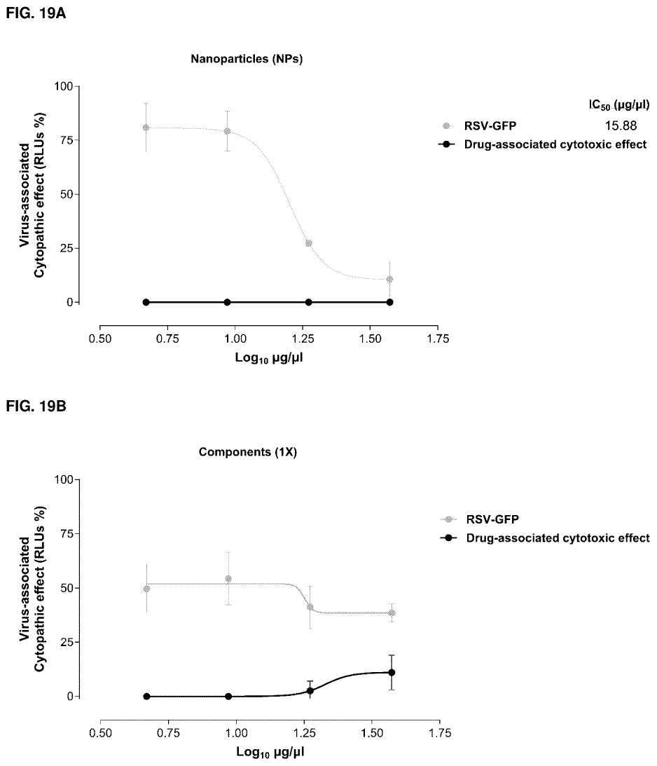

To assess the antiviral effect of the nanoparticles, we performed 3 independent experiments with 2 replicates using non-cytotoxic concentrations bellow 50 ul. Nanoparticles and components were added to the cells in a 1:2 serial dilutions starting with the highest non-cytotoxic concentration. Then, a constant concentration of HIV-1 was added. Cells were left incubating for 96 h before measuring the viral-induced cytopathic effect. As observed in FIG. 2, using the nanoparticles of the SV RESPVIR B-I formulation, we found an inhibition of viral-induced cytopathic effect comparable to our positive control (dolutegravir 10 uM), and the effect was double that of the components alone or the virus without any treatment (FIG. 18).

### Example 8.2.3. Study of the Capacity of SV RESPVIR B-I Formulation to Inhibit RSV Replication in Vero E6 Cells

The specific aims were the following:
To assess the cytotoxic effect associated with SV RESPVIR B-I nanoparticles at different concentrations (4.7-37.4 μg/μl) in the absence of virus using the Vero E6 cellular model.
To assess the inhibition of viral replication by measuring the decrease in viral-induced cytopathic effect (CPE) associated with RSV after adding SV RESPVIR B-I nanoparticles at different concentrations (4.7-37.4 μg/μl) using the Vero E6 cellular model.

Nanoparticles had the capacity to inhibit RSV replication. Antiviral activity was dose-dependent (4.7-37.4 μg/μl), reaching an IC50 of 15.9 μg/μl. No nanoparticle-associated cytotoxic effect was observed at any of the concentrations tested. Components used to formulate nanoparticles (1×) showed no comparable inhibitory capacity. No cytotoxicity was identified except for the highest dose.

We measured the inhibitory capacity of a serial dilution (1:2) of nanoparticles in an RSV infectious assay set up to reach a 50% viral-induced cytopathic effect (FIG. 19A). RSV was sensitive to the inhibition of nanoparticles (blue lines), at doses that were not cytotoxic (dark lines). In contrast, non-formulated components (FIG. 19B) showed no comparable inhibitory effect (blue lines) at the same concentrations tested. No cytotoxicity (dark line) was identified except for the highest dose in the non-formulated components (10.95%).

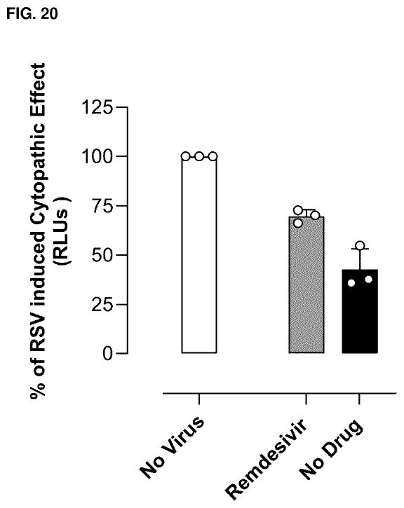

Of note, antiviral activity was also detected using Remdesivir at 50 uM, although it did not reach al full inhibitory effect (FIG. 20).

### REFERENCES

(1) Trinité, B., Tarrés-Freixas, F., Rodon, J., Pradenas, E., Urrea, V., Marfil, S., Rodríguez de la Concepcién, M. L., Ávila-Nieto, C., Aguilar-Gurrieri, C., Barajas, A., Ortiz, R., Paredes, R., Mateu, L., Valencia, A., Guallar, V., Ruiz, L., Grau, E., Massanella, M., Puig, J., Chamorro, A., Izquierdo-Useros, N., Segalés, J., Clotet, B., Carrillo, J., Vergara-Alert, J., Blanco, J., 2021. SARS-CoV-2 infection elicits a rapid neutralizing antibody response that correlates with disease severity. Sci Rep 11, 2608. https://doi.org/10.1038/s41598-021-81862-9.
(2) Rodon, J., Muñoz-Basagoiti, J., Perez-Zsolt, D., Noguera-Julian, M., Paredes, R., Mateu, L., Quiñones, C., Perez, C., Erkizia, I., Blanco, I., Valencia, A., Guallar, V., Carrillo, J., Blanco, J., Segalés, J., Clotet, B., Vergara-Alert, J. and Izquierdo-Useros, N., 2021. Identification of Plitidepsin as Potent Inhibitor of SARS-CoV-2-Induced Cytopathic Effect After a Drug Repurposing Screen. Frontiers in Pharmacology, 12. DOI: 10.3389/fphar.2021.646676.

### Example 9—Evaluation of SV RESPVIR B-I in a Physiological Human Nasal Epithelium Model and In Vivo Biodistribution Study

### Objective

The objective of this example was to evaluate the antiviral performance, epithelial residence time, and safety of the SV RESPVIR B-I, the nanocapsules of the present disclosure, formulation in a physiological model of the human nasal epithelium and to characterize its in vivo biodistribution following intranasal administration. In particular, the antiviral activity of the formulation against SARS-CoV-2 was assessed using the MucilAir human nasal epithelium model, a physiologically relevant in vitro system that closely reproduces the structure and function of the human nasal mucosa. In parallel, the biodistribution and tissue localization of SV RESPVIR B-I were investigated in a murine model using fluorescence-based imaging techniques.

### Materials and Methods

### Human Nasal Epithelium Model

The MucilAir human upper airway epithelium model (Epithelix; EP01 MD), cultured at the air-liquid interface, was used. This model is composed of basal progenitor cells, mucus-producing goblet cells, and ciliated epithelial cells, and displays dense ciliation, mucus secretion, functional mucociliary clearance, intact tight junctions, ion channels, and native cell surface receptors, including ACE2 and TMPRSS2. The model also retains downstream innate immune response capabilities. Cultures were maintained using a commercial medium (EP04MM, Epithelix).

### Cytotoxicity Assessment

To assess potential cytotoxic effects, MucilAir inserts containing approximately 500,000 cells were treated with increasing concentrations of SV RESPVIR B-I nanoparticles or corresponding non-formulated components (9.35-74.8 μg/μl, 2× formulation) for 72 hours. Untreated cells served as negative controls. Cell viability was quantified using the CellTiter-Glo® luminescence assay, which measures ATP as an indicator of metabolically active cells. Luminescence values were normalized to untreated controls.

### Type I Interferon Gene Expression Analysis

To evaluate whether SV RESPVIR B-I modulates innate immune signaling, MucilAir tissues were treated with nanoparticles or components (9.35-74.8 μg/μl) for 72 hours. Poly I:C (12.5 ng/μl) was used as a positive control for interferon pathway activation. Total RNA was extracted, retrotranscribed, and IFNα1 and IFNβ1 gene expression was measured by RT-PCR. Expression levels were normalized to untreated controls.

### Antiviral Efficacy and Residence Time Analysis

To assess antiviral activity and persistence on the epithelial surface, MucilAir tissues were treated with SV RESPVIR B-I nanoparticles or components at concentrations of 18.70 μg/μl and 74.8 μg/μl and infected with a nanoluciferase-expressing SARS-CoV-2 reporter virus (nLuc-SCV2, MOI 0.04). For residence-time analysis, tissues were infected at 0, 4, 8, 24, or 48 hours after nanoparticle application. Viral infectivity was quantified 72 hours post-infection using the NANO-GLO® luminescence assay. Results were normalized to infected untreated controls.

### In Vivo Biodistribution Study

For biodistribution analysis, SV RESPVIR B-I nanoparticles were labeled with rhodamine as a fluorescent tracer. Mice were intranasally administered 40 μl of the formulation (20 μl per nostril). Whole-body fluorescence imaging was performed using an in vivo imaging system at defined time points following administration. After the final imaging session, animals were sacrificed and ex vivo tissues, including nasal epithelium, lungs, liver, kidneys, and plasma, were collected. Rhodamine fluorescence was extracted from tissue homogenates and quantified to determine formulation localization.

### Results

### Cytotoxicity

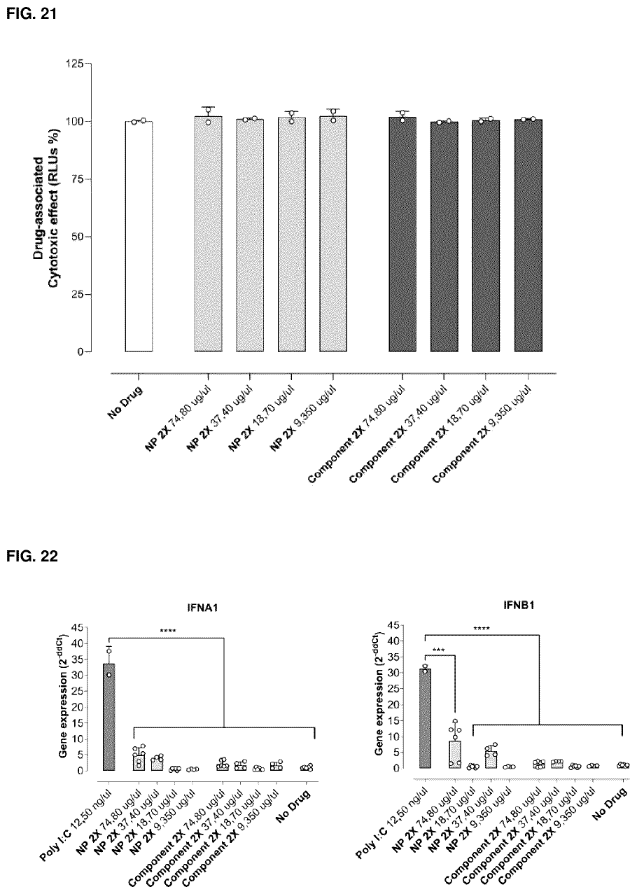

No cytotoxic effect associated with SV RESPVIR B-I nanoparticles or non-formulated components was detected in the MucilAir model at any of the tested concentrations (9.35-74.8 μg/μl). Cell viability remained comparable to untreated controls after 72 hours of exposure (FIG. 21).

### Interferon Pathway Analysis

SV RESPVIR B-I nanoparticles did not induce type I interferon gene expression in the MucilAir model. IFNα1 and IFNβ1 mRNA levels in nanoparticle-treated tissues remained similar to untreated controls, whereas robust induction of both genes was observed with the Poly I:C positive control. Only a slight increase in IFNβ1 expression was observed at the highest nanoparticle concentration, which was substantially lower than that induced by Poly I:C (FIG. 22).

### Antiviral Activity Against SARS-CoV-2

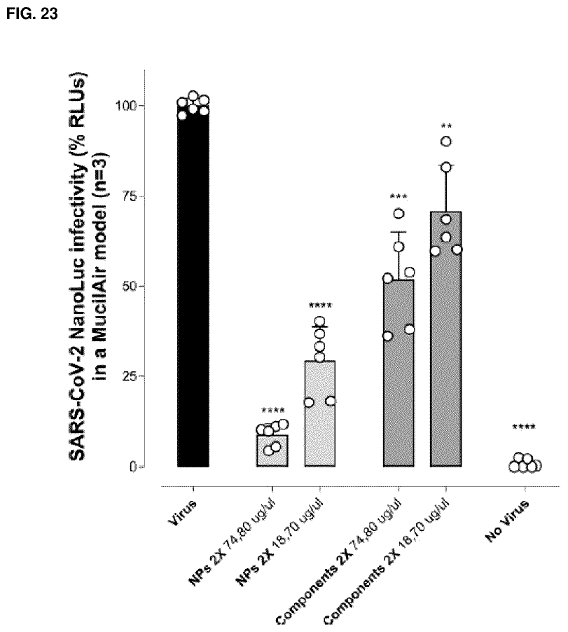

SV RESPVIR B-I nanoparticles inhibited SARS-CoV-2 infection in the MucilAir model in a dose-dependent manner. Viral infectivity was reduced by approximately 70% to 91% at concentrations of 18.70 μg/μl and 74.8 μg/μl, respectively. In contrast, non-formulated components exhibited markedly lower antiviral activity under the same conditions (FIG. 23).

### Residence Time and Sustained Antiviral Efficacy

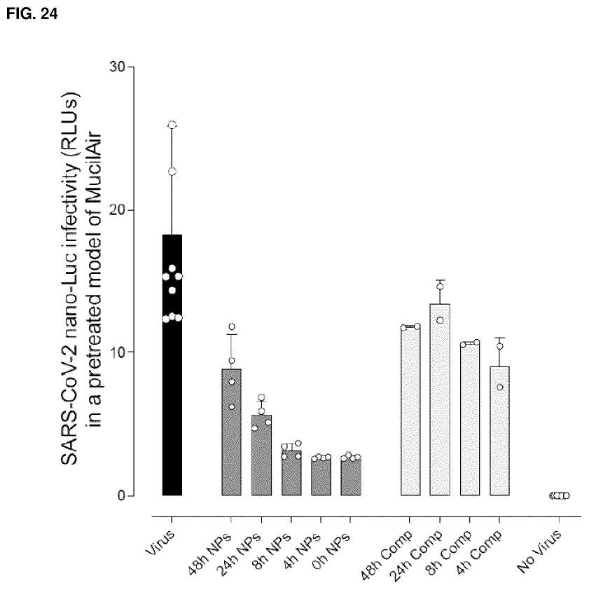

The nanoparticle formulation demonstrated prolonged residence and sustained antiviral activity on the nasal epithelium. Full antiviral efficacy was maintained for up to 8 hours post-application, and approximately 70% of antiviral activity was retained at 24 hours. These results indicate that SV RESPVIR B-I remains associated with the epithelial surface for extended periods while maintaining functional antiviral activity (FIG. 24).

### In Vivo Biodistribution

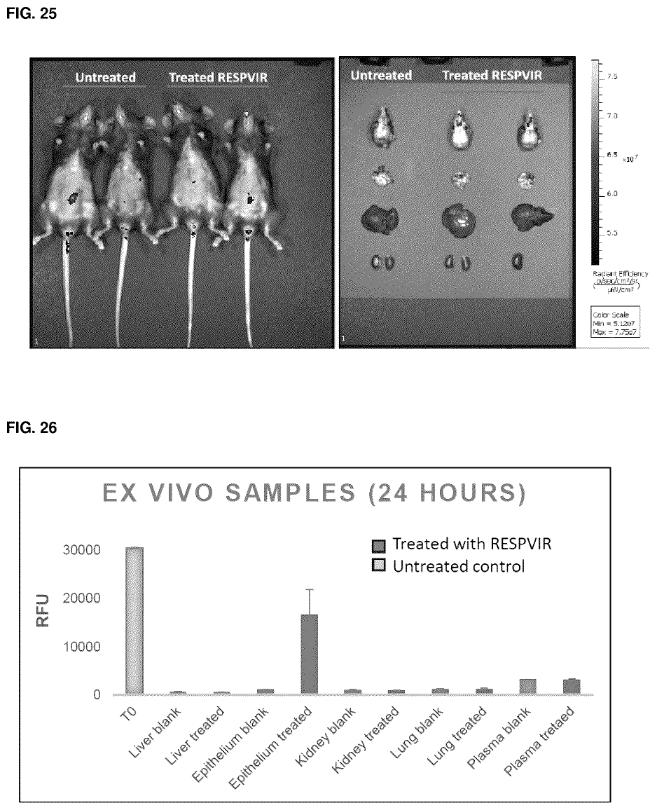

In vivo fluorescence imaging revealed that rhodamine-labelled SV RESPVIR B-I was predominantly localized in the nasal region following intranasal administration. Ex vivo imaging and quantitative fluorescence analysis confirmed high fluorescence levels in the nasal epithelium and negligible signals in lungs, liver, kidneys, and plasma. These findings indicate that the formulation remains localized at the site of application with no detectable systemic distribution (FIGS. 25 and 26).

### CONCLUSION

This example demonstrates that SV RESPVIR B-I is a non-toxic intranasal formulation that exhibits sustained antiviral activity against SARS-CoV-2 in a physiologically relevant human nasal epithelium model, without inducing type I interferon responses. The formulation shows prolonged residence on the nasal epithelium and remains confined to the nasal cavity following intranasal administration in vivo, supporting a localized mode of action consistent with a mechanical barrier and viral entry-inhibiting effect.

1. A method for prophylactic treatment of an infection caused by a respiratory virus in a subject in need thereof, comprising administering a nanocapsule system to the subject intranasally, wherein the nanocapsule system comprises:
a) a surface layer consisting of chitosan,
b) an oily core, and
c) a surfactant.

2. The method according to claim 1, wherein the respiratory virus is selected from the group consisting of influenza virus, respiratory syncytial virus (RSV), human rhinovirus (HRV), human parainfluenza virus (HPIV), adenovirus, human metapneumovirus (hMPV), severe acute respiratory syndrome coronavirus 2 (SARS-CoV-2), severe acute respiratory syndrome coronavirus (SARS-CoV), Middle East respiratory syndrome coronavirus (MERS-CoV), endemic human coronaviruses, bocavirus, and enteroviruses.

3. The method according to claim 2, wherein the respiratory virus is the influenza virus.

4. The method according to claim 2, wherein the respiratory virus is the severe acute respiratory syndrome coronavirus 2 (SARS-CoV-2).

5. The method according to claim 1, wherein the nanocapsule system consists of:
a) a surface layer consisting of chitosan,
b) an oily core, and
c) a surfactant.

6. The method according to claim 5, wherein the respiratory virus is the influenza virus.

7. The method according to claim 5, wherein the respiratory virus is the respiratory virus is the severe acute respiratory syndrome coronavirus 2 (SARS-CoV-2).

8. The method according to claim 5, wherein the oily core consists of an oil selected from the group consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof.

9. The method according to claim 8, wherein the oil is a medium-chain triglyceride having at least 90% w/w of triglycerides with three fatty acids having an aliphatic tail of 6 to 12 carbon atoms.

10. The method according to claim 9, wherein the oil is a medium-chain triglyceride having at least 90% w/w of a mixture of caprylic (C8) and capric (C10) triglycerides.

11. The method according to claim 1, wherein the surfactant is an oil-soluble surfactant.

12. The method according to claim 11, the oil-soluble surfactant is a phospholipid-based compound selected from the group consisting of lecithin, phosphatidylglycerol, phosphatidylserine, phosphatidylinositol, diphosphatidylglycerol, phosphatidic acid, phosphatidylcholine and phosphatidylethanolamine.

13. The method according to claim 12, wherein the phospholipid-based compound is selected from the group consisting of soy phospholipid with 70% phosphatidylcholine, hydrogenated lecithin, phosphatidylcholine, lecithin fraction with phosphatidylcholine content≥45%, phosphatidylcholine content≥94%, phospholipids with phosphatidylcholine content≥70%, phosphatidylcholine content≥90%, lecithin fraction with phosphatidylcholine content≥20%, phosphatidylcholine content≥94%, hydrogenated phospholipids with phosphatidylcholine content≥70%, hydrogenated phosphatidylcholine content≥90%, hydrogenated phospholipids with phosphatidylcholine content≥62%, hydrogenated phosphatidylcholine content≥90%, and hydrogenated phosphatidylcholine content≥90%.

14. The method according to claim 6, wherein the respiratory virus is the influenza virus and the nanocapsule system consists of:
a) a surface layer consisting of chitosan;
b) an oily core consisting of an oil selected from the group consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof; and
c) a surfactant, wherein the surfactant is an oil-soluble surfactant.

15. The method according to claim 7, wherein the respiratory virus is the severe acute respiratory syndrome coronavirus 2 (SARS-CoV-2); and the nanocapsule system consists of:
a) a surface layer consisting of chitosan;
b) an oily core consisting of an oil selected from the group consisting of mineral oil, squalene oil, flavor oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, neopentyl glycol dicarpate ketals, decyl oleate, C12-C1S alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, rapeseed oil, chili oil, coconut oil, fractionated coconut oil, corn oil, cottonseed oil, flax oil, oil of grape seed, mustard oil, olive oil, palm oil, fractionated palm oil, peanut oil, oil castor oil, pine seed oil, poppy seed oil, pumpkin seed oil, rice bran oil, safflower, tea oil, truffle oil, vegetable oil, apricot oil, jojoba oil, macadamia oil, wheat germ oil, almond oil, soybean oil, sesame oil, hazelnut oil, sunflower oil, hemp oil, bois oil, Kukui nut oil, avocado oil, walnut oil, fish oil, berry oil, allspice oil, juniper oil, seed oil, almond seed oil, anise seed oil, celery seed oil, cumin seed oil, seed oil nutmeg, basil leaf oil, bay leaf oil, cinnamon leaf oil, common sage leaf oil, eucalyptus leaf oil, lemon leaf oil, melaleuca leaf oil, oregano oil, patchouli oil, peppermint oil, pine needle oil, rosemary leaf oil, spearmint oil, tea tree leaf oil, thyme leaf oil, Canada tea leaf oil, flower oil, chamomile oil, roman sage oil, clove oil, ac geranium flower eite, hyssop flower oil, jasmine flower oil, lavender flower oil, mauka flower oil, marjoram flower oil, orange flower oil, rose flower oil, oil ylang-ylang flower, bark oil, cassia bark oil, cinnamon bark oil, sassafras bark oil, wood oil, camphor wood oil, cedar wood oil, rosewood oil, sandalwood oil, gingerwood oil, resin oil, recin oil, myrrh oil, skin oil, Bergamo skin oil, grapefruit skin oil, lemon skin oil, lime skin oil, orange peel oil, tangerine skin oil, root oil, valerian oil, oleic acid, linoleic acid, oleyl alcohol, isostearyl alcohol, ethyl oleate, long-chain monoglycerides, long-chain diglycerides, long-chain triglycerides, medium-chain monoglycerides, medium-chain diglycerides, medium-chain triglycerides, synthetic or semi-synthetic derivatives and combinations thereof; and
c) a surfactant, wherein the surfactant is an oil-soluble surfactant.
